SEQUENCE LISTING (217 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 3 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 4 - DNA, 594 bp, Mus musculus
atggagggtgaaggagtgcagcctctcgacgagaacctcgaaaatggcagtaggccccggttcaaatggaagaagacactgcggctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 5 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - DNA, 780 bp, Mus musculus
atgatagagacttattcccagcccagccctagaagtgttgcaaccgggctcccagcctctatgaagattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 7 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 8 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 9 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 10 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 11 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 12 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 13 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 14 - protein, 283 aa, Homo sapiens [sequence omitted]

SEQ ID NO: 15 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 16 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 17 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 18 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 20 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 21 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 22 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 23 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 24 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 25 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 26 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 27 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 28 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 29 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 30 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 31 - protein, 1154 aa, Homo sapiens [sequence omitted]

SEQ ID NO: 32 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 33 - protein, 244 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSPPIDL

SEQ ID NO: 34 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 35 - protein, 537 aa, Homo sapiens
MGCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 36 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 37 - protein, 262 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 38 - protein, 893 aa, Homo sapiens
MATEEFIIRIPPYHYIHVLDQNSNVSRVEVGPKTYIRQDNERVLFAPMRMVTVPPRHYCTVANPVSRDAQGLVLFDVTGQVRLRHADLEIRLAQDPFPLYPGEVLEKDITPLQVVLPNTALHLKALLDFEDKDGDKVVAGDEWLFEGPGTYIPRKEVEVVEIIQATIIRQNQALRLRARKECWDRDGKERVTGEEWLVTTVGAYLPAVFEEVLDLVDAVILTEKTALHLRARRNFRDFRGVSRRTGEEWLVTVQDTEAHVPDVHEEVLGVVPITTLGPHNYCVILDPVGPDGKNQLGQKRVVKGEKSFFLQPGEQLEQGIQDVYVLSEQQGLLLRALQPLEEGEDEEKVSHQAGDHWLIRGPLEYVPSAKVEVVEERQAIPLDENEGIYVQDVKTGKVRAVIGSTYMLTQDEVLWEKELPPGVEELLNKGQDPLADRGEKDTAKSLQPLAPRNKTRVVSYRVPHNAAVQVYDYREKRARVVFGPELVSLGPEEQFTVLSLSAGRPKRPHARRALCLLLGPDFFTDVITIETADHARLQLQLAYNWHFEVNDRKDPQETAKLFSVPDFVGDACKAIASRVRGAVASVTFDDFHKNSARIIRTAVFGFETSEAKGPDGMALPRPRDQAVFPQNGLVVSSVDVQSVEPVDQRTRDALQRSVQLAIEITTNSQEAAAKHEAQRLEQEARGRLERQKILDQSEAEKARKELLELEALSMAVESTGTAKAEAESRAEAARIEGEGSVLQAKLKAQALAIETEAELQRVQKVRELELVYARAQLELEVSKAQQLAEVEVKKFKQMTEAIGPSTIRDLAVAGPEMQVKLLQSLGLKSTLITDGSTPINLFNTAFGLLGMGPEGQPLGRRVASGPSPGEGISPQSAQAPQAPGDNHVVPVLR

SEQ ID NO: 39 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 40 - protein, 141 aa, Homo sapiens
MSNMRCVDCGTCLGHTRRHPTLFWGKTLPGLTPVAAPAPQPAQCPPGSEEDAPATQPGPQLAGPDPPWAPVFCRRLARAGHIELCNAVGCVLWSCPRSPGRGHAVGQGLEGGGGCPWATPPSLGGADFCNTKYSLWQKKCL

SEQ ID NO: 41 - protein, 2471 aa, Homo sapiens
MPALRPALLWALLALWLCCATPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCLQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDHMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 42 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 43 - protein, 1999 aa, Homo sapiens
MQPPSLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPGCEVNPDNCVSHQCQNGGTCQDGLDTYTCLCPETWTGWDCSEDVDECEAQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 44 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 45 - protein, 607 aa, Homo sapiens
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 46 - protein, 533 aa, Homo sapiens
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 47 - protein, 281 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSRLRAPSSSLGAFFRGDLPPPLQAGAA

SEQ ID NO: 48 - protein, 121 aa, Homo sapiens [sequence omitted]

SEQ ID NO: 49 - protein, 124 aa, Homo sapiens [sequence omitted]

SEQ ID NO: 50 - protein, 186 aa, Homo sapiens
MSGISGCPFFLWGLLALLGLALVISLIFNISHYVEKQRQDKMYSYSSDHTRVDEYYIEDTPIYGNLDDMISEPMDENCYEQMKARPEKSVNKMQEATPSAQATNETQMCYASLDHSVKGKRRKPRKQNTHFSDKDGDEQLHAIDASVSKTTLVDSFSPESQAVEENIHDDPIRLFGLIRAKREPIN

SEQ ID NO: 51 - protein, 493 aa, Homo sapiens
MRLGPRWKGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 52 - protein, 1203 aa, Homo sapiens [sequence omitted]

SEQ ID NO: 53 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPQQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 54 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTGQPTNQSMDDTREDIYVNYPTFSRRPKTRV

SEQ ID NO: 55 - protein, 393 aa, Homo sapiens
MWWRVLSLLAWFPLQEASLTNHTETITVEEGQTLTLKCVTSLRKNSSLQWLTPSGFTIFLNEYPVLKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSGILLLTLVSFLIFILFIIVQLFIMKLRKTHVIWKKENEVSEHTLESYRSRSNNEETSSEEKNGQSSHPMRCMNYITKLYSEGKTKRKENVQHSKLEEKHIQVPESIV

SEQ ID NO: 56 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 57 - protein, 405 aa, Homo sapiens [sequence omitted]

SEQ ID NO: 58 - protein, 280 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQGRIFPEGKMWKLTFSPPGTEAIYPRFS

SEQ ID NO: 59 - protein, 296 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 60 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 61 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - protein, 206 aa, Mus musculus
RTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 63 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 64 - DNA, 444 bp, Mus musculus
agctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 65 - protein, 215 aa, Homo sapiens
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 66 - DNA, 642 bp, Mus musculus
cataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 67 - protein, 164 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQI

SEQ ID NO: 68 - protein, 186 aa, Homo sapiens [sequence omitted]

SEQ ID NO: 69 - protein, 172 aa, Homo sapiens
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 70 - protein, 361 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 71 - protein, 173 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 72 - protein, 158 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 73 - protein, 182 aa, Homo sapiens
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 74 - protein, 164 aa, Homo sapiens [sequence omitted]

SEQ ID NO: 75 - protein, 175 aa, Homo sapiens
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 76 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 77 - protein, 84 aa, Homo sapiens [sequence omitted]

SEQ ID NO: 78 - protein, 9 aa, Homo sapiens
QSFGLLDPK

SEQ ID NO: 79 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 80 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 81 - protein, 111 aa, Homo sapiens
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 82 - protein, 131 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 83 - protein, 217 aa, Homo sapiens
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 84 - protein, 120 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 85 - protein, 137 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEP

SEQ ID NO: 86 - protein, 29 aa, Homo sapiens
TTPGERSSLPAFYPGTSGSCSGCGSLSLP

SEQ ID NO: 87 - protein, 15 aa, Homo sapiens
QAQAQSDCSCSTVSP

SEQ ID NO: 88 - protein, 28 aa, Homo sapiens
MEYASDASLDPEAPWPPAPRARACRVLP

SEQ ID NO: 89 - protein, 81 aa, Mus musculus
DQHTLDVEDTADARHPAGTSCPSDAALLRDTGLLADAALLSDTVRPTNAALPTDAAYPAVNVRDREAAWPPALNFCSRHPK

SEQ ID NO: 90 - protein, 23 aa, Homo sapiens
MERVQPLEENVGNAARPRFERNK

SEQ ID NO: 91 - DNA, 81 bp, Mus musculus
gagggtgaaggagtgcagcctctcgacgagaacctcgaaaatggcagtaggccccggttcaaatggaagaagacactgcgg

SEQ ID NO: 92 - protein, 22 aa, Homo sapiens
MIETYNQTSPRSAATGLPISMK

SEQ ID NO: 93 - DNA, 63 bp, Mus musculus
atagagacttattcccagcccagccctagaagtgttgcaaccgggctcccagcctctatgaag

SEQ ID NO: 94 - protein, 42 aa, Homo sapiens
KRGRKKLLYIFKQPFMRPVQTTQEEDGCSCRFPEEEEGGCEL

SEQ ID NO: 95 - protein, 42 aa, Homo sapiens
ALYLLRRDQRLPPDAHKPPGGGSFRTPIQEEQADAHSTLAKI

SEQ ID NO: 96 - protein, 48 aa, Homo sapiens
QRRKYRSNKGESPVEPAEPCHYSCPREEEGSTIPIQEDYRKPEPACSP

SEQ ID NO: 97 - protein, 189 aa, Homo sapiens
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 98 - protein, 62 aa, Homo sapiens
KKVAKKPTNKAPHPKQEPQEINFPDDLPGSNTAAPVQETLHGCQPVTQEDGKESRISVQERQ

SEQ ID NO: 99 - protein, 30 aa, Homo sapiens
HWQRTRRRTPRPGEQVPPVPSPQDLLLVEH

SEQ ID NO: 100 - protein, 37 aa, Homo sapiens
MEESVVRPSVFVVDGQTDIPFTRLGRSHRRQSCSVAR

SEQ ID NO: 101 - protein, 60 aa, Homo sapiens
CVKRRKPRGDVVKVIVSVQRKRQEAEGEATVIEALQAPPDVTTVAVEETIPSFTGRSPNH

SEQ ID NO: 102 - protein, 197 aa, Homo sapiens
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 103 - protein, 55 aa, Homo sapiens
KNRKAKAKPVTRGAGAGGRQRGQNKERPPPVPNPDYEPIRKGQRDLYSGLNQRRI

SEQ ID NO: 104 - protein, 45 aa, Homo sapiens
GHETGRLSGAADTQALLRNDQVYQPLRDRDDAQYSHLGGNWARNK

SEQ ID NO: 105 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 106 - protein, 13 aa, Homo sapiens
TWQRRQRKSRRTI

SEQ ID NO: 107 - protein, 41 aa, Homo sapiens
RSKRSRLLHSDYMNMTPRRPGPTRKHYQPYAPPRDFAAYRS

SEQ ID NO: 108 - protein, 61 aa, Homo sapiens
RKRWQNEKLGLDAGDEYEDENLYEGLNLDDCSMYEDISRGLQGTYQDVGSLNIGDVQLEKP

SEQ ID NO: 109 - protein, 49 aa, Homo sapiens
LDKDDSKAGMEEDHTYEGLDIDQTATYEDIVTLRTGEVKWSVGEHPGQE

SEQ ID NO: 110 - protein, 77 aa, Homo sapiens
QLRRRGKTNHYQTTVEKKSLTIYAQVQKPGPLQKKLDSFPAQDPCTTIYVAATEPVPESVQETNSITVYASVTLPES

SEQ ID NO: 111 - protein, 38 aa, Homo sapiens
CWLTKKKYSSSVHDPNGEYMFMRAVNTAKKSRLTDVTL

SEQ ID NO: 112 - protein, 58 aa, Homo sapiens
QLGLHIWQLRSQCMWPRETQLLLEVPPSTEDARSCQFPEEERGERSAEEKGRLGDLWV

SEQ ID NO: 113 - protein, 24 aa, Homo sapiens
LCARPRRSPAQEDGKVYINMPGRG

SEQ ID NO: 114 - protein, 52 aa, Homo sapiens
YFLGRLVPRGRGAAEAATRKQRITETESPYQELQGQRSDVYSDLNTQRPYYK

SEQ ID NO: 115 - protein, 21 aa, Homo sapiens
WALVAGLLLLLLLAAACAVFL

SEQ ID NO: 116 - protein, 21 aa, Mus musculus
LYGLVALVLLLLIAACVPIFT

SEQ ID NO: 117 - protein, 27 aa, Homo sapiens
LLLVASVIQGLGLLLCFTYICLHFSAL

SEQ ID NO: 118 - DNA, 66 bp, Mus musculus
ctggtagtgtctggaataaaaggtgcaggtatgttgctgtgcttcatctatgtttgccttcaattg

SEQ ID NO: 119 - protein, 24 aa, Homo sapiens
IFMYLLTVFLITQMIGSALFAVYL

SEQ ID NO: 120 - DNA, 72 bp, Mus musculus
attttcatgtatctgctgaccgtctttctcatcactcagatgattgggagtgtcctgttcgcggtctatctg

SEQ ID NO: 121 - protein, 27 aa, Homo sapiens
IISFFLALTSTALLFLLFFLTLRFSVV

SEQ ID NO: 122 - protein, 21 aa, Homo sapiens
VAAILGLGLVLGLLGPLAILL

SEQ ID NO: 123 - protein, 21 aa, Homo sapiens
ILVIFSGMFLVFTLAGALFLH

SEQ ID NO: 124 - protein, 27 aa, Homo sapiens
PVLDAGPVLFWVILVLVVVVGSSAFLL

SEQ ID NO: 125 - protein, 22 aa, Homo sapiens
ALVVIPIIFGILFAILLVLVFI

SEQ ID NO: 126 - protein, 21 aa, Homo sapiens
PLLLLLLLPVGLLLLAAAWCL

SEQ ID NO: 127 - protein, 21 aa, Homo sapiens
VGLGLLLLLMGAGLAVQGWFL

SEQ ID NO: 128 - protein, 21 aa, Homo sapiens
WWFLSGSLVIVIVCSTVGLII

SEQ ID NO: 129 - protein, 21 aa, Homo sapiens
MFWVQVLLAGLVVPLLLGATL

SEQ ID NO: 130 - protein, 26 aa, Homo sapiens
VMSVATIVIVDICITGGLLLLVYYWS

SEQ ID NO: 131 - protein, 21 aa, Homo sapiens
GIIVTDVIATLLLALGVFCFA

SEQ ID NO: 132 - protein, 21 aa, Homo sapiens
LCYLLDGILFIYGVILTALFL

SEQ ID NO: 133 - protein, 19 aa, Homo sapiens
VAVAGCVFLLISVLLLSGL

SEQ ID NO: 134 - protein, 27 aa, Homo sapiens
FWVLVVVGGVLACYSLLVTVAFIIFWV

SEQ ID NO: 135 - protein, 22 aa, Homo sapiens
IITAEGIILLFCAVVPGTLLLF

SEQ ID NO: 136 - protein, 21 aa, Homo sapiens
GIIMIQTLLIILFIIVPIFLL

SEQ ID NO: 137 - protein, 21 aa, Homo sapiens
WAVYAGLLGGVIMILIMVVIL

SEQ ID NO: 138 - protein, 21 aa, Homo sapiens
FWLPIGCAAFVVVCILGCILI

SEQ ID NO: 139 - protein, 21 aa, Homo sapiens
LGWLTVVLLAVAACVLLLTSA

SEQ ID NO: 140 - protein, 21 aa, Homo sapiens
LLAGLVAADAVASLLIVGAVF

SEQ ID NO: 141 - protein, 21 aa, Homo sapiens
GVLAGIVMGDLVLTVLIALAV

SEQ ID NO: 142 - protein, 148 aa, Homo sapiens
NILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYM

SEQ ID NO: 143 - protein, 227 aa, Artificial Sequence
  truncated 41BBL
MWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 144 - protein, 228 aa, Artificial Sequence
  truncated murine 41BBL
MLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 145 - protein, 161 aa, Artificial Sequence
  truncated OX40L
MLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 146 - DNA, 513 bp, Artificial Sequence
  truncated murine  OX40L
atgctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 147 - protein, 240 aa, Artificial Sequence
  truncated CD40L
MIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 148 - DNA, 717 bp, Artificial Sequence
  truncated murine CD40L
atgattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 149 - protein, 205 aa, Artificial Sequence
  truncated human FLT3L
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCL

SEQ ID NO: 150 - protein, 204 aa, Artificial Sequence
  truncated human LIGHT
MVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 151 - protein, 269 aa, Artificial Sequence
  41BBL-OX40 DCR
MALYLLRRDQRLPPDAHKPPGGGSFRTPIQEEQADAHSTLAKIWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 152 - protein, 264 aa, Artificial Sequence
  mu41BBL-OX40 DCR
MRKAWRLPNTPKPCWGNSFRTPIQEEHTDAHFTLAKILYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 153 - protein, 275 aa, Artificial Sequence
  41BBL-CD27 DCR
MQRRKYRSNKGESPVEPAEPCHYSCPREEEGSTIPIQEDYRKPEPACSPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 154 - protein, 275 aa, Artificial Sequence
  mu41BBL-CD27 DCR
MQRRNHGPNEDRQAVPEEPCPYSCPREEEGSAIPIQEDYRKPEPAFYPLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 155 - protein, 416 aa, Artificial Sequence
  41BBL-CD30 DCR
MCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 156 - protein, 418 aa, Artificial Sequence
  mu41BBL-CD30 DCR
MYWKACRRRFQQKFHLDYLVQTFQPKMEQTDSCPTEKLTQPQRSGSVTDPSTGHKLSPVSPPPAVETCASVGATYLENLPLLDDSPAGNPFSPREPPEPRVSTEHTNNRIEKIYIMKADTVIVGSVKTEVPEGRAPAGSTESELEAELEVDHAPHYPEQETEPPLGSCTEVMFSVEEGGKEDHGPTTVSEKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 157 - protein, 289 aa, Artificial Sequence
  41BBL-CD40 DCR
MKKVAKKPTNKAPHPKQEPQEINFPDDLPGSNTAAPVQETLHGCQPVTQEDGKESRISVQERQWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 158 - protein, 302 aa, Artificial Sequence
  mu41BBL-CD40 DCR
MKKVVKKPKDNEILPPAARRQDPQEMEDYPGHNTAAPVQETLHGCQPVTQEDGKESRISVQERQVTDSIALRPLVLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 159 - protein, 287 aa, Artificial Sequence
  41BBL-HVEM DCR
MCVKRRKPRGDVVKVIVSVQRKRQEAEGEATVIEALQAPPDVTTVAVEETIPSFTGRSPNHWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 160 - protein, 272 aa, Artificial Sequence
  mu41BBL-HVEM DCR
MTRRHLHTSSVAKELEPFQEQQENTIRFPVTEVGFAETEEETASNLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 161 - protein, 424 aa, Artificial Sequence
  41BBL-DR3 DCR
MTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 162 - protein, 426 aa, Artificial Sequence
  mu41BBL-DR3 DCR
MICAYCRWQPCKAVVTADTAGTETLASPQTAHLSASDSAHTLLAPPSSTGKICTTVQLVGNNWTPGLSQTQEVVCGQASQPWDQLPNRTLGTPLASPLSPAPPAGSPAAVLQPGPQLYDVMDAVPARRWKEFVRTLGLREAEIEAVEVEICRFRDQQYEMLKRWRQQQPAGLGAIYAALERMGLEGCAEDLRSRLQRGPLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 163 - protein, 269 aa, Artificial Sequence
  41BBL-41BB DCR
MKRGRKKLLYIFKQPFMRPVQTTQEEDGCSCRFPEEEEGGCELWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 164 - protein, 273 aa, Artificial Sequence
  mu41BBL-41BB DCR
MSVLKWIRKKFPHIFKQPFKKTAQEEDACSCRCPQEEEGGGGGYELLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 165 - protein, 203 aa, Artificial Sequence
  OX40L-OX40 DCR
MALYLLRRDQRLPPDAHKPPGGGSFRTPIQEEQADAHSTLAKILLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 166 - DNA, 621 bp, Artificial Sequence
  muOX40L-OX40 DCR
atgaggaaggcatggcggcttcctaatacacccaagccttgctggggtaattccttcaggactcccatacaggaggaacatacagacgcccatttcacattggcaaagatactggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 167 - protein, 209 aa, Artificial Sequence
  OX40L-CD27 DCR
MQRRKYRSNKGESPVEPAEPCHYSCPREEEGSTIPIQEDYRKPEPACSPLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 168 - DNA, 654 bp, Artificial Sequence
  muOX40L-CD27 DCR
atgcagcgccgaaatcacggacctaacgaagacagacaggcagtaccagaggagccttgcccatatagttgtccaagagaggaggaaggttccgctattcccatccaggaggattatcgcaagcctgagcctgcattctacccactggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 169 - protein, 350 aa, Artificial Sequence
  OX40L-CD30 DCR
MCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 170 - DNA, 1083 bp, Artificial Sequence
  muOX40L-CD30 DCR
atgtactggaaagcatgccgaagaaggttccagcaaaagtttcatcttgactaccttgtacaaacctttcagccaaagatggagcaaactgattcatgtccaaccgagaaattgacacaacctcagcgcagtgggtccgtaactgaccctagcacagggcacaagctctctcctgtgtctccaccacccgccgttgaaacttgtgcatctgtcggagctacctatttggaaaacctccctctgctcgatgactcacctgctggtaatccattttctcccagagagccccccgagcctagagtttccaccgaacatactaataaccggattgaaaaaatatatattatgaaagccgacaccgtgatagtaggtagtgtaaaaacagaggtccctgagggtagagctccagccggctcaaccgagtcagagctggaggctgaattggaagtagatcacgcaccacattatcccgagcaggaaactgagcctcctctcggatcttgtaccgaagtgatgttctccgtagaggaaggtgggaaggaggaccacggaccaacaacagtgtccgagaagctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 171 - protein, 223 aa, Artificial Sequence
  OX40L-CD40 DCR
MKKVAKKPTNKAPHPKQEPQEINFPDDLPGSNTAAPVQETLHGCQPVTQEDGKESRISVQERQLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 172 - DNA, 735 bp, Artificial Sequence
  muOX40L-CD40 DCR
atgaaaaaagtggttaagaaaccaaaggacaacgaaatccttcctcccgcagcacgacgacaagaccctcaagagatggaagactatcccggccacaacaccgcagctccagtccaagaaacccttcatgggtgtcaacctgtaacacaggaagacgggaaagagtcaaggatttcagtgcaagagcgacaagtgactgacagcatcgctttgaggccactggttctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 173 - protein, 221 aa, Artificial Sequence
  OX40L-HVEM DCR
MCVKRRKPRGDVVKVIVSVQRKRQEAEGEATVIEALQAPPDVTTVAVEETIPSFTGRSPNHLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 174 - DNA, 645 bp, Artificial Sequence
  muOX40L-HVEM DCR
atgactagacgccacctccacaccagctcagtagcaaaggagctggaaccatttcaagagcagcaggaaaatacaattcggtttcctgtaaccgaggttggctttgctgagactgaggaagaaactgctagtaatctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 175 - protein, 358 aa, Artificial Sequence
  OX40L-DR3 DCR
MTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 176 - DNA, 1107 bp, Artificial Sequence
  muOX40L-DR3 DCR
atgatttgcgcctactgcagatggcagccttgcaaggcagtagttactgctgataccgctggcacagaaactttggcatccccacagactgctcatctgtccgcatctgattccgctcatactctcctcgcccctcctagcagtacaggaaaaatatgtactaccgtgcaactcgtcggaaacaactggacccccgggctttctcaaactcaagaggtcgtatgtgggcaggcttcacaaccatgggatcaactgccaaataggacccttggtacaccactggcttcacctctgtctcccgcccccccagctggatctcctgccgccgtccttcagcccggtccccagctctacgacgtaatggatgctgtacctgctaggagatggaaagagttcgtgaggaccctcggtctgagggaagctgagatcgaagccgtagaagtggaaatctgccgattccgggatcaacagtatgagatgctcaagcgatggaggcaacaacagcccgccgggctgggcgctatttacgctgctcttgagcgcatgggactggagggatgcgctgaagatctgagatcaaggctccagcgggggcccctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 177 - protein, 203 aa, Artificial Sequence
  OX40L-41BB DCR
MKRGRKKLLYIFKQPFMRPVQTTQEEDGCSCRFPEEEEGGCELLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 178 - DNA, 657 bp, Artificial Sequence
  muOX40L-41BB DCR
atgtccgttctcaaatggatcagaaaaaagttcccccacatctttaaacaaccattcaagaaaaccactggggcagctcaagaggaggacgcttgctcttgtcggtgtccacaggaagaggaaggtggtggcggtggttacgagttgctggtagtgtctggaataaaaggtgcaggtatgttgctgtgcttcatctatgtttgccttcaattgagctctagccctgctaaagacccacctatccagcggctcaggggagcagtaacacgctgtgaagacggtcagttgtttattagttcttacaagaacgagtaccagacaatggaagttcaaaataattctgtcgtcatcaaatgtgacgggttgtatataatatatctgaaaggcagtttttttcaagaggtaaagatcgacctgcactttcgagaggatcataatccaatttctatacctatgctcaatgatgggaggcgcatcgtcttcaccgtagtggccagcctcgcttttaaggacaaagtttatctcactgttaatgcacccgatacactgtgcgagcacctgcaaattaacgatggagagcttatagttgtgcaacttactcctggatattgcgctcccgaagggtcttatcatagtactgtaaaccaagtacccctt

SEQ ID NO: 179 - protein, 282 aa, Artificial Sequence
  CD40L-OX40 DCR
MALYLLRRDQRLPPDAHKPPGGGSFRTPIQEEQADAHSTLAKIIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 180 - DNA, 825 bp, Artificial Sequence
  muCD40L-OX40 DCR
atgaggaaggcatggcggcttcctaatacacccaagccttgctggggtaattccttcaggactcccatacaggaggaacatacagacgcccatttcacattggcaaagataattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 181 - protein, 288 aa, Artificial Sequence
  CD40L-CD27 DCR
MQRRKYRSNKGESPVEPAEPCHYSCPREEEGSTIPIQEDYRKPEPACSPIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 182 - DNA, 858 bp, Artificial Sequence
  muCD40L-CD27 DCR
atgcagcgccgaaatcacggacctaacgaagacagacaggcagtaccagaggagccttgcccatatagttgtccaagagaggaggaaggttccgctattcccatccaggaggattatcgcaagcctgagcctgcattctacccaattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 183 - protein, 429 aa, Artificial Sequence
  CD40L-CD30 DCR
MCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 184 - DNA, 1287 bp, Artificial Sequence
  muCD40L-CD30 DCR
atgtactggaaagcatgccgaagaaggttccagcaaaagtttcatcttgactaccttgtacaaacctttcagccaaagatggagcaaactgattcatgtccaaccgagaaattgacacaacctcagcgcagtgggtccgtaactgaccctagcacagggcacaagctctctcctgtgtctccaccacccgccgttgaaacttgtgcatctgtcggagctacctatttggaaaacctccctctgctcgatgactcacctgctggtaatccattttctcccagagagccccccgagcctagagtttccaccgaacatactaataaccggattgaaaaaatatatattatgaaagccgacaccgtgatagtaggtagtgtaaaaacagaggtccctgagggtagagctccagccggctcaaccgagtcagagctggaggctgaattggaagtagatcacgcaccacattatcccgagcaggaaactgagcctcctctcggatcttgtaccgaagtgatgttctccgtagaggaaggtgggaaggaggaccacggaccaacaacagtgtccgagaagattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 185 - protein, 302 aa, Artificial Sequence
  CD40L-CD40 DCR
MKKVAKKPTNKAPHPKQEPQEINFPDDLPGSNTAAPVQETLHGCQPVTQEDGKESRISVQERQIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 186 - DNA, 939 bp, Artificial Sequence
  muCD40L-CD40 DCR
atgaaaaaagtggttaagaaaccaaaggacaacgaaatccttcctcccgcagcacgacgacaagaccctcaagagatggaagactatcccggccacaacaccgcagctccagtccaagaaacccttcatgggtgtcaacctgtaacacaggaagacgggaaagagtcaaggatttcagtgcaagagcgacaagtgactgacagcatcgctttgaggccactggttattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 187 - protein, 300 aa, Artificial Sequence
  CD40L-HVEM DCR
MCVKRRKPRGDVVKVIVSVQRKRQEAEGEATVIEALQAPPDVTTVAVEETIPSFTGRSPNHIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 188 - DNA, 849 bp, Artificial Sequence
  muCD40L-HVEM DCR
atgactagacgccacctccacaccagctcagtagcaaaggagctggaaccatttcaagagcagcaggaaaatacaattcggtttcctgtaaccgaggttggctttgctgagactgaggaagaaactgctagtaatattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 189 - protein, 437 aa, Artificial Sequence
  CD40L-DR3 DCR
MTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 190 - DNA, 1311 bp, Artificial Sequence
  muCD40L-DR3 DCR
atgatttgcgcctactgcagatggcagccttgcaaggcagtagttactgctgataccgctggcacagaaactttggcatccccacagactgctcatctgtccgcatctgattccgctcatactctcctcgcccctcctagcagtacaggaaaaatatgtactaccgtgcaactcgtcggaaacaactggacccccgggctttctcaaactcaagaggtcgtatgtgggcaggcttcacaaccatgggatcaactgccaaataggacccttggtacaccactggcttcacctctgtctcccgcccccccagctggatctcctgccgccgtccttcagcccggtccccagctctacgacgtaatggatgctgtacctgctaggagatggaaagagttcgtgaggaccctcggtctgagggaagctgagatcgaagccgtagaagtggaaatctgccgattccgggatcaacagtatgagatgctcaagcgatggaggcaacaacagcccgccgggctgggcgctatttacgctgctcttgagcgcatgggactggagggatgcgctgaagatctgagatcaaggctccagcgggggcccattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 191 - protein, 282 aa, Artificial Sequence
  CD40L-41BB DCR
MKRGRKKLLYIFKQPFMRPVQTTQEEDGCSCRFPEEEEGGCELIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 192 - DNA, 861 bp, Artificial Sequence
  muCD40L-41BB DCR
atgtccgttctcaaatggatcagaaaaaagttcccccacatctttaaacaaccattcaagaaaaccactggggcagctcaagaggaggacgcttgctcttgtcggtgtccacaggaagaggaaggtggtggcggtggttacgagttgattttcatgtatctgctgaccgtctttctcatcactcagatgattgggagtgtcctgttcgcggtctatctgcataggcgcctggacaaggtcgaagaggaggtcaacctgcacgaggactttgtctttatcaagaagttgaagcggtgcaacaagggcgaaggatctctgagcctcctgaactgtgaagaaatgcgccgacagtttgaggatctggtaaaagacataacgcttaacaaggaagaaaagaaggagaattctttcgagatgcaacgcggcgatgaggatccacagatcgccgcgcacgtcgtctcagaggccaattcaaacgcagcctcagtacttcaatgggcgaaaaaggggtattacaccatgaagtctaatcttgttatgctagagaatgggaagcagctgacagtgaagcgcgaggggctgtactatgtatatacacaagttaccttctgcagcaatagggagccaagtagccagcgcccctttatcgtggggctgtggctcaaacccagcagcggctccgaacgtattctgcttaaggctgccaacactcacagttcaagccagctgtgcgagcagcagtccgtacatcttggcggagtctttgagctccaggctggcgcctcagtgttcgtgaacgttaccgaagcttctcaggttattcaccgggtgggcttttcttcctttggactgctgaaactc

SEQ ID NO: 193 - protein, 247 aa, Artificial Sequence
  huFLT3L-OX40 DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLALYLLRRDQRLPPDAHKPPGGGSFRTPIQEEQADAHSTLAKI

SEQ ID NO: 194 - protein, 253 aa, Artificial Sequence
  huFLT3L-CD27 DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLQRRKYRSNKGESPVEPAEPCHYSCPREEEGSTIPIQEDYRKPEPACSP

SEQ ID NO: 195 - protein, 394 aa, Artificial Sequence
  huFLT3L-CD30 DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 196 - protein, 267 aa, Artificial Sequence
  huFLT3L-CD40 DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLKKVAKKPTNKAPHPKQEPQEINFPDDLPGSNTAAPVQETLHGCQPVTQEDGKESRISVQERQ

SEQ ID NO: 197 - protein, 265 aa, Artificial Sequence
  huFLT3L-HVEM DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLCVKRRKPRGDVVKVIVSVQRKRQEAEGEATVIEALQAPPDVTTVAVEETIPSFTGRSPNH

SEQ ID NO: 198 - protein, 402 aa, Artificial Sequence
  huFLT3L-DR3 DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 199 - protein, 247 aa, Artificial Sequence
  huFLT3tm-41BB DCR
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLKRGRKKLLYIFKQPFMRPVQTTQEEDGCSCRFPEEEEGGCEL

SEQ ID NO: 200 - protein, 246 aa, Artificial Sequence
  huLIGHT-OX40 DCR
MALYLLRRDQRLPPDAHKPPGGGSFRTPIQEEQADAHSTLAKIVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 201 - protein, 252 aa, Artificial Sequence
  huLIGHT-CD27 DCR
MQRRKYRSNKGESPVEPAEPCHYSCPREEEGSTIPIQEDYRKPEPACSPVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 202 - protein, 393 aa, Artificial Sequence
  huLIGHT-CD30 DCR
MCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGKVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 203 - protein, 266 aa, Artificial Sequence
  huLIGHT-CD40 DCR
MKKVAKKPTNKAPHPKQEPQEINFPDDLPGSNTAAPVQETLHGCQPVTQEDGKESRISVQERQVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 204 - protein, 264 aa, Artificial Sequence
  huLIGHT-HVEM DCR
MCVKRRKPRGDVVKVIVSVQRKRQEAEGEATVIEALQAPPDVTTVAVEETIPSFTGRSPNHVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 205 - protein, 401 aa, Artificial Sequence
  huLIGHT-DR3 DCR
MTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 206 - protein, 246 aa, Artificial Sequence
  huLIGHT-41BB DCR
MKRGRKKLLYIFKQPFMRPVQTTQEEDGCSCRFPEEEEGGCELVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 207 - DNA, 24 bp, Artificial Sequence
  Furin cleavage + SGS linker for the 3 end of the human costimulatory constructs
cgtgcaaaacgttcaggctcaggt

SEQ ID NO: 208 - protein, 107 aa, Artificial Sequence
  FKBP12 domain
MGVQVETISPGDGRTFPKRGQTCWHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 209 - protein, 100 aa, Artificial Sequence
  Wild-type FRB segment of mTOR
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 210 - protein, 100 aa, Artificial Sequence
  FRB (with T to L substitution at 2098 which allows binding to AP21967)
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLES

SEQ ID NO: 211 - protein, 100 aa, Artificial Sequence
  FRB segment of mTOR with substitutions
MASRILWHEMWHEGLEEASRLYFGERNVKGMFEVLEPLHAMMERGPQTLKETSFNQAYGRDLMEAQEWCRKYMKSGNVKDLHQAFDLYYHVFRRISKLES

SEQ ID NO: 212 - protein, 100 aa, Artificial Sequence
  FRB segment of mTOR with substitutions
MASRILWHEMWHEGLEEASRLYFGERNVKGMFEVLEPLHAMMERGPQTLKETSFNQAYGRDLMEAQEWCRKYMKSGNVPDLTQAWDLYYHVFRRISKLES

SEQ ID NO: 213 - protein, 234 aa, Artificial Sequence
  EcR
MPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 214 - protein, 336 aa, Artificial Sequence
  RXR
RPECVVPETQCAMKRKEKKAQKEKDKLPVSITTVDDHMPPIMQCEPPPPEAARIHEVVPRFLSDKLLETNRQKNIPQLTANQQFLIARLIWYQDGYEQPSDEDLKRITQTVVQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 215 - protein, 10 aa, Artificial Sequence
  GlySer junction amino acids
GGGGSGGGGS

SEQ ID NO: 216 - protein, 9 aa, Artificial Sequence
  GlySer junction amino acids
GGSGGSGGS

SEQ ID NO: 217 - protein, 5 aa, Artificial Sequence
  GlySer junction amino acids
GGGGS

(19) United States
(12) Patent Application Publication
(10) Pub. No.: US 2024/0076351 A1
(43) Pub. Date: Mar. 7, 2024

(54) HYBRID AND TRUNCATED IMMUNE CELL PROTEINS

(71) Applicant: Seattle Children's Hospital d/b/a Seattle Children's Research Institute, Seattle, WA (US)

(72) Inventor: Shannon Oda, Seattle, WA (US)

(73) Assignee: Seattle Children's Hospital d/b/a Seattle Children's Research Institute, Seattle, WA (US)

(21) Appl. No.: 18/261,656

(22) PCT Filed: Jan. 14, 2022
(86) PCT No.: PCT/US22/12609

§ 371 Date: Jul. 14, 2023

Related U.S. Application Data
(60) Provisional application No. 63/138,234, filed on Jan. 15, 2021.

(51) Int. Cl. C07K 14/705

(52) U.S. Cl. CPC C07K 14/70575; C07K 14/70578; A61K 38/00; C07K 2319/03

(57) ABSTRACT

In Hybrid and truncated immune cell proteins are described. Hybrid proteins are stimulatory and include an extracellular domain of one stimulatory immune cell protein, an intracellular domain of a different stimulatory immune cell protein, and a transmembrane domain linking the extracellular domain to the intracellular domain. Truncated proteins include an immune cell receptor ligand and a transmembrane domain but lack a functional intracellular domain. The hybrid and truncated proteins can be used to modulate and/or diversify immune cell activation in the fight against cancers and infectious diseases, among other uses.

### CROSS-REFERENCE TO RELATED APPLICATION

This application is a U.S. National Phase based on International Patent Application No. PCT/US2022/012609, filed Jan. 14, 2022, which claims priority to U.S. Provisional Patent Application No. 63/138,234 filed on Jan. 15, 2021, each of which is incorporated herein by reference in its entirety as if fully set forth herein.

### REFERENCE TO SEQUENCE LISTING

The Sequence Listing associated with this application is provided in text format in lieu of a paper copy and is hereby incorporated by reference into the specification. The name of the text file containing the Sequence Listing is 2VWV5188_ST25.bd. The text file is 427 KB, was created on Jun. 15, 2023, and is being submitted electronically via Patent Center.

### FIELD OF THE DISCLOSURE

The current disclosure provides hybrid and truncated immune cell proteins. Hybrid immune cell proteins are stimulatory and include an extracellular domain of one stimulatory immune cell protein, an intracellular domain of a different stimulatory immune cell protein, and a transmembrane domain linking the extracellular domain to the intracellular domain. Truncated immune cell proteins include immune cell ligands that lack a functional intracellular domain. The hybrid and truncated proteins can be used to modulate and/or diversify immune cell activation in the fight against cancers and infectious diseases, among other uses.

### BACKGROUND OF THE DISCLOSURE

The immune system uses two general mechanisms to protect the body against cancerous cells or environmental pathogens such as viruses, bacteria, and fungi. One is the non-specific (or innate) inflammatory response. The other is the specific (acquired or adaptive) immune response. In contrast to the innate response which is fundamentally the same for each injury, acquired responses are custom tailored to particular cancers or pathogens.

The immune system can recognize and respond to differences between healthy/self and unhealthy/non-self-antigen, including antigens on cancerous cells. Acquired immunity has specific “memory” for these recognized antigens, and repeated exposure to the same antigen increases the response increases the level of induced protection against these previously-encountered cancers or pathogens.

Acquired immunity is mediated by the specialized immune cells, B and T lymphocytes. B lymphocytes produce and mediate their functions through the actions of antibodies. B lymphocyte dependent immune responses are referred to as “humoral immunity” because antibodies are detected in body fluids. T lymphocyte dependent immune responses are referred to as “cell mediated immunity” because effector activities are mediated directly by the local actions of effector T lymphocytes. The local actions of effector T lymphocytes are amplified through synergistic interactions between T lymphocytes and secondary effector cells, such as activated macrophages.

In order for immune cells to respond to foreign antigens, two signals must be provided by antigen-presenting cells (APCs) to resting T lymphocytes. The first signal, which confers specificity to the immune response, is transduced via the T cell receptor (TCR) when the TCR engages a specific peptide presented in the context of the major histocompatibility complex (MHC) (Rossy et al., Frontiers in Immunol. 3: 1-12, 2012). The second signal, or co-stimulatory signal, is an antigen-independent co-signal that directs T cell function and T cell fate (Lenschow et al. (1996) Annu. Rev. Immunol. 14:233) (Chen and Flies Nat Rev Imm 2013). A co-stimulatory signal can be provided by temporary binding to one or more distinct cell surface polypeptides expressed by APCs (Jenkins, M. K. et al. (1988) J. Immunol. 140:3324-3330).

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

Clinical trials with CAR-expressing T cells (CAR-T) have shown positive responses and cancer reduction in some patients. However, in others they have failed to provide prolonged anti-cancer activity. One reason for a lack of prolonged success may be that the T cells do not receive strong or diverse enough activation signals when bound to a targeted cell.

### SUMMARY OF THE DISCLOSURE

The current disclosure provides hybrid and truncated proteins that modulate and diversify immune cell activation. The hybrid proteins are stimulatory and include an extracellular domain of one stimulatory immune cell protein, an intracellular domain of a different stimulatory immune cell protein, and a transmembrane domain linking the extracellular domain to the intracellular domain. Truncated proteins include immune cell ligands with an extracellular domain and a transmembrane domain that lack a functional intracellular domain. When expressed by immune cells, the disclosed hybrid proteins allow two or more stimulatory signals to be activated in the presence of a particular ligand, as explained more fully below. When expressed by immune cells, the disclosed truncated proteins can act as decoys further modulating the strength of the immune system response.

Immune cell proteins that can be used to create hybrid stimulatory proteins described herein include 4-1BB (CD137), 4-1BBL (CD137L), OX40 (CD134), OX40L (CD134L), CD40, CD40L, CD30, CD30L, CD27, CD27L, DR3, DR3L, HVEM, LIGHT, SLAMF1 (CD150 or SLAM), SLAMF1L, ICOS (CD278), ICOSL, GITR (CD357), GITRL, BLAME (SLAMF8), CD2, CD25, CD122, CD132, CD28, CD80, CD86, CRTAM (CD355), CD79A, CD79AL, CD79B, CD79BL, CD84 (SLAMF5), CD226, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, Ly9 (SLAMF3), NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, SLAMF7 (CRACC or CD319)), Slp76, Slp76L, pTα, pTαL, TCRα, TCRβ, HAVCR1 (TIM1), TIM1L, TRIM, TRIML, Zap70, Zap70L, PTCH2, PTCH2L, CD3ε; CD3δ; and CD3ζ.

In some examples, hybrid proteins are formed from co-stimulatory proteins such as 4-1BB, 4-1BBL, OX40, OX40L, CD40, CD40L, CD30, CD30L, CD27, CD27L, DR3, DR3L, FLT3, FLT3L, HVEM, and LIGHT. In additional examples, hybrid proteins are formed from the extracellular domain of a co-stimulatory ligand protein and the intracellular domain of a co-stimulatory protein.

In one example, an immune cell naturally expresses 4-1BB and is genetically engineered to express a hybrid co-stimulatory protein including the extracellular domain of 4-1BBL and the intracellular signaling domain of OX40. In this example, in the presence of 4-1BB, the cell would receive 4-1BB and OX40 activation signals.

In another example, an immune cell naturally expresses 4-1BB and is genetically engineered to express two hybrid co-stimulatory proteins disclosed herein, for example, a hybrid co-stimulatory protein including the extracellular domain of 4-1BBL and the intracellular signaling domain of OX40 and a hybrid co-stimulatory protein including the extracellular domain of 4-1BBL and the intracellular signaling domain of CD40. In this example, in the presence of 4-1BB, the cell would receive 4-1BB, OX40, and CD40 activation signals.

Numerous hybrid stimulatory proteins can be generated in various combinations and expressed in cells to tailor activation signals triggered in the presence of a particular ligand.

Immune cell proteins that can be used to create truncated proteins described herein include 4-1BBL (CD137L), OX40L (CD134L), CD40L, CD30L, CD27L, DR3L, SLAMF1L, ICOSL, GITRL, CD80, CD86, CD79AL, CD79BL, CARD11 L, DAP10L, DAP12L, FcRαL, FcR8L, FcRγL, FLT3L, FynL, LckL, LATL, LRPL, NKG2DL, NOTCH1L, NOTCH2L, NOTCH3L, NOTCH4L, ROR2L, RykL, Slp76L, pTαL, TIM1L, TRIML, Zap70L, and PTCH2L. Truncated proteins disclosed herein can be truncated forms of natural proteins or can also be made hybrid by having the extracellular domain of an immune cell receptor ligand and the transmembrane domain of a different protein.

Immune system activation modulation and diversification based on expression of the hybrid and/or truncated proteins disclosed herein can be further refined using, for example, chemically-induced multimerization systems (CIMS) and/or inducible expression.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A, 1B. Schematics. (1A) Schematics of exemplary hybrid stimulatory proteins (also referred to as dual costimulatory receptors (DCR) herein) including an extracellular domain of one stimulatory protein, a transmembrane domain, and at least one intracellular domain of a different stimulatory protein. (1B) Schematics of exemplary truncated proteins including an extracellular domain of an immune receptor ligand and a transmembrane domain, wherein the truncated protein lacks a functional intracellular domain. A hybrid truncated protein is depicted wherein the truncated protein includes a transmembrane domain from a different protein. The transmembrane domain can be derived from the same protein as the extracellular component or can be derived from a different protein.

FIG. 2. Depiction of approach used to fuse Type I and Type II protein domains. In this depiction, the intracellular signaling domain of a Type I co-stimulatory protein is inverted so that the intracellular tail terminus becomes membrane proximal.

FIGS. 3A-3C. Expression data for exemplary hybrid co-stimulatory proteins including (3A) 4-1BBL-CD30, (3B) 4-1BBL-CD40, and (3C) 4-1BBL-OX40.

FIGS. 4A-4D. Jurkat transduction screen data for (3A) 4-1BBL-CD27, (3B) 4-1BBL-DR3, and (3C) tr4-1BBL, and (3D) 4-1BBL-4-1BB.

FIG. 5. OX40L hybrid co-stimulatory protein expression results in increased activation in T cells. Jurkat T cells were engineered to express a T-cell receptor (TCR) only or TCR/DCR by lentiviral transduction. T cells were then stimulated with anti-CD3 (OKT3, 0.3 ug/mL) in co-culture with T2 tumor cells. On day 3 post-stimulation, T cells were stained with anti-OX40L and anti-CD69 antibodies and analyzed by flow cytometry. Engineered T cells exhibited variable OX40L DCR expression, as detected by specific anti-OX40L antibody; listed from highest to lowest expression: OX40L-CD30, OX40L-HVEM, OX40L-CD40, and OX40L-4-1BB. Despite variable expression, OX40L DCR expression resulted in enhanced activation, as determined by CD69 expression (activation marker), relative to control T cells (TCR only).

FIG. 6. Jurkat T cells were engineered to express TCR only or TCR/DCR by lentiviral transduction. T cells were then stimulated with anti-CD3 (OKT3, 0.3 ug/mL) in co-culture with T2 tumor cells. On day 3 post-stimulation, T cells were stained with anti-4-1BBL and anti-4-1BB antibodies and analyzed by flow cytometry. Engineered T cells exhibited variable 4-1BBL DCR expression. 4-1BBL DCR expression resulted in enhanced activation, as determined by increased 4-1BB expression (activation marker), relative to control T cells (TCR only).

FIG. 7. 4-1BBL DCR expression in Jurkat T cells. Jurkat T cells were transduced with a lentiviral vector including a TCR and the indicated transgene. Cells were stained with specific tetramer to identify transduced T cells and fluorescence-activated cell sorting (FACS)-sorted. T cells were subsequently stained with specific antibody to 4-1BBL and analyzed by flow cytometry. T cell only, untransduced; Vector only, TCR only; FL, full-length; Tr, truncated.

FIG. 8. OX40L DCR expression in Jurkat T cells. Jurkat T cells were transduced with a lentiviral vector including a TCR and the indicated transgene. Cells were stained with specific tetramer to identify transduced T cells and FACS-sorted. T cells were subsequently stained with specific antibody to OX40L and analyzed by flow cytometry.

FIG. 9. CD40L DCR expression in Jurkat T cells. Jurkat T cells were transduced with a lentiviral vector including a TCR and the indicated transgene. Cells were stained with specific tetramer to identify transduced T cells and FACS-sorted. T cells were subsequently stained with specific antibody to CD40L and analyzed by flow cytometry.

FIG. 10. FLT3L DCR expression in Jurkat T cells. Jurkat T cells were transduced with a lentiviral vector including a TCR and the indicated transgene. Cells were stained with specific tetramer to identify transduced T cells and FACS-sorted. T cells were subsequently stained with specific antibody to FLT3L and analyzed by flow cytometry.

FIG. 11. Engineered OX40L DCRs exhibit surface expression in engineered primary human CD8 T cells. Six days post-transduction, engineered primary human CD8 T cells were stained with specific antibodies and analyzed by flow cytometry. Several DCRs exhibit surface expression as detectable by specific antibody detection and flow cytometry.

FIG. 12. Engineered 4-1BBL DCRs exhibit surface expression in engineered primary human CD8 T cells. Six days post-transduction, engineered primary human CD8 T cells were stained with specific antibodies and analyzed by flow cytometry. Several DCRs exhibit surface expression as detectable by specific antibody detection and flow cytometry.

FIG. 13. Activation in 4-1BBL DCR-engineered Jurkat T cells. Jurkat T cells were transduced with a lentiviral vector including a TCR and the indicated 4-1BBL DCR. Cells were stained with specific tetramer to identify transduced T cells and FACS-sorted. T cells were subsequently stimulated with 0.3 ug/mL anti-CD3 (OKT3) for 16 hours, then stained with specific antibodies to the activation marker, CD69, and analyzed by flow cytometry. 4-1BBL DCRs enhance T cell activation.

FIG. 14. Activation in OX40L DCR-engineered Jurkat T cells. Jurkat T cells were transduced with a lentiviral vector including a TCR and the indicated OX40L DCR. Cells were stained with specific tetramer to identify transduced T cells and FACS-sorted. T cells were subsequently stimulated with 0.3 ug/mL anti-CD3 (OKT3) for 16 hours, then stained with specific antibodies to the activation markers, CD69 and 4-1BB, and analyzed by flow cytometry. OX40L DCRs enhance T cell activation.

FIG. 15. Activation in CD40L DCR-engineered Jurkat T cells. Jurkat T cells were transduced with a lentiviral vector including a TCR and the indicated CD40L DCR. Cells were stained with specific tetramer to identify transduced T cells and FACS-sorted. T cells were subsequently stimulated with 0.3 ug/mL anti-CD3 (OKT3) for 16 hours, then stained with specific antibodies to the activation marker, CD69, and analyzed by flow cytometry. CD40L DCRs enhance T cell activation.

FIG. 16. Activation in 4-1BBL DCR-engineered Jurkat T cells. Jurkat T cells were transduced with a lentiviral vector including a TCR and the indicated 4-1BBL DCR. Cells were stained with specific tetramer to identify transduced T cells and FACS-sorted. T cells were subsequently cultured at a 1:1 ratio with 1 ug/mL of mesothelin peptide (peptide_{MSLN})-pulsed T2 cells for 16 hours, then stained with specific antibodies to the activation marker, CD69, and analyzed by flow cytometry. 4-1BBL DCRs enhance T cell activation.

FIG. 17. Activation in OX40L DCR-engineered Jurkat T cells. Jurkat T cells were transduced with a lentiviral vector including a TCR and the indicated OX40L DCR. Cells were stained with specific tetramer to identify transduced T cells and FACS-sorted. T cells were subsequently cultured at a 1:1 ratio with 1 ug/mL peptide_{MSLN}-pulsed T2 cells for 16 hours, then stained with specific antibodies to the activation markers, CD69 and 4-1BB, and analyzed by flow cytometry. OX40L DCRs enhance T cell activation.

FIG. 18. Activation in CD40L DCR-engineered Jurkat T cells. Jurkat T cells were transduced with a lentiviral vector including a TCR and the indicated CD40L DCR. Cells were stained with specific tetramer to identify transduced T cells and FACS-sorted. T cells were subsequently cultured at a 1:1 ratio with 1 ug/mL peptide_{MSLN}-pulsed T2 cells for 16 hours, then stained with specific antibodies to the activation marker, CD69, and analyzed by flow cytometry. CD40L DCRs enhance T cell activation.

FIG. 19. OX40L DCRs enhance cytotoxic CD8+ T cell function. Lysis of Panc1 tumor cells by OX40L DCR-engineered primary human T cells. Engineered primary human CD8 T cells were cultured a 1:1 ratio with 1 ug/mL peptidems LN -pulsed NIR⁺ Panc1 cells. Tumor lysis was determined by loss of near infrared (NIR) signal quantification by IncuCyte (Sartorius) instrument and analysis, with images taken hourly. Several DCRs enhance lysis of Panc1 tumor cells, relative to control (FL). E:T, 1:1.

FIG. 20. Lysis of Panc1 tumor cells by 4-1BBL DCR-engineered primary human T cells. Engineered primary human CD8 T cells were cultured a 1:1 ratio with 1 ug/mL peptide_{MSLN}-pulsed NIR⁺ Panc1 cells. Tumor lysis was determined by loss of NIR signal quantification by IncuCyte (Sartorius) instrument and analysis, with images taken hourly. Several DCRs enhance lysis of Panc1 tumor cells, relative to control (FL).

FIG. 21. Proliferation quantification strategy. Engineered primary human CD8 T cells were labelled with CellTrace Violet (CTV, ThermoFisher) and unstimulated or stimulated at a 1:1 ratio with 1 ug/mL peptide_{MSLN}-pulsed HLA-A201-engineered K562 cells for 7 days. Cells were stained with specific antibodies and analyzed by flow cytometry. Gating strategy is shown for non-proliferated (CTVhi), intermediate proliferated (CTVint), and highly proliferated (CTVIo) T cells.

FIG. 22. Expression of 4-1BBL DCRs enhances proliferation relative to TCR-only T cells. Proliferation of 4-1BBL DCR-engineered T cells. Engineered primary human CD8 T cells were labelled with CellTrace Violet (CTV, ThermoFisher) and unstimulated or stimulated at a 1:1 ratio with 1 ug/mL peptide maN -pulsed HLA-A201-engineered K562 cells for 7 days, in triplicate. Cells were stained with specific antibodies and analyzed by flow cytometry. *P<0.05, students t test versus TCR only control.

FIG. 23. Expression of several OX40L DCRs enhances proliferation relative to TCR-only T cells. Proliferation of OX40L DCR-engineered T cells. Engineered primary human CD8 T cells were labelled with CellTrace Violet (CTV, ThermoFisher) and unstimulated or stimulated at a 1:1 ratio with 1 ug/mL peptide_{MSLN}-pulsed HLA-A201-engineered K562 cells for 7 days, in triplicate. Cells were stained with specific antibodies and analyzed by flow cytometry. *P<0.05, **P<0.01, ***P<0.001, students t test versus TCR only control.

FIG. 24. CD40L DCR expression in primary murine T cells. C57BL/6 splenocytes were transduced with a retroviral vector including the indicated transgene. T cells were stained with specific antibody to CD40L and analyzed by flow cytometry. GFP (green fluorescence protein) indicates an empty vector control. CD40L T cells exhibit surface expression in primary murine T cells.

FIG. 25. 4-1BBL DCR expression in primary murine T cells. C57BL/6 splenocytes were transduced with a retroviral vector including the indicated transgene. T cells were stained with specific antibody to 4-1BBL and analyzed by flow cytometry. 4-1BBL T cells exhibit surface expression in primary murine T cells.

FIG. 26. OX40L DCR expression in primary murine T cells. C57BL/6 splenocytes were transduced with a retroviral vector including the indicated transgene. T cells were stained with specific antibody to OX40L and analyzed by flow cytometry. OX40L T cells exhibit surface expression in primary murine T cells.

FIG. 27. Effective DCRs show strong accumulation in vitro relative to DCRneg T cells. Enrichment of transduced murine T cells in a mixed population including nontransduced T cells after 3 weekly cycles of stimulation with irradiated tumor cells and splenocytes. C57BL/6 splenocytes were transduced with a retroviral vector including the indicated transgene with a GFP expression gene linked by P2A. T cells were analyzed by flow cytometry. Effective DCR T cells outcompete nontransduced T cells and accumulate with multiple stimulations.

FIG. 28. CD40L DCRs enhance cytotoxic CD8+ T cell function. Lysis of FBL murine tumor cells by CD40L DCR-engineered primary murine TCRgag T cells. Engineered primary murine TCRgag CD8 T cells were cultured at a 1:1 ratio with NIR⁺ FBL cells. Tumor lysis was determined by loss of NIR signal as quantified by IncuCyte (Sartorius) instrument and analysis, with images taken hourly. Several DCRs enhance lysis of FBL tumor cells, relative to control (FL).

FIG. 29. DCRs enhance dendritic cell (DC) maturation and ability to stimulate T cells. DCs can present antigen and costimulatory ligands for T cell activation. THP-1 cells (monocyte cell line) were differentiated to immature DCs (immDCs) by 3-day treatment with 20 ng/mL IL-4+20 ng/mL PMA (phorbol 12-myristate 13-acetate). The ability of DCR T cells to induce DC maturation was tested by co-incubating DCR Jurkat T cells with immDCs for an additional two days. Control immDCs were differentiated to mature DCs (mDC) by 20 ng/mL IL-4+20 ng/mL PMA+1 ug/mL lipopolysaccharide (LPS) for 48 hours. Flow cytometry was performed to evaluate expression of human leukocyte antigen (HLA)—A, B,C, and CD80. DCR T cells induce maturation of DCs, promoting the ability of DCs to stimulate other anti-tumor T cells.

FIG. 30. OX40L DCR T cells exhibit enhanced expansion and accumulation in vivo. Murine TCR-T cells were engineered to express the indicated transgene and transferred to tumor-bearing mice. Mice were bled 7 days later and transferred T cells were determined by Thy1.1 expression as detected by flow cytometry. DCR T cells were at a higher concentration relative to control T cells (GFP, FL OX40L).

FIG. 31. OX40L DCR T cell therapy improves survival in immunocompetent murine AML model. Survival of tumor-bearing mice (FBL AML). B6 mice were injected with 4e6 FBL cells. Five days later, FBL-bearing mice were treated with Cytoxan (Cy) as a pre-conditioning regimen, and cohorts received 1e6 tumor-targeted (TCRgag) T cells i.p. Mice received no treatment (No Cy, n=2), Cy only (hashed black, No tcell, n=2), or Cy plus T cells engineered with one of the following: GFP only (control, GFP, n=4), full-length OX40L (FL OX40, n=2), or OX40L-CD27 (n=2), OX40L-CD40 (n=1). OX40L DCRs enhance survival relative to controls.

FIGS. 32A-32G. Exemplary sequences supporting the disclosure as follows:

(32A) full-length stimulatory and co-stimulatory immune cell proteins and ligands including 4-1BBL, mu41BBL, OX40L, muOX40L, CD40L, 4-1BB, OX40, CD27, CD30, CD40, huFLT3L, huLIGHT, HVEM, DR3, CD3ε, CD3δ, CD3 CD25, CD28, CD79a, CD79b, SLAMF1, ICOS, GITR, DAP10, DAP12, CD70, CD30L, TL1A, CARD11, FcR-γ, Fyn, LAT, LRP, NKG2D, NOTCH1, NOTCH2, NOTCH3, NOTCH4, ROR2, RYK, Slp76, pTα, TCRα, TCRβ, TRIM, ZAP70, PTCH2, CD2, CD226, CRTAM, TIM1, Ly9, CD84, SLAMF7, and BLAME;

(32B) exemplary extracellular domains of 41BBL; mu41BBL; OX40L; muOX40L; CD40L; muCD40L; 4-1BB; OX40; CD27; CD30; CD40; huFLT3L; huLIGHT; HVEM; DR3; CD3ε; CD3δ; CD3ζ; CD25; CD28; CD79a; CD79b; SLAMF1; ICOS; GITR; DAP10; and DAP12;

(32C) exemplary intracellular domains of 4-1BBL; mu41BBL; OX40L; muOX40L; CD40L; muCD40L; 41BB; OX40; CD27; CD30; CD40; huFLT3L; huLIGHT; HVEM; DR3; CD3ε; CD3δ; CD3ζ; CD25; CD28; CD79a; CD79b; SLAMF1; ICOS; GITR; DAP10; and DAP12;

(32D) exemplary transmembrane domains of 4-1BBL; mu41BBL; OX40L; muOX40L; CD40L; muCD40L; 4-1BB; OX40; CD27; CD30; CD40; huFLT3L; huLIGHT; HVEM; DR3; CD3ε; CD3δ; CD3ζ; CD25; CD28; CD79a; CD79b; SLAMF1; ICOS; GITR; DAP10; DAP12; and CD47;

(32E) truncated (tr) forms of 4-1BBL, mu41BBL, OX40L, muOX40L, CD40L, muCD40L, huFLT3L, and huLIGHT. These truncated proteins include a methionine followed by a transmembrane domain and extracellular domain;

(32F) exemplary hybrid proteins disclosed herein. In these descriptions, Met stands for Methionine, ICD stands for intracellular domain, TM stands for transmembrane domain, and ECD stands for extracellular domain:

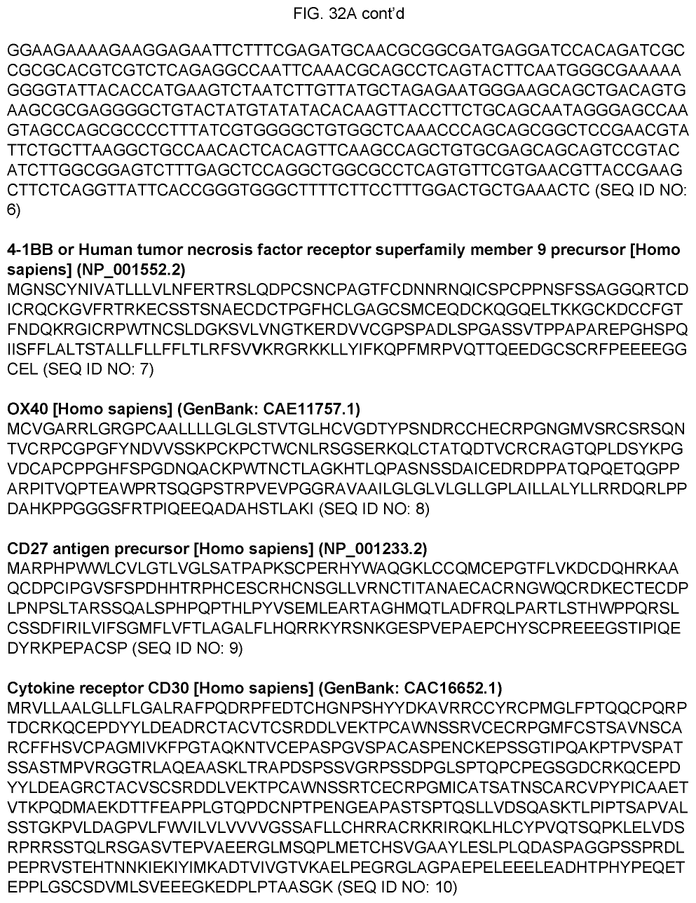

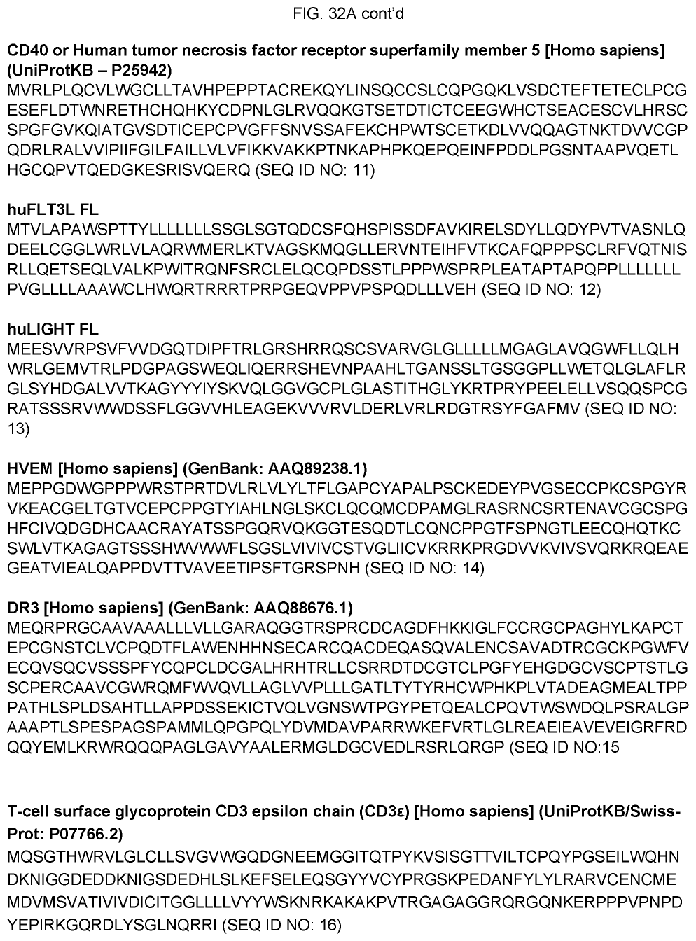

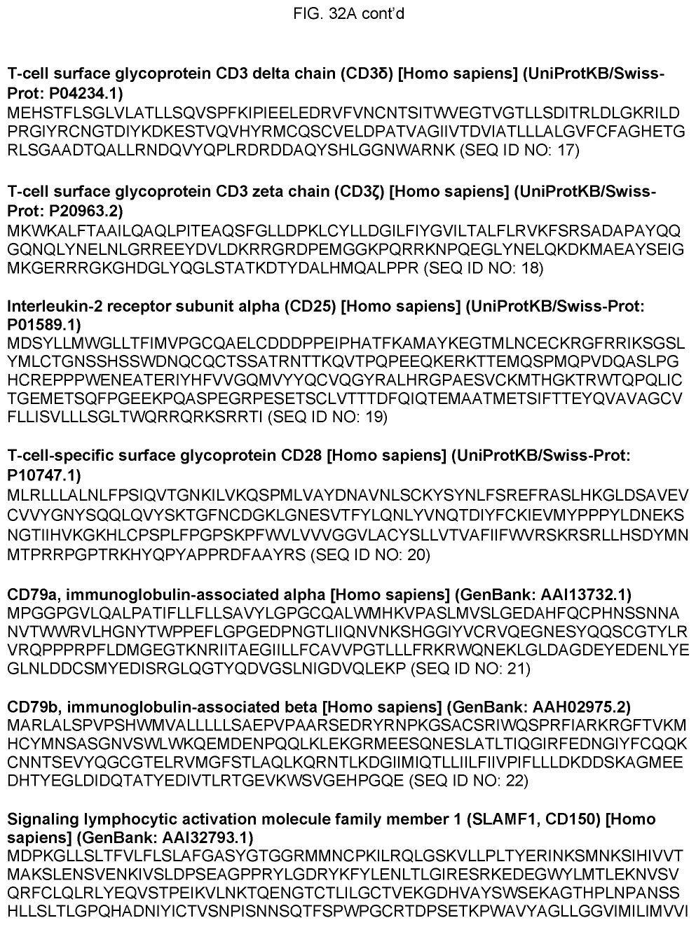

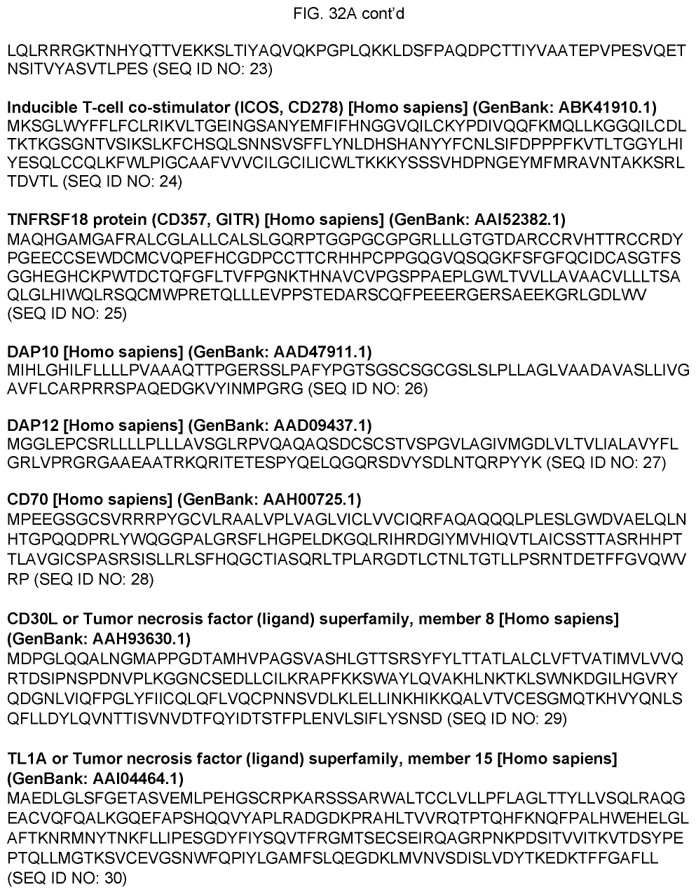

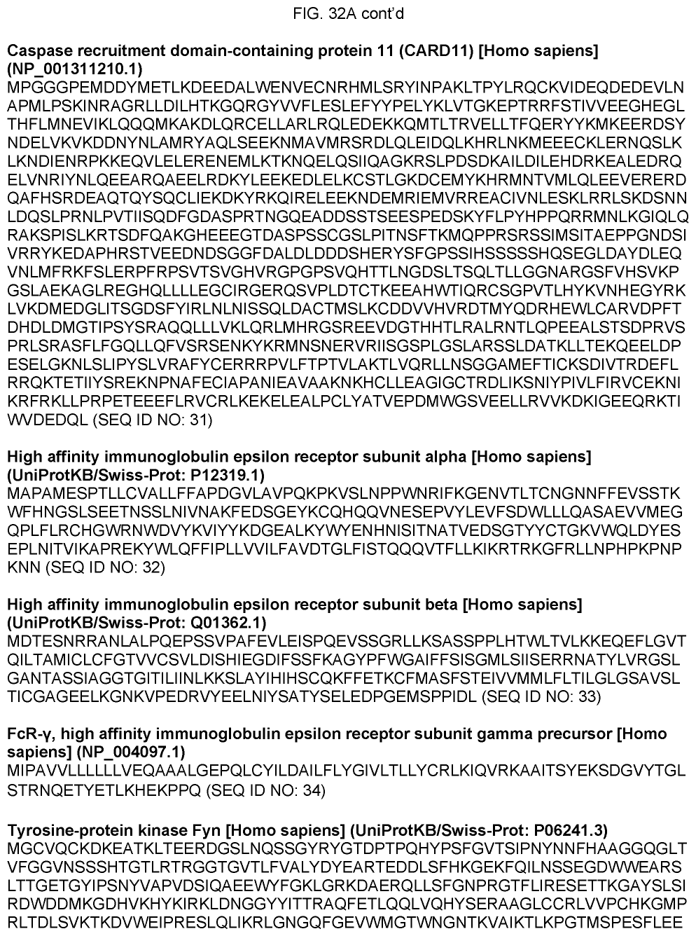

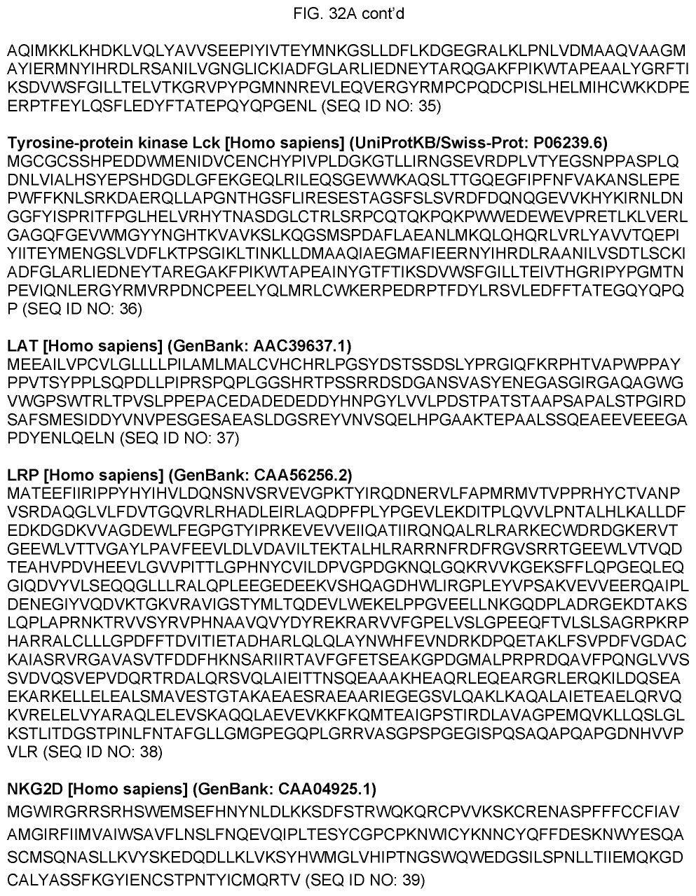

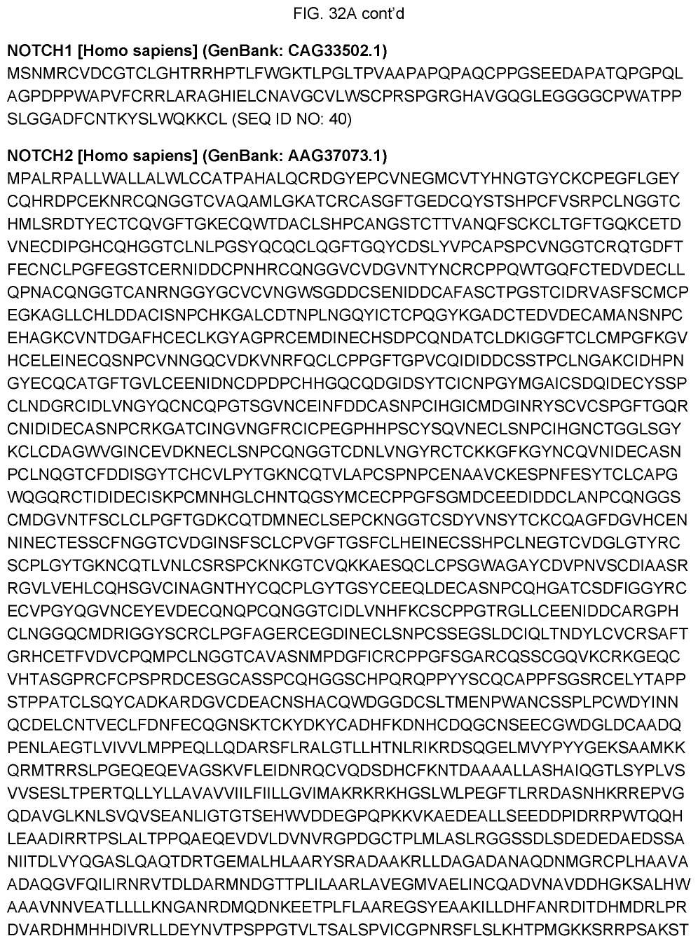

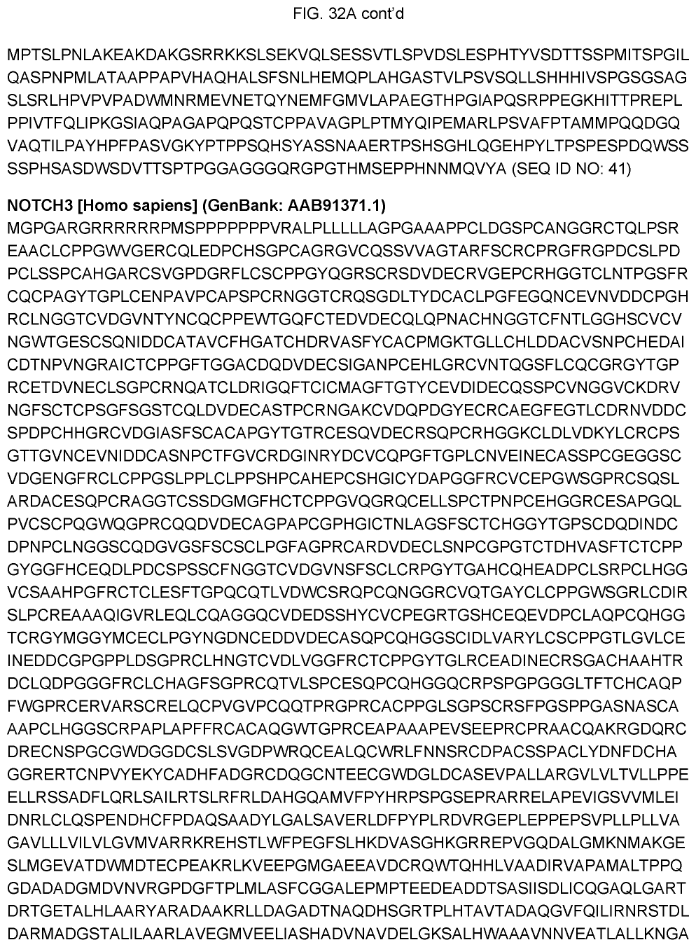

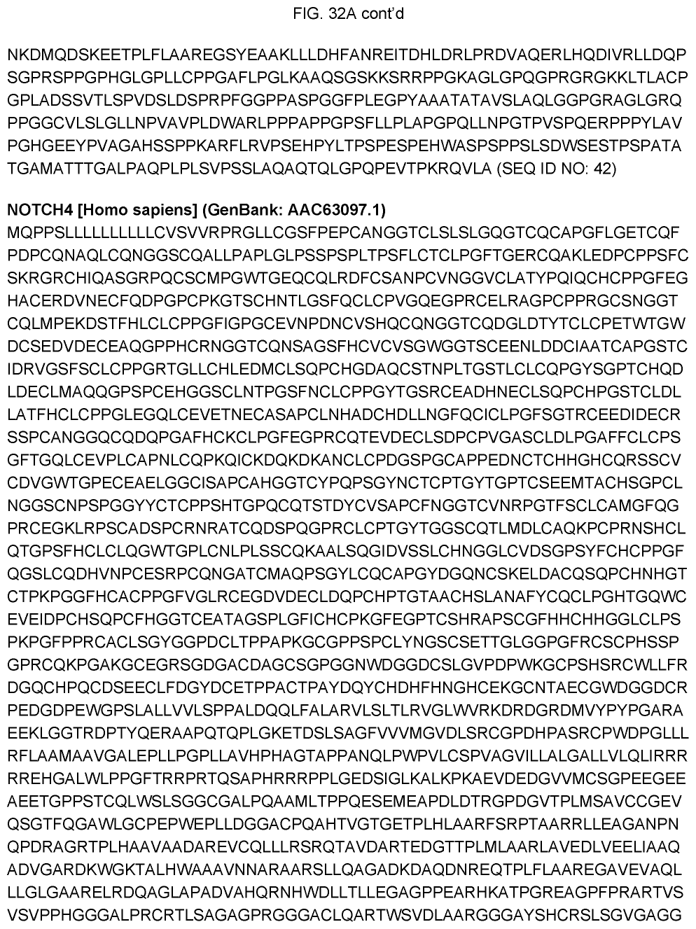

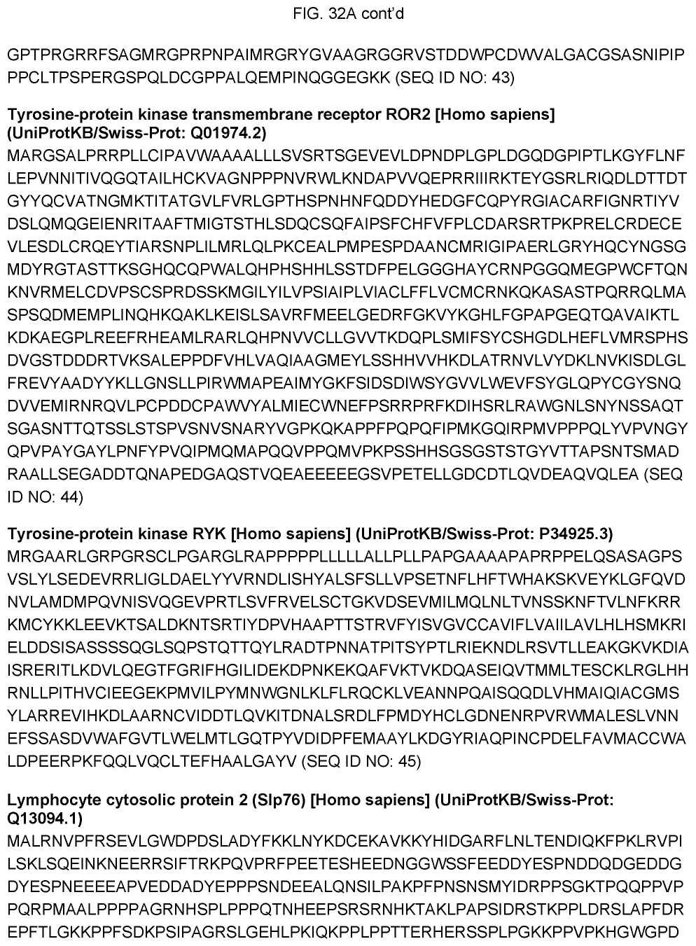

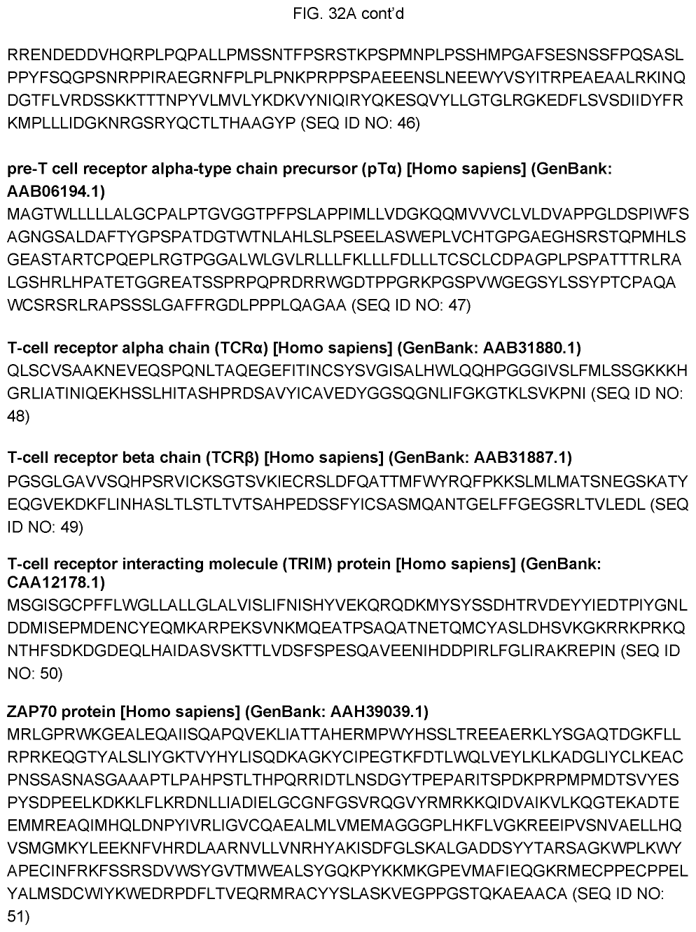

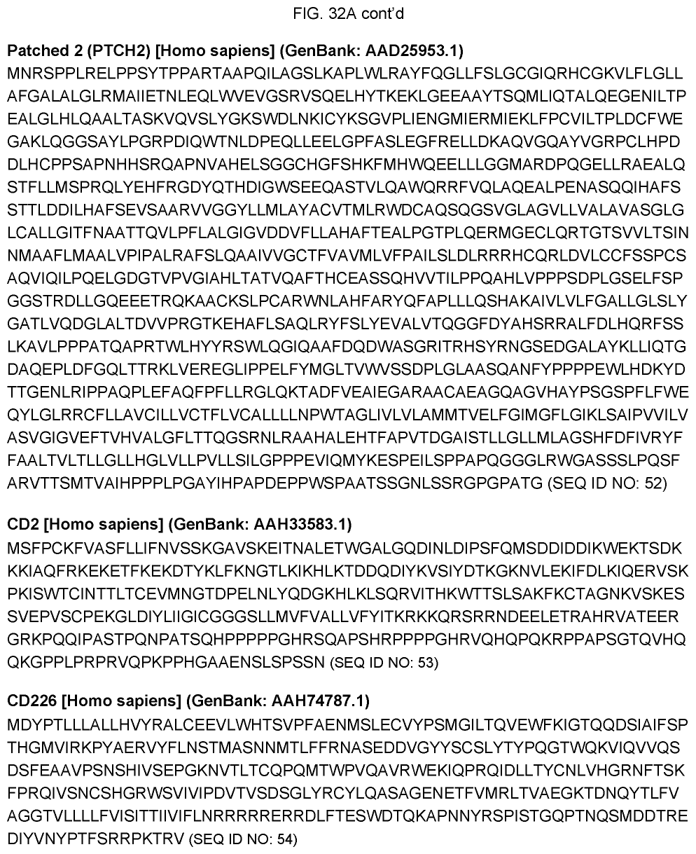

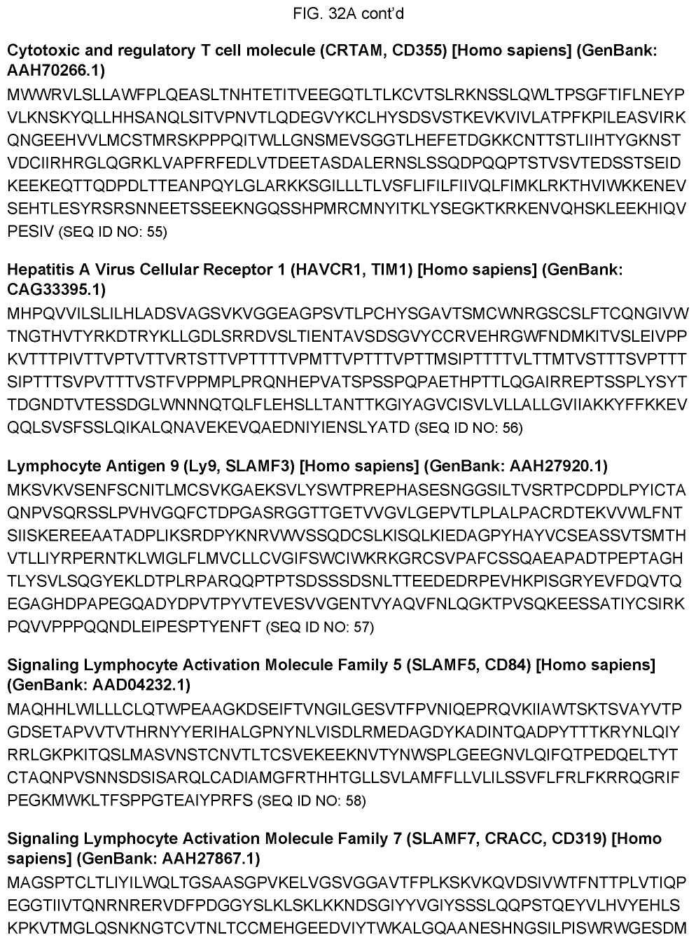

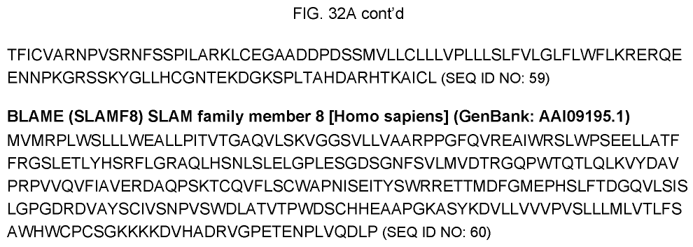

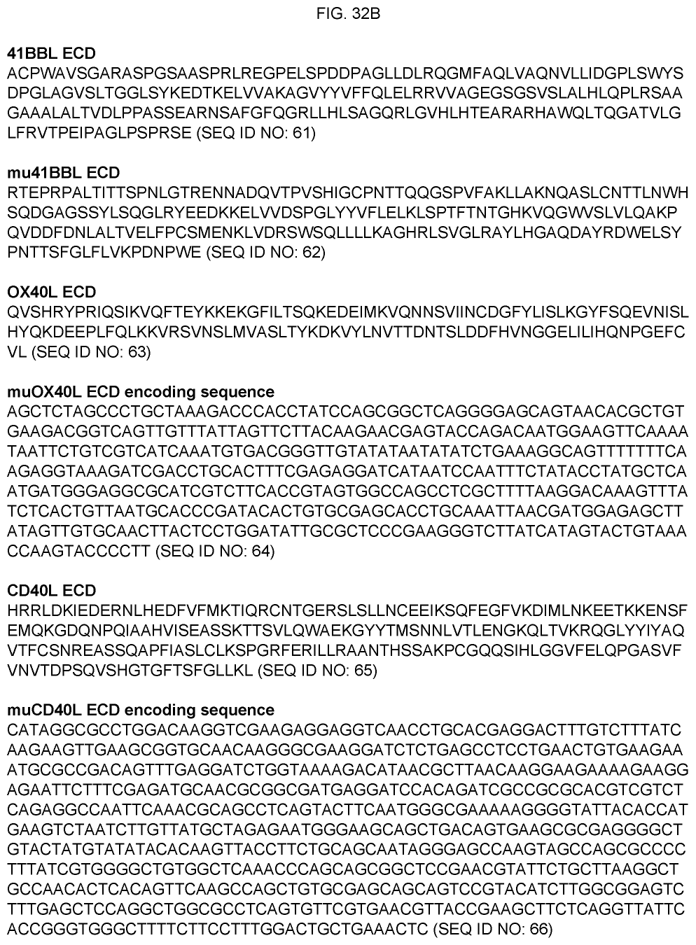

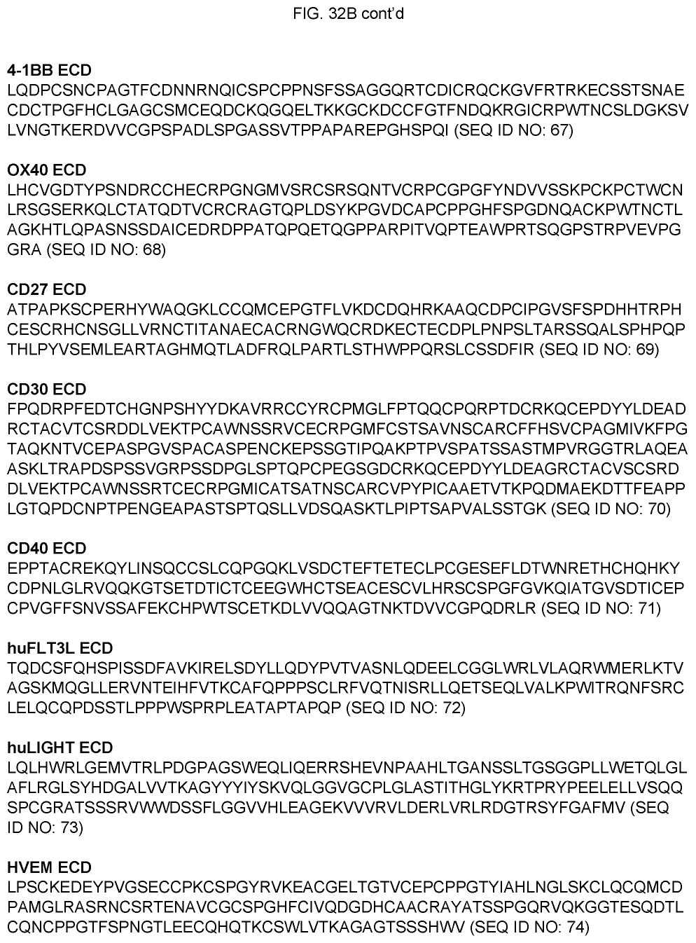

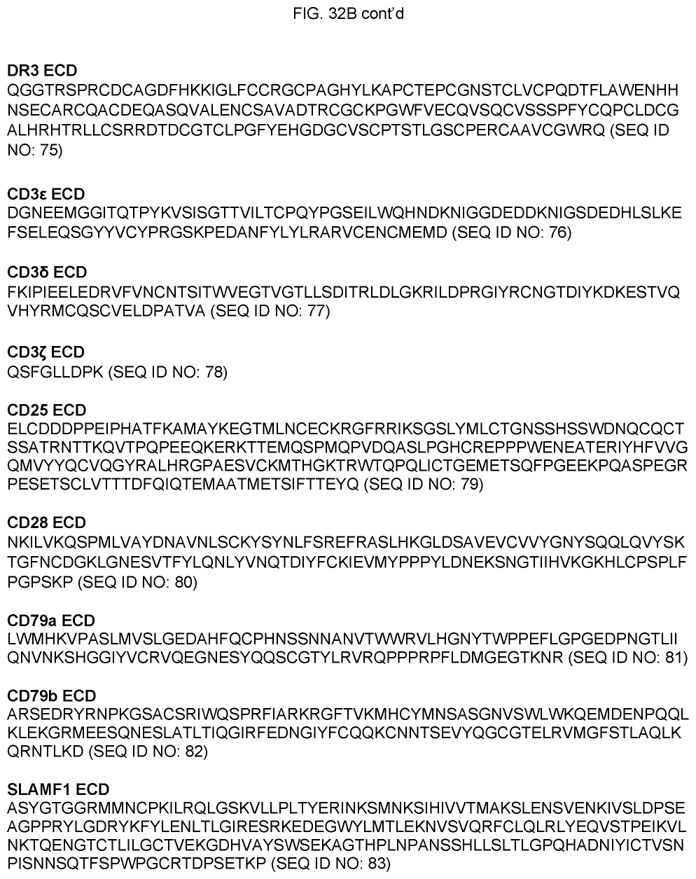

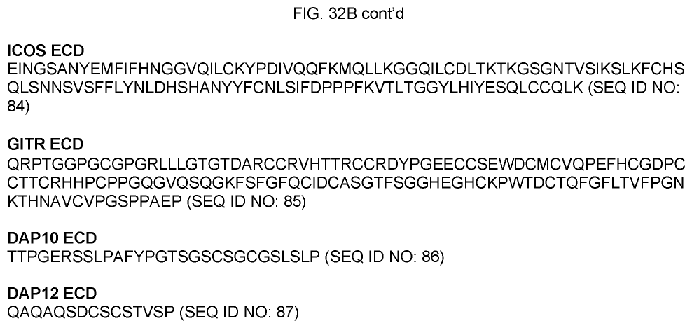

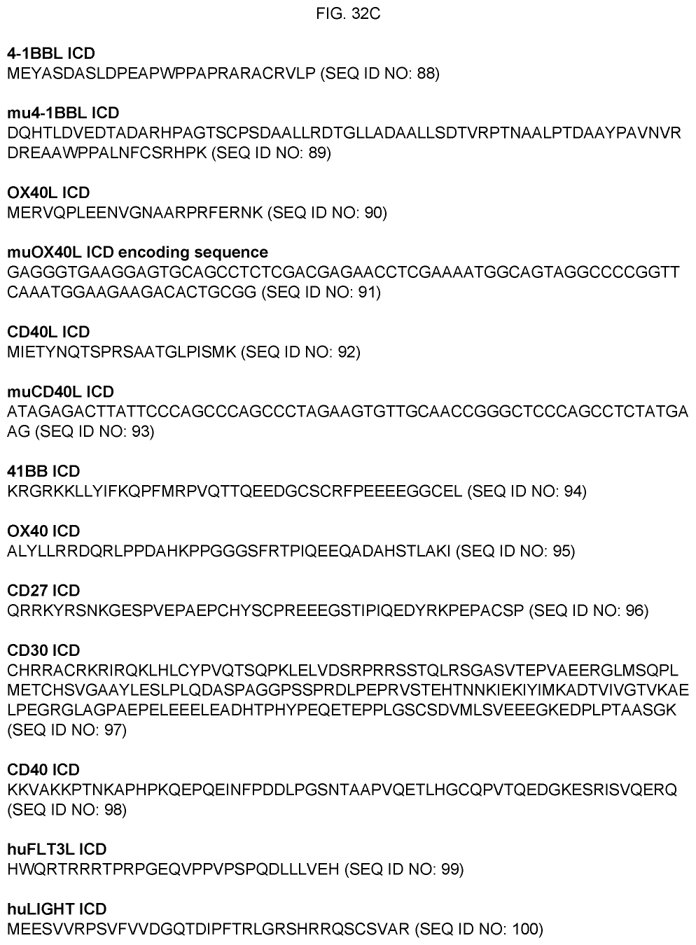

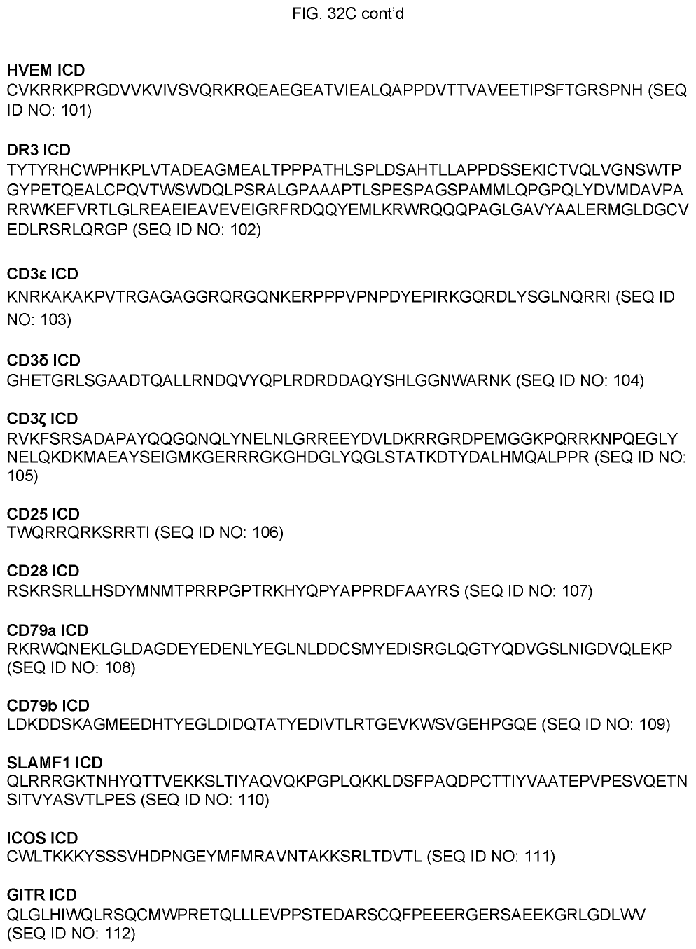

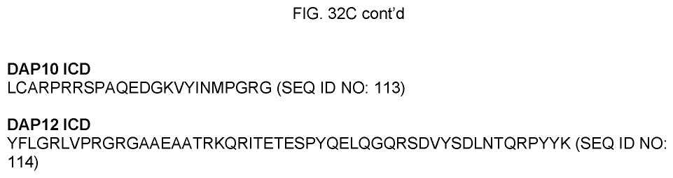

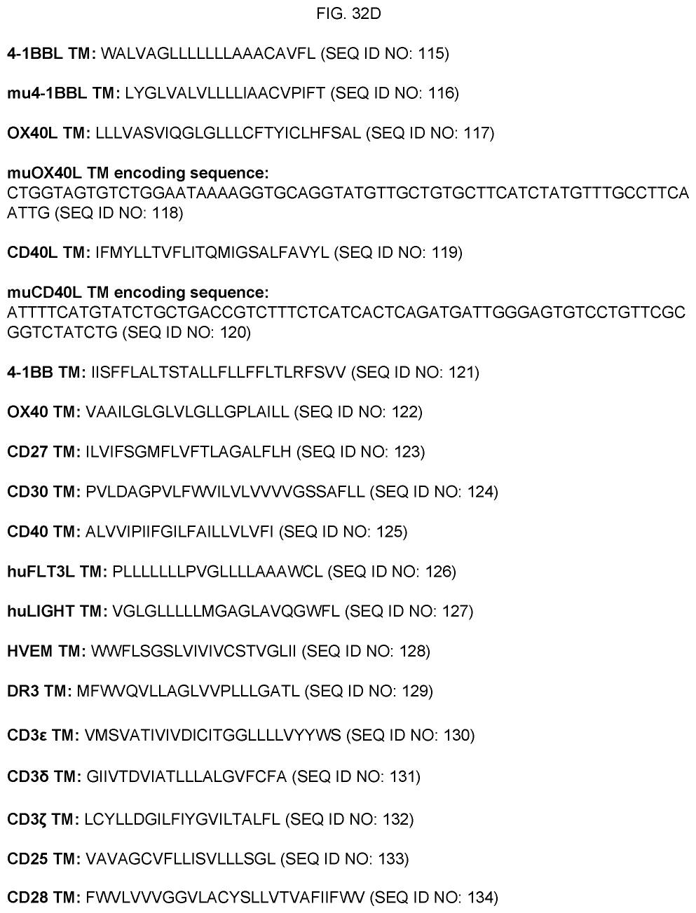

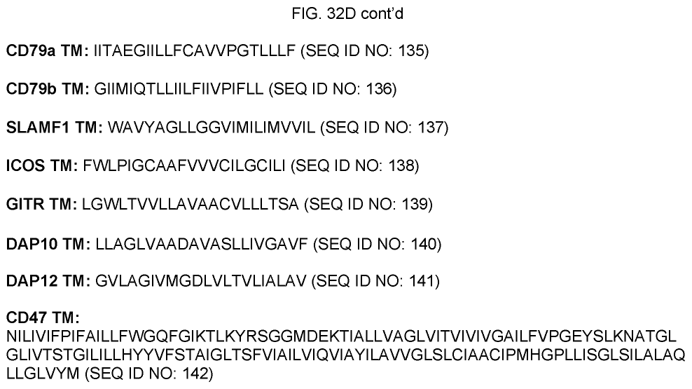

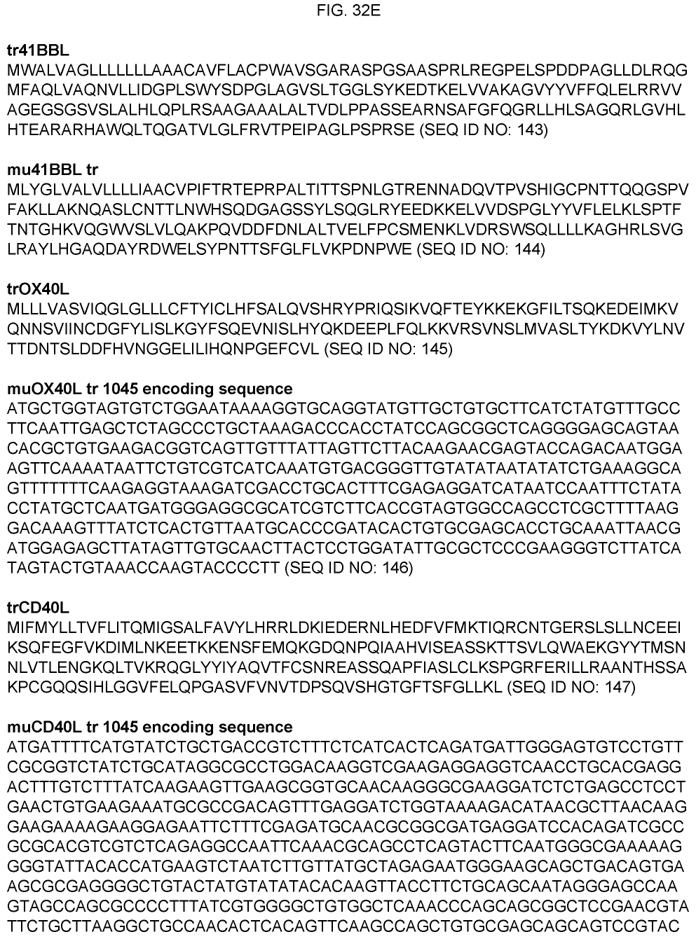

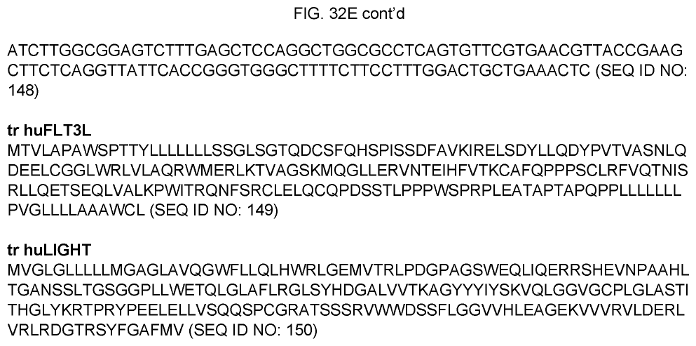

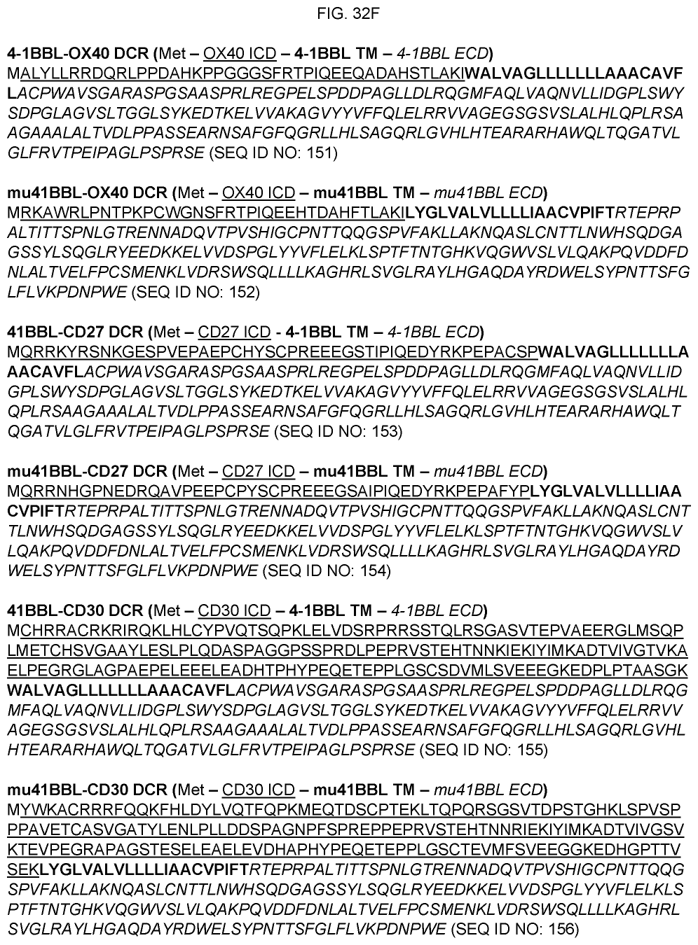

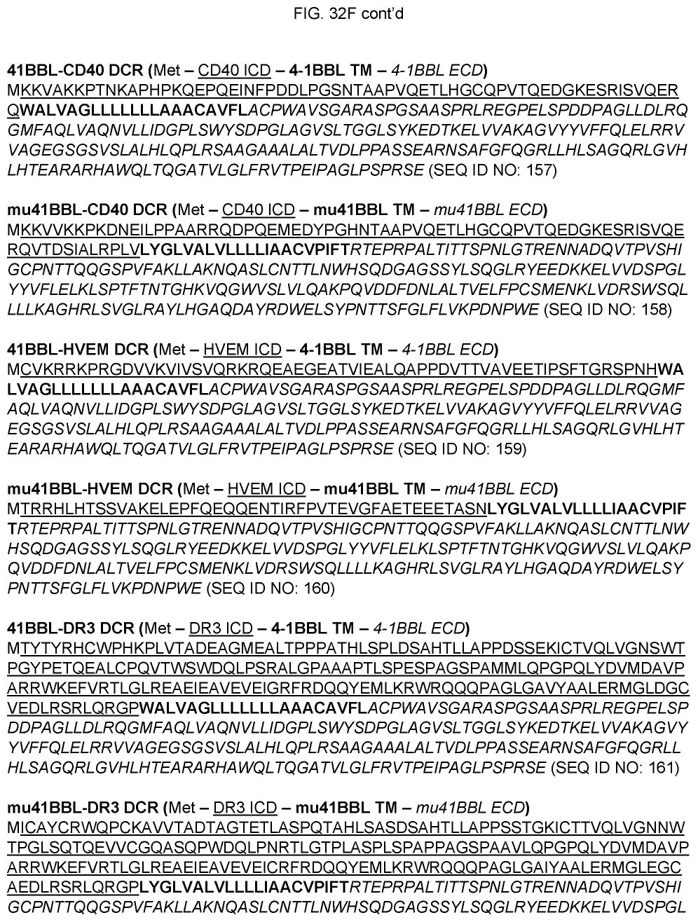

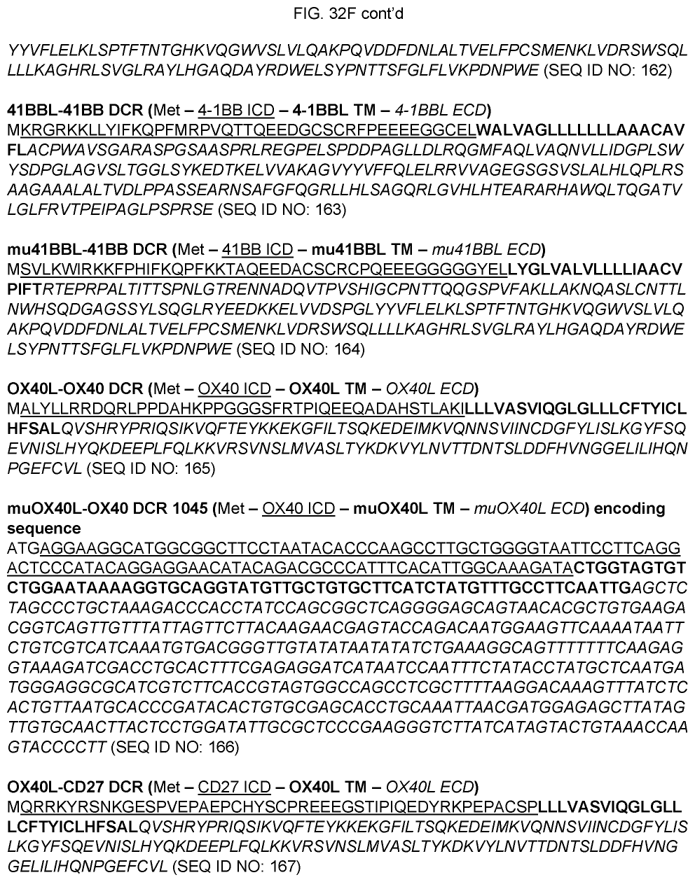

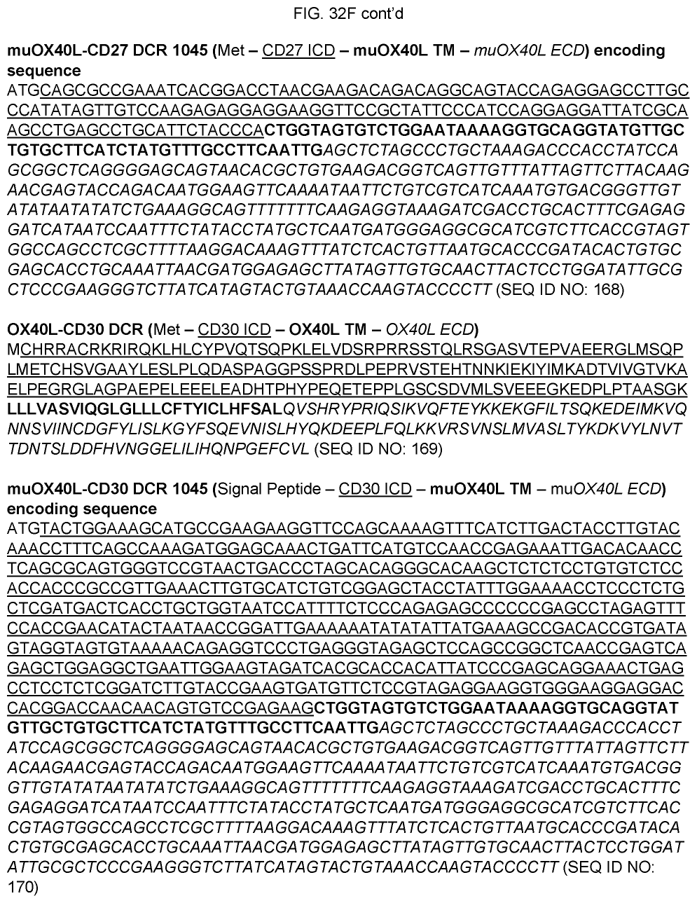

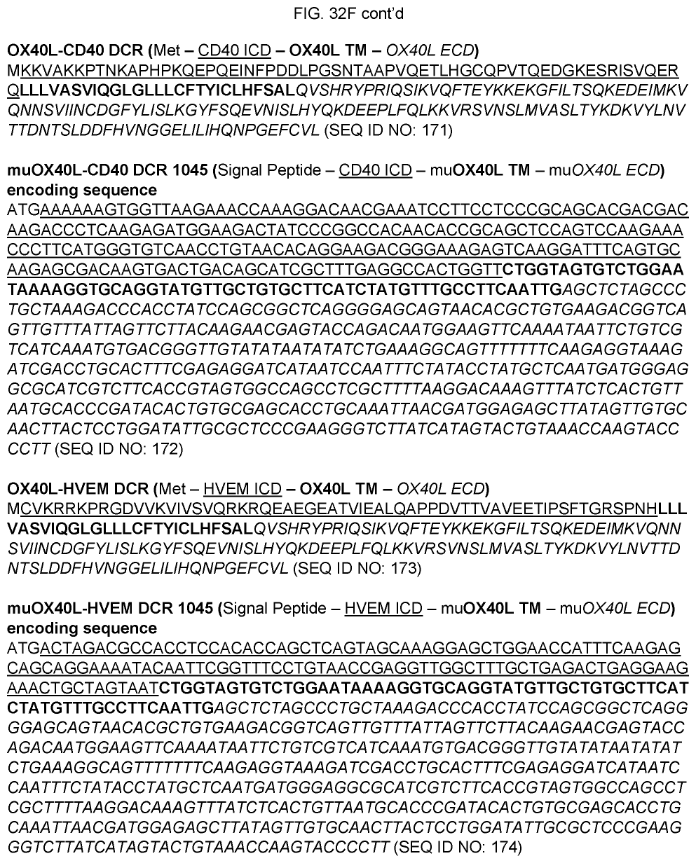

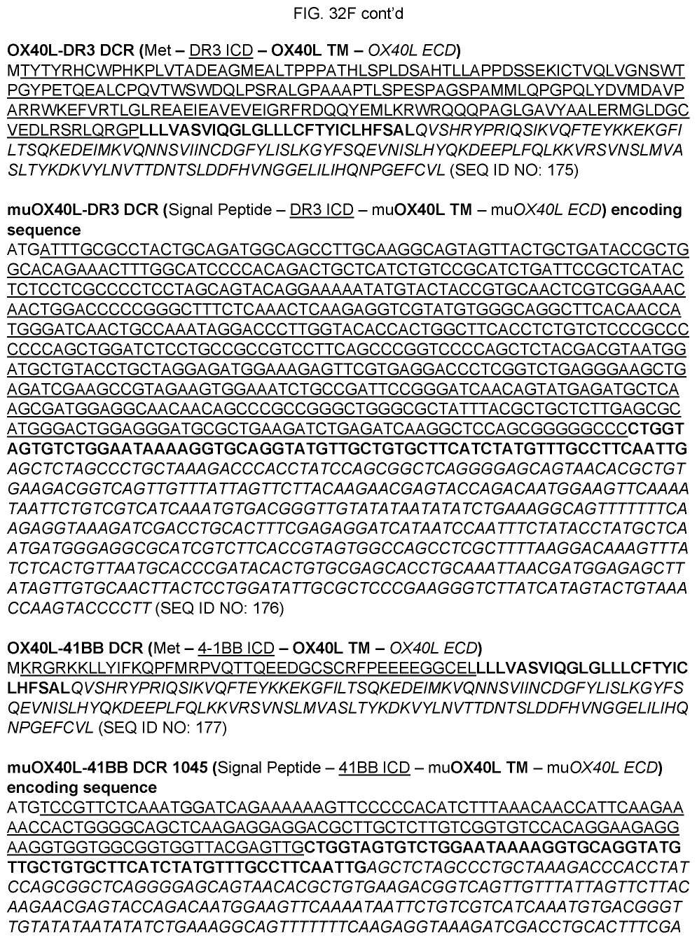

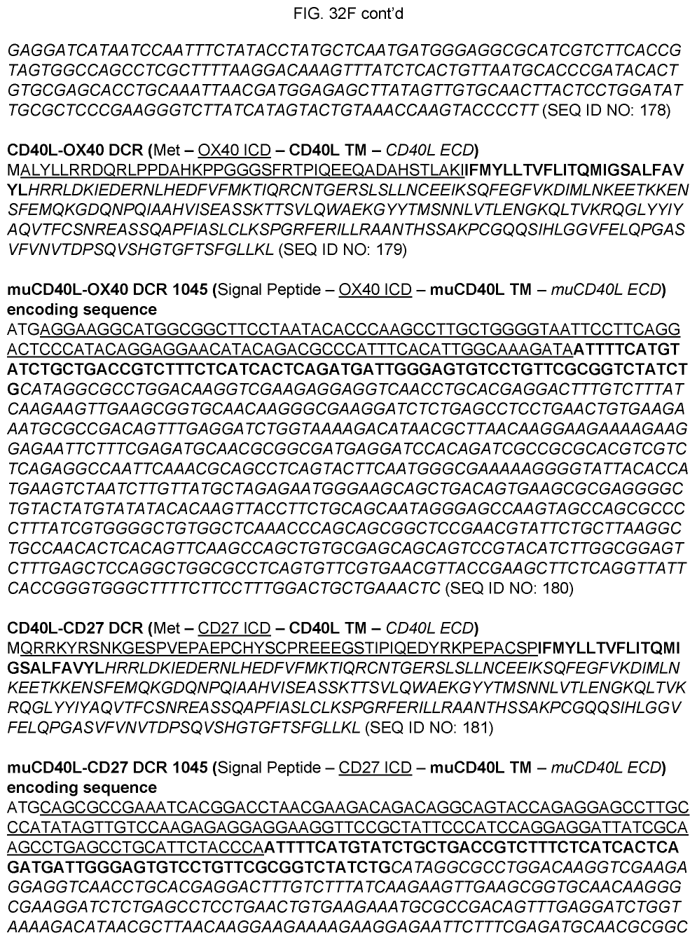

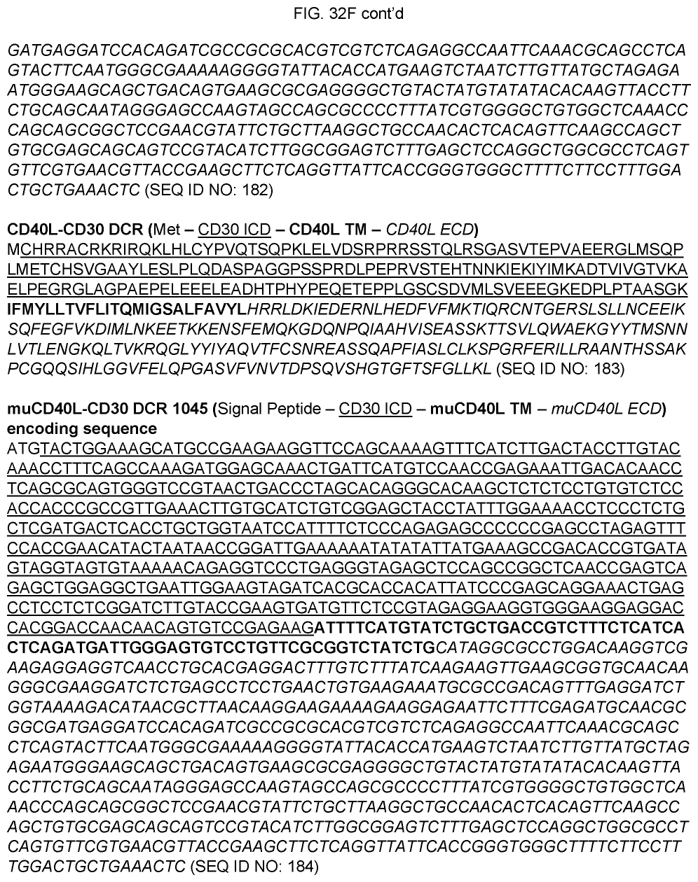

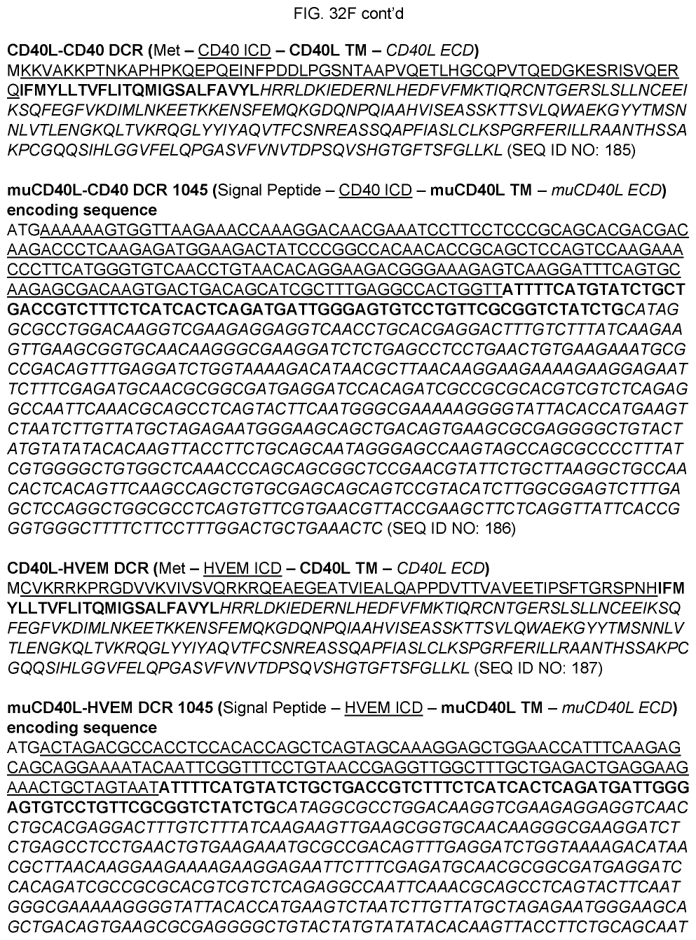

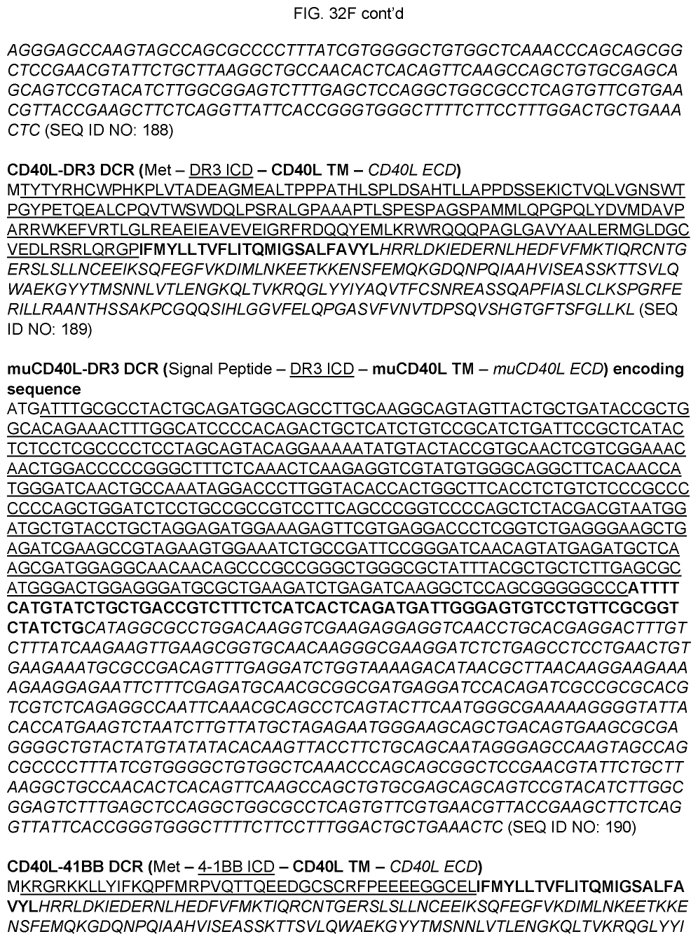

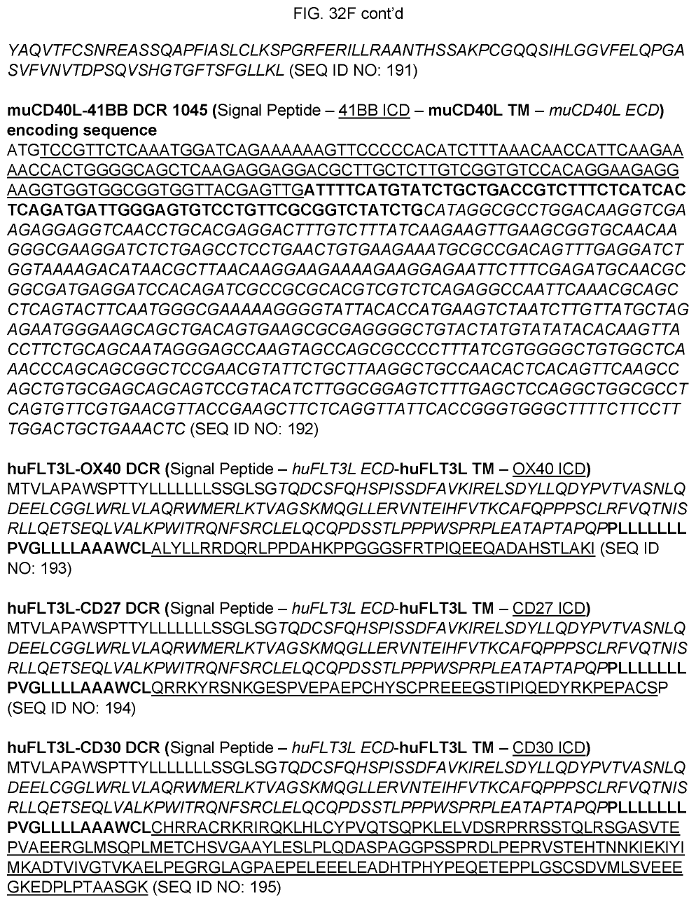

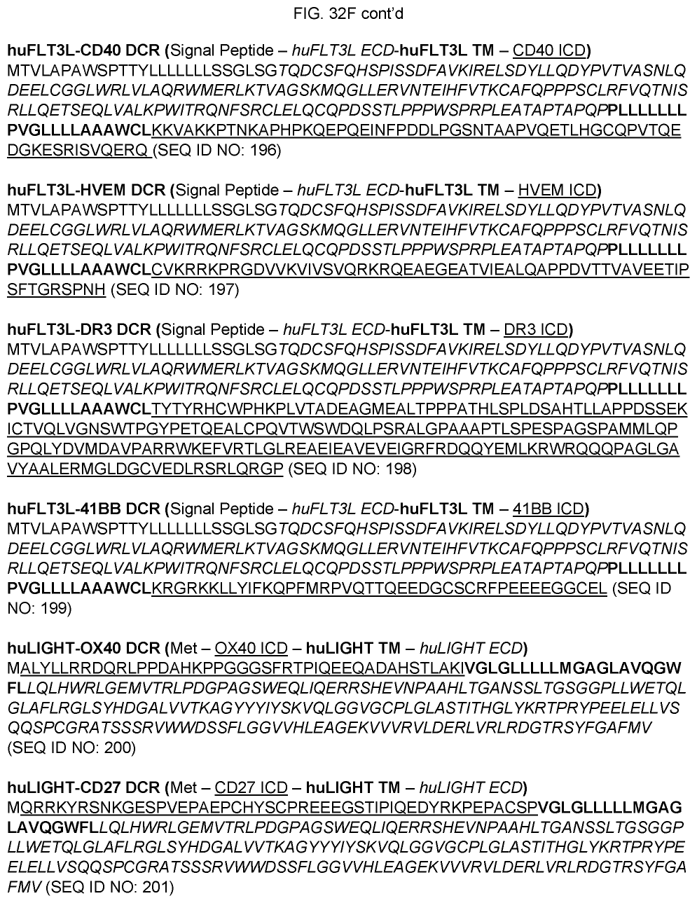

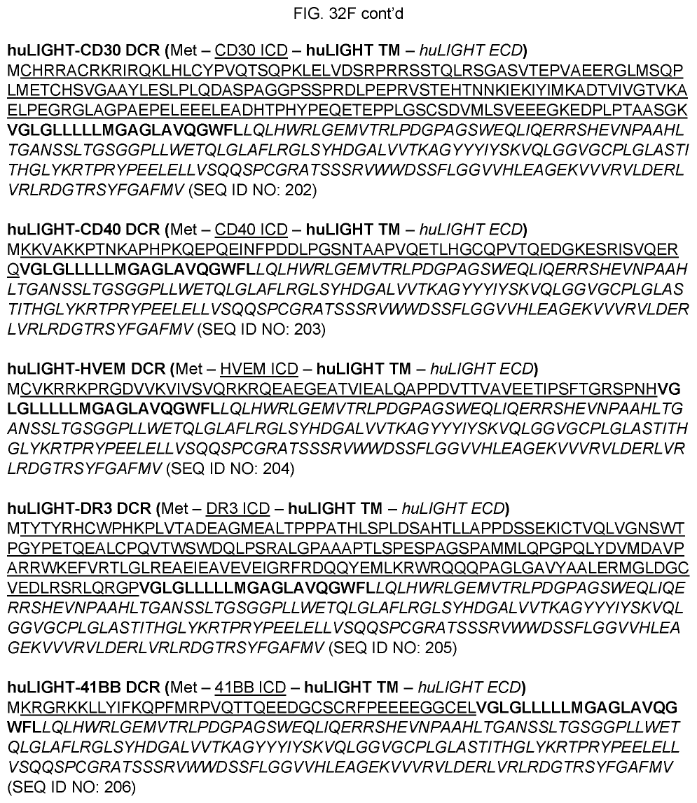

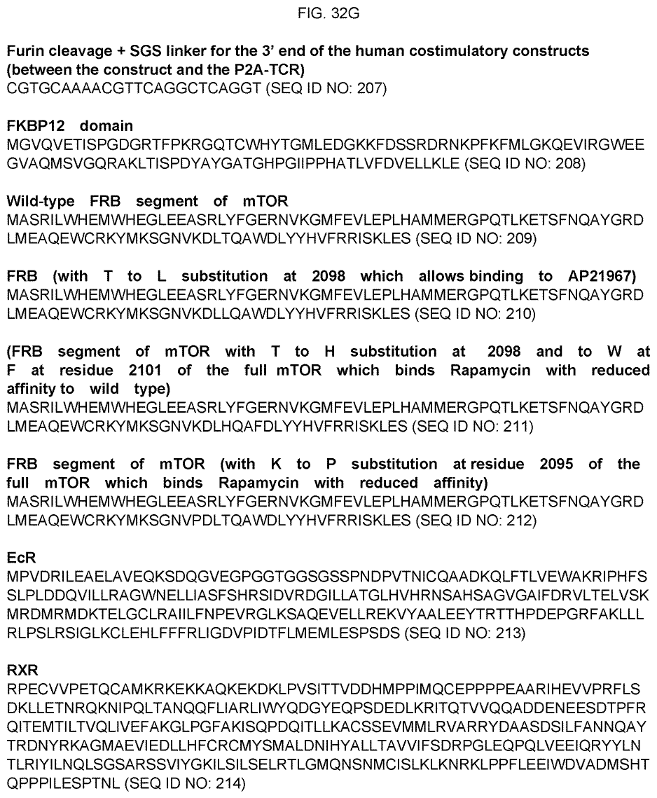

4-1BBL-OX40 hybrid (269 aa; Met—OX40 ICD—4-1BBL TM—4-1BBL ECD);
mu41BBL-OX40 hybrid (264 aa; Met—OX40 ICD—mu41BBL TM—mu41BBL ECD);
41BBL-CD27 hybrid (275 aa; Met—CD27 ICD—4-1BBL TM—4-1BBL ECD);
mu41BBL-CD27 hybrid (275 aa; Met—CD27 ICD—mu41BBL TM—mu41BBL ECD);
41BBL-CD30 hybrid (269 aa; Met—CD30 ICD—4-1BBL TM—4-1BBL ECD);
mu41BBL-CD30 hybrid (418 aa; Met—CD30 ICD—mu41BBL TM—mu41BBL ECD);
41BBL-CD40 hybrid (289 aa; Met—CD40 ICD—4-1BBL TM—4-1BBL ECD);
mu41BBL-CD40 hybrid (302aa; Met—CD40 ICD—mu41BBL TM—mu41BBL ECD);
41BBL-HVEM hybrid (287 aa; Met—HVEM ICD—4-1BBL TM—4-1BBL ECD);
mu41BBL-HVEM hybrid (272aa; Met—HVEM ICD—mu41BBL TM—mu41BBL ECD);
41BBL-DR3 hybrid (424 aa; Met—DR3 ICD—4-1BBL TM—4-1BBL ECD);
mu41BBL-DR3 hybrid (426aa; Met—DR3 ICD—mu41BBL TM—mu41BBL ECD);
41BBL-41BB hybrid (269 aa; Met—4-1BB ICD—4-1BBL TM—4-1BBL ECD);
mu41BBL-41BB hybrid (273aa; Met—41BB ICD—mu41BBL TM—mu41BBL ECD);
OX40L-OX40 hybrid (203 aa; Met—OX40 ICD—OX40L TM—OX40L ECD);
muOX40L-OX40 hybrid (621 nt; Met—OX40 ICD—muOX40L TM—muOX40L ECD);
OX40L-CD27 hybrid (209 aa; Met—CD27 ICD—OX40L TM—OX40L ECD);
muOX40L-CD27 hybrid (654 nt; Met—CD27 ICD—muOX40L TM—muOX40L ECD);
OX40L-CD30 hybrid (350 aa; Met—CD30 ICD—OX40L TM—OX40L ECD);
muOX40L-CD30 hybrid (1,083 nt; Signal Peptide—CD30 ICD—muOX40L TM—muOX40L ECD)
OX40L-CD40 hybrid (223 aa; Met—CD40 ICD—OX40L TM—OX40L ECD);
muOX40L-CD40 hybrid (735 nt; Signal Peptide—CD40 ICD—muOX40L TM—muOX40L ECD);
OX40L-HVEM hybrid (221 aa; Met—HVEM ICD—OX40L TM—OX40L ECD);
muOX40L-HVEM hybrid (645 nt; Signal Peptide—HVEM ICD—muOX40L TM—muOX40L ECD),
OX40L-DR3 hybrid (358 aa; Met—DR3 ICD—OX40L TM—OX40L ECD);
muOX40L-DR3 hybrid (1,107 nt; Signal Peptide—DR3 ICD—muOX40L TM—muOX40L ECD);
OX40L-41BB hybrid (203 aa; Met—4-1BB ICD—OX40L TM—OX40L ECD);
muOX40L-41BB hybrid (657 nt; Signal Peptide—41BB ICD—muOX40L TM—muOX40L ECD);
CD40L-OX40 hybrid (282 aa; Met—OX40 ICD—CD40L TM—CD40L ECD);
muCD40L-OX40 hybrid (825 nt; Signal Peptide—OX40 ICD—muCD40L TM—muCD40L ECD);
CD40L-CD27 hybrid (288 aa; Met—CD27 ICD—CD40L TM—CD40L ECD);
muCD40L-CD27 hybrid (858 nt; Signal Peptide—CD27 ICD—muCD40L TM—muCD40L ECD);
CD40L-CD30 hybrid (429 aa; Met—CD30 ICD—CD40L TM—CD40L ECD);
muCD40L-CD30 hybrid (1,287 nt; Signal Peptide—CD30 ICD—muCD40L TM—muCD40L ECD);
CD40L-CD40 hybrid (302 aa; Met—CD40 ICD—CD40L TM—CD40L ECD);
muCD40L-CD40 hybrid (939 nt; Signal Peptide—CD40 ICD—muCD40L TM—muCD40L ECD);
CD40L-HVEM hybrid (300 aa; Met—HVEM ICD—CD40L TM—CD40L ECD);
muCD40L-HVEM hybrid (849 nt; Signal Peptide—HVEM ICD—muCD40L TM—muCD40LECD);
CD40L-DR3 hybrid (437 aa; Met—DR3 ICD—CD40L TM—CD40L ECD); and
muCD40L-DR3 hybrid (1,311 nt; Signal Peptide—DR3 ICD—muCD40L TM—muCD40L ECD);
CD40L-41BB hybrid (282 aa; Met—4-1BB ICD—CD40L TM—CD40L ECD);
muCD40L-41BB hybrid (861 nt; Signal Peptide—41BB ICD—muCD40L TM—muCD40L ECD);
huFLT3L-OX40 hybrid (247aa; Signal Peptide—huFLT3L ECD-huFLT3L TM—OX40 ICD);
huFLT3L-CD27 hybrid (253 aa; Signal Peptide—huFLT3L ECD-huFLT3L TM—CD27 ICD);
huFLT3L-CD30 hybrid (394 aa; Signal Peptide—huFLT3L ECD-huFLT3L TM—CD30 ICD);
huFLT3L-CD40 hybrid (267 aa; Signal Peptide—huFLT3L ECD-huFLT3L TM—CD40 ICD);
huFLT3L-HVEM hybrid (265 aa; Signal Peptide—huFLT3L ECD-huFLT3L TM—HVEM ICD);
huFLT3L-DR3 hybrid (402 aa; Signal Peptide—huFLT3L ECD-huFLT3L TM—DR3 ICD);
huFLT3L-41BB hybrid (247 aa; Signal Peptide—huFLT3L ECD-huFLT3L TM—41BB ICD);
huLIGHT-OX40 hybrid (246 aa; Met—OX40 ICD—huLIGHT TM—huLIGHT ECD);
huLIGHT-CD27 hybrid (252 aa; Met—CD27 ICD—huLIGHT TM—huLIGHT ECD);
huLIGHT-CD30 hybrid (393 aa; Met—CD30 ICD—huLIGHT TM—huLIGHT ECD);
huLIGHT-CD40 hybrid (266 aa; Met—CD40 ICD—huLIGHT TM—huLIGHT ECD);
huLIGHT-HVEM hybrid (264 aa; Met—HVEM ICD—huLIGHT TM—huLIGHT ECD);
huLIGHT-DR3 hybrid (401 aa; Met—DR3 ICD—huLIGHT TM—huLIGHT ECD); and
huLIGHT-41BB hybrid (246 aa; Met—41BB ICD—huLIGHT TM—huLIGHT ECD);
Within FIG. 32F, ICDs are underlined, transmembrane regions are in bold and ECDs are italicized. The signal methionines are in plain text; and

(32G) additional sequences supporting the disclosure including furin cleavage+SGS linker for the 3′ end of the human costimulatory constructs (between the construct and the P2A-TCR); FKBP12 domain; Wild-type FRB segment of mTOR; FRB (with T to L substitution at 2098 which allows binding to AP21967); (FRB segment of mTOR with T to H substitution at 2098 and to W at F at residue 2101 of the full mTOR which binds Rapamycin with reduced affinity to wild type); FRB segment of mTOR (with K to P substitution at residue 2095 of the full mTOR which binds Rapamycin with reduced affinity); EcR; and RXR.

### DETAILED DESCRIPTION

The immune system uses two general mechanisms to protect the body against cancerous cells or environmental pathogens such as viruses, bacteria, and fungi. One is the non-specific (or innate) inflammatory response. The other is the specific (acquired or adaptive) immune response. In contrast to the innate response which is fundamentally the same for each injury, acquired responses are custom tailored to particular cancers or pathogens.

The immune system can recognize and respond to differences between healthy/self and unhealthy/non-self-antigen, including antigens on cancerous cells. Acquired immunity has specific “memory” for these recognized antigens, and repeated exposure to the same antigen increases the response increases the level of induced protection against these previously-encountered cancers or pathogens.

Acquired immunity is mediated by the specialized immune cells, B and T lymphocytes. B lymphocytes produce and mediate their functions through the actions of antibodies. B lymphocyte dependent immune responses are referred to as “humoral immunity” because antibodies are detected in body fluids. T lymphocyte dependent immune responses are referred to as “cell mediated immunity” because effector activities are mediated directly by the local actions of effector T lymphocytes. The local actions of effector T lymphocytes are amplified through synergistic interactions between T lymphocytes and secondary effector cells, such as activated macrophages.

In order for immune cells to respond to foreign antigens, two signals must be provided by antigen-presenting cells (APCs) to resting T lymphocytes. The first signal, which confers specificity to the immune response, is transduced via the T cell receptor (TCR) when the TCR engages a specific peptide presented in the context of the major histocompatibility complex (MHC) (Rossy et al., Frontiers in Immunol. 3: 1-12, 2012). The second signal, or co-stimulatory signal, is an antigen-independent co-signal that directs T cell function and T cell fate (Lenschow et al. (1996) Annu. Rev. Immunol. 14:233) (Chen and Flies Nat Rev Imm 2013). A co-stimulatory signal can be provided by temporary binding to one or more distinct cell surface polypeptides expressed by APCs (Jenkins, M. K. et al. (1988) J. Immunol. 140:3324-3330).

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR).

Clinical trials with CAR-expressing T cells (CAR-T) have shown positive responses and cancer reduction in some patients. However, in others they have failed to provide prolonged anti-cancer activity. One reason for a lack of prolonged success may be that the T cells do not receive strong or diverse enough activation signals when bound to a targeted cell.

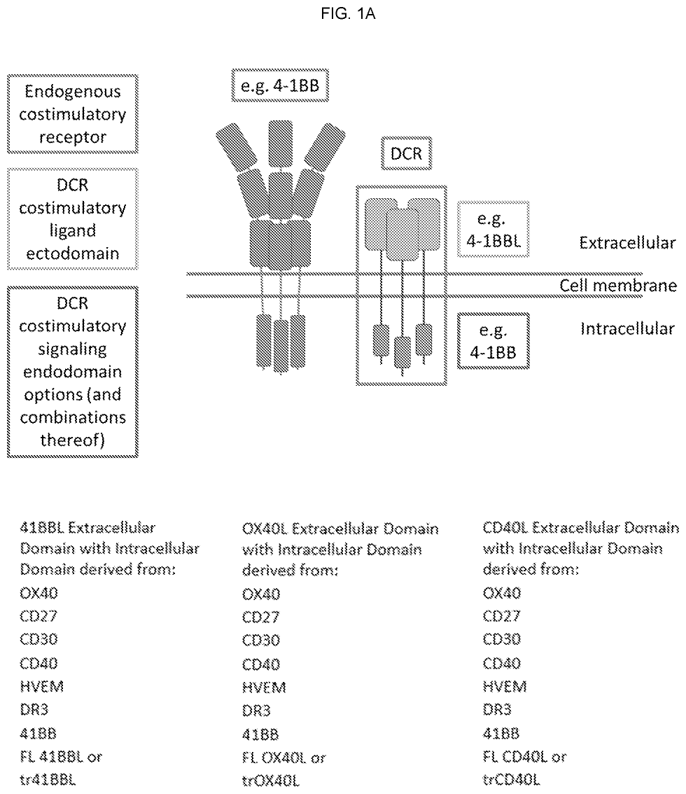

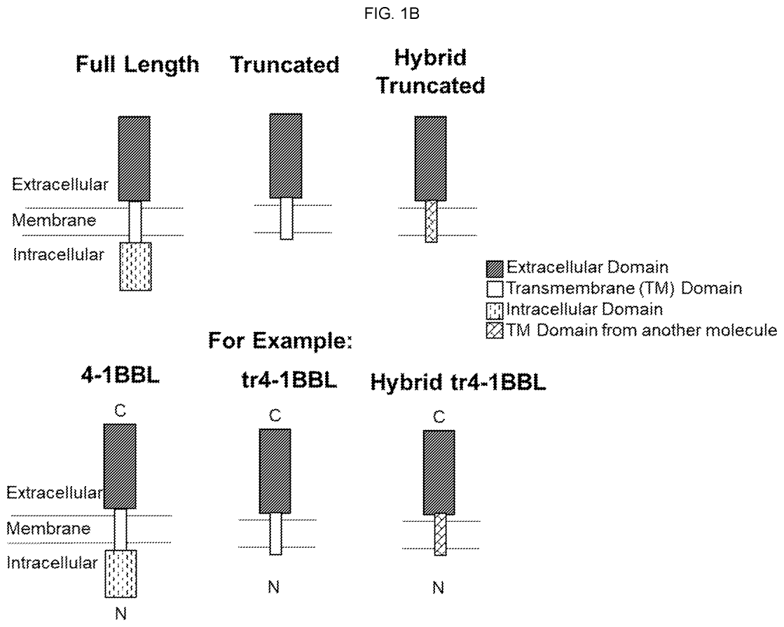

The current disclosure provides hybrid and truncated proteins that modulate and diversify immune cell activation. The hybrid proteins are stimulatory and include an extracellular domain of one stimulatory immune cell protein, at least one intracellular domain of one or more different stimulatory immune cell proteins, and a transmembrane domain linking the extracellular domain to the intracellular domain(s) (see, e.g., FIG. 1A). When expressed by immune cells, the disclosed hybrid proteins allow at least two stimulatory signals to be activated in the presence of a particular ligand, as explained more fully below.

Immune cell proteins that can be used to create hybrid stimulatory proteins described herein include 4-1BB (CD137), 4-1BBL (CD137L), OX40 (CD134), OX40L (CD134L), CD40, CD40L, CD30, CD30L, CD27, CD27L, DR3, DR3L, HVEM, LIGHT, SLAM F1 (CD150 or SLAM), SLAMF1L, ICOS (CD278), ICOSL, GITR (CD357), GITRL, CD122, CD132, CD28, CD80, CD86, CRTAM (CD355), CD79A, CD79AL, CD79B, CD79BL, CD84 (SLAMF5), CD226, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, Ly9 (SLAMF3), NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, SLAMF7 (CRACC or CD319), Slp76, Slp76L, pTα, pTαL, TCRα, TCRβ, HAVCR1 (TIM1), TIM1L, TRIM, TRIML, Zap70, Zap70L, PTCH2, PTCH2L, CD3ε; CD3δ; and CD3ζ. The L (ligand) designation refers to a stimulatory protein's natural ligand and functional variants and fragments thereof. Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in a binding study between a co-stimulatory protein and its associated natural ligand.

In some examples, hybrid proteins are formed from a co-stimulatory protein such as 4-1BB, 4-1BBL, OX40, OX40L, CD40, CD40L, CD30, CD30L, CD27, CD27L, DR3, DR3L, FLT3, FLT3L, LIGHT, or HVEM. In additional examples, hybrid proteins are formed from an extracellular domain of a co-stimulatory ligand protein and an intracellular domain of a co-stimulatory protein.

In one example, an immune cell naturally expresses 4-1BB and is genetically engineered to express a hybrid co-stimulatory protein including the extracellular domain of 4-1BBL and the intracellular signaling domain of OX40. In this example, in the presence of 4-1BB, the cell would receive 4-1BB and OX40 activation signals.

In another example, an immune cell naturally expresses 4-1BB and is genetically engineered to express two hybrid co-stimulatory proteins disclosed herein, for example, a hybrid co-stimulatory protein including the extracellular domain of 4-1BBL and the intracellular signaling domain of OX40 and a hybrid co-stimulatory protein including the extracellular domain of 4-1BBL and the intracellular signaling domain of CD40. In this example, in the presence of 4-1BB, the cell would receive 4-1BB, OX40, and CD40 activation signals.

Numerous hybrid stimulatory proteins can be generated in various combinations and expressed in cells to tailor activation signals triggered in the presence of a particular ligand. Exemplary sequences of stimulatory and co-stimulatory immune cell stimulating proteins are provided in FIG. 32A. Those of ordinary skill in the art can identify within these sequences relevant extracellular domains, intracellular domains, and transmembrane domains. FIG. 32B provides exemplary extracellular domains and FIG. 32C provides exemplary intracellular domains.

In certain examples, hybrid proteins are formed between segments of native proteins with a common feature. For example, hybrid proteins can be formed from two proteins that both naturally trimerize. In particular embodiments, hybrid proteins that naturally trimerize include: OX40, CD27, CD30, CD40, HVEM, DR3, 41BB, GITR, and LIGHT. Alternatively, hybrid proteins can be formed from two proteins that both naturally dimerize. In particular embodiments, hybrid proteins that naturally dimerize include: CD2, CD28, CD226, CRTAM (CD355), HAVCR1 (TIM1), ICOS, SLAM (CD150 or SLAMF1), Ly9 (SLAMF3), CD84 (SLAMF5), SLAMF7 (CRACC or CD319), and BLAME (SLAMF8).

Particular examples of hybrid proteins include a: 4-1BBL-OX40 hybrid (e.g., OX40 ICD—4-1BBL TM—4-1BBL ECD); mu41BBL-OX40 hybrid (e.g., Met—OX40 ICD—mu41BBL TM—mu41BBL ECD); 41BBL-CD27 hybrid (e.g., Met—CD27 ICD—4-1BBL TM—4-1BBL ECD); mu41BBL-CD27 hybrid (e.g., Met—CD27 ICD—mu41BBL TM—mu41BBL ECD); 41BBL-CD30 hybrid (e.g., Met—CD30 ICD—4-1BBL TM—4-1BBL ECD); mu41BBL-CD30 hybrid (e.g., Met—CD30 ICD—mu41BBL TM—mu41BBL ECD); 41BBL-CD40 hybrid (e.g., Met—CD40 ICD—4-1BBL TM—4-1BBL ECD); mu41BBL-CD40 hybrid (e.g., Met—CD40 ICD—mu41BBL TM—mu41BBL ECD); 41BBL-HVEM hybrid (e.g., Met—HVEM ICD—4-1BBL TM—4-1BBL ECD); mu41BBL-HVEM hybrid (e.g., Met—HVEM ICD—mu41BBL TM—mu41BBL ECD); 41BBL-DR3 hybrid (e.g., Met—DR3 ICD—4-1BBL TM—4-1BBL ECD); mu41BBL-DR3 hybrid (e.g., Met—DR3 ICD—mu41BBL TM—mu41BBL ECD); 41BBL-41BB hybrid (e.g., Met—4-1BB ICD—4-1BBL TM—4-1BBL ECD); mu41BBL-41BB hybrid (e.g., Met—41BB ICD—mu41BBL TM—mu41BBL ECD); OX40L-OX40 hybrid (e.g., Met—OX40 ICD—OX40L TM—OX40L ECD); muOX40L-OX40 hybrid (e.g., Met—OX40 ICD—muOX40L TM—muOX40L ECD); OX40L-CD27 hybrid (e.g., Met—CD27 ICD—OX40L TM—OX40L ECD); muOX40L-CD27 hybrid (e.g., Met—CD27 ICD—muOX40L TM—muOX40L ECD); OX40L-CD30 hybrid (e.g., Met—CD30 ICD—OX40L TM—OX40L ECD); muOX40L-CD30 hybrid (e.g., Signal Peptide—CD30 ICD—muOX40L TM—muOX40L ECD); OX40L-CD40 hybrid (e.g., Met—CD40 ICD—OX40L TM—OX40L ECD); muOX40L-CD40 hybrid (e.g., Signal Peptide—CD40 ICD—muOX40L TM—muOX40L ECD); OX40L-HVEM hybrid (e.g., Met—HVEM ICD—OX40L TM—OX40L ECD); muOX40L-HVEM hybrid (e.g., Signal Peptide—HVEM ICD—muOX40L TM—muOX40L ECD); OX40L-DR3 hybrid (e.g., Met—DR3 ICD—OX40L TM—OX40L ECD); muOX40L-DR3 hybrid (e.g., Signal Peptide—DR3 ICD—muOX40L TM—muOX40L ECD); OX40L-41BB hybrid (e.g., Met—4-1BB ICD—OX40L TM—OX40L ECD); muOX40L-41BB hybrid (e.g., Signal Peptide—41BB ICD—muOX40L TM—muOX40L ECD); CD40L-OX40 hybrid (e.g., Met—OX40 ICD—CD40L TM—CD40L ECD); muCD40L-OX40 hybrid (e.g., Signal Peptide—OX40 ICD—muCD40L TM—muCD40L ECD); CD40L-CD27 hybrid (e.g., Met—CD27 ICD—CD40L TM—CD40L ECD); muCD40L-CD27 hybrid (e.g., Signal Peptide—CD27 ICD—muCD40L TM—muCD40L ECD); CD40L-CD30 hybrid (e.g., Met—CD30 ICD—CD40L TM—CD40L ECD); muCD40L-CD30 hybrid (e.g., Signal Peptide—CD30 ICD—muCD40L TM—muCD40L ECD); CD40L-CD40 hybrid (e.g., Met—CD40 ICD—CD40L TM—CD40L ECD); muCD40L-CD40 hybrid (e.g., Signal Peptide—CD40 ICD—muCD40L TM—muCD40L ECD); CD40L-HVEM hybrid (e.g., Met—HVEM ICD—CD40L TM—CD40L ECD); muCD40L-HVEM hybrid (e.g., Signal Peptide—HVEM ICD—muCD40L TM—muCD40L ECD); CD40L-DR3 hybrid (e.g., Met—DR3 ICD—CD40L TM—CD40L ECD); and ; muCD40L-DR3 hybrid (e.g., Signal Peptide—DR3 ICD—muCD40L TM—muCD40L ECD); CD40L-41BB hybrid (e.g., Met—4-1BB ICD—CD40L TM—CD40L ECD); muCD40L-41BB hybrid (e.g., Signal Peptide—41BB ICD—muCD40L TM—muCD40L ECD); huFLT3L-OX40 hybrid (e.g., Signal Peptide—huFLT3L ECD-huFLT3L TM—OX40 ICD); huFLT3L-CD27 hybrid (e.g., Signal Peptide—huFLT3L ECD-huFLT3L TM—CD27 ICD); huFLT3L-CD30 hybrid (e.g., Signal Peptide—huFLT3L ECD-huFLT3L TM—CD30 ICD); huFLT3L-CD40 hybrid (e.g., Signal Peptide—huFLT3L ECD-huFLT3L TM—CD40 ICD); huFLT3L-HVEM hybrid (e.g., Signal Peptide—huFLT3L ECD-huFLT3L TM—HVEM ICD); huFLT3L-DR3 hybrid (e.g., Signal Peptide—huFLT3L ECD-huFLT3L TM—DR3 ICD); huFLT3L-41BB hybrid (e.g., Signal Peptide—huFLT3L ECD-huFLT3L TM—41BB ICD); huLIGHT-OX40 hybrid (e.g., Met—OX40 ICD—huLIGHT TM—huLIGHT ECD); huLIGHT-CD27 hybrid (e.g., Met—CD27 ICD—huLIGHT TM—huLIGHT ECD); huLIGHT-CD30 hybrid (e.g., Met—CD30 ICD—huLIGHT TM—huLIGHT ECD); huLIGHT-CD40 hybrid (e.g., Met—CD40 ICD—huLIGHT TM—huLIGHT ECD); huLIGHT-HVEM hybrid (e.g., Met—HVEM ICD—huLIGHT TM—huLIGHT ECD); huLIGHT-DR3 hybrid (e.g., Met—DR3 ICD—huLIGHT TM—huLIGHT ECD); and; huLIGHT-41BB hybrid (e.g., Met—41BB ICD—huLIGHT TM—huLIGHT ECD)). See FIG. 32F for even more particular examples.

The disclosed hybrid protein approach differs from existing technology by providing stimulatory domains that allow for multiple signaling combinations, providing a greater boost to stimulation than one domain. Hybrid proteins disclosed herein provide a novel platform to provide enhanced signals, for example, to simultaneously improve the function of adoptive cell therapies and engage the diversity of endogenous immune cells to provide an orchestrated immune response against cancerous or infected cells. Similar to checkpoint blockade or co-stimulatory agonist drugs, hybrid proteins disclosed herein will also “release the brakes” from endogenous immune cells, however important differences are that this approach avoids the toxicities of drug therapies (e.g. liver toxicity) and issues with drug delivery to the tumor.

4-1BB is a membrane receptor protein of the Tumor Necrosis Factor receptor superfamily (TNFRSF) with OX40, CD40, CD27, TNFR-I, TNFR-II, Fas, CD30, and DR3 (see, e.g., Alderson et al., Eur. J. Immunol. 24:2219 (1994)). 4-1BB is also referred to as CD137 and TNFRSF Member 9 (TNFRSF9). 4-1BB is expressed on the surface of activated T cells as a type of accessory protein (Kwon et al., Proc. Natl. Acad. Sci. USA 86:1963 (1989); Pollok et al., J. Immunol. 151:771 (1993)).

4-1BB has a molecular weight of 55 kDa and forms a trimer upon binding to a high-affinity ligand (4-1BB, also termed CD137L) expressed on several APCs such as macrophages and activated B cells (Pollok et al., J. Immunol. 150:771 (1993) Schwarz et al., Blood 85:1043 (1995)) as well as myeloid progenitor cells, and hematopoietic stem cells. The interaction of 4-1BB and its ligand provides a co-stimulatory signal leading to T cell activation and growth (Goodwin et al., Eur. J. Immunol. 23:2631 (1993); Alderson et al., Eur. J. Immunol. 24:2219 (1994); Hurtado et al., J. Immunol. 155:3360 (1995); Pollock et al., Eur. J. Immunol. 25:488 (1995); DeBenedette et al., J. Exp. Med. 181:985 (1995)). Signaling via 4-1BB prompts cytokine induction, prevention of activation-induced cells death (AICD), upregulation of CTL activity, and increased survival. With the administration of 4-1BB in vivo, studies show a robust activation of CD8+ T cells and tumor suppression (Vinay, 2014, BMB Rep. 47 (3): 122-129).

OX40, also referred to as CD134, TNFRSF member 4 (TNFRSF4), ACT35 and TXGP1L, is a 50 kDa glycoprotein. The ligand for OX40, OX40L (also referred to as CD252), has been reported to be expressed on endothelial cells and activated APCs including macrophages, dendritic cells, B cells and natural killer cells. Binding between CD40 on APCs increases OX40L expression. Expression of OX40 on T cells can be induced following signaling via the T cell antigen receptor. For example, OX40 is expressed on recently activated T cells at the site of inflammation. CD4 and CD8 T cells can upregulate OX40 under inflammatory conditions. Costimulatory signals from OX40 promote T cell division, survival, and suppress the differentiation and activity of Treg T cells (Croft Immunol Rev 2009).

CD40, also referred to as TNFRSF member 5 (TNFRSFS), or CD40 ligand receptor is a costimulatory protein found on APCs and is required for activation. CD40 contains 277 amino acids of which 20 amino acids at the N terminus represent the signal sequence. A transmembrane domain is located at resides 194-215 and the cytoplasmic domain is located at residues 216-277. The nucleotide sequence of CD40 (1177 bp) is available in public databases (see Genbank accession no. NM—001250). CD40 and various isoforms are described by Tone et al. Proc. Natl. Acad. Sci. U.S.A. 98 (4), 1751-1756 (2001). CD40 is expressed by monocytes and B cells binds to CD4O-L (a.k.a. CD40 ligand or CD153) expressed by activated T cells.

CD27 is a TNFRSF member that is a transmembrane protein. It is expressed on the majority of CD4+ and CD8+ resting T cells. The ligand for CD27 is CD70 and their interaction enhances T cell activation with regards to proliferation. Improved signaling of CD27 is shown with hexamerization (Thieman et al., Front. Oncol. 8, 2018).

CD30 is a TNFRSF member that is often expressed in hematopoietic malignancies such as large cell lymphoma and Hodgkin lymphoma. The CD30 ligand, also referred to as CD30L, TNFSF8, or CD153, is a membrane-bound cytokine. CD30 signaling controls T-cell survival, regulates peripheral T-cell responses, and downregulates cytolytic capacity (Wu, et al., Immune Biology of Allogeneic Hematopoietic Stem Cell Transplantation (Second Edition), 2019).

FMS like tyrosine kinase 3 (FLT3) is also referred to as CD135. FLT3 is a cytokine receptor which belongs to the receptor tyrosine kinase class III. Its ligand, FLT3L stimulates the proliferation of stem and progenitor cells upon binding with FLT3.

LIGHT (also known as tumor necrosis superfamily member 14, CD258, and HVEML) is a secreted protein of the TNF superfamily. Upon binding its ligand, herpesvirus entry mediator (HVEM), LIGHT interacts with two receptors, lyphotoxin13 receptor (LTβR) and herpesvirus entry mediator (HVEM) to enhance T cell proliferation and cytokine production.

Herpesvirus entry mediator (HVEM) is the specific ligand for B- and T-lymphocyte attenuator (BTLA). BTLA is an immune-regulatory receptor that is expressed on B- and T-, and all mature lymphocytes. BTLA, also referred to as CD272, is in the CD38 family along with PD1 and CTLA-4 while HVEM belongs to the TNFR family. The interaction of HVEM and BTLA plays an important role in immune tolerance and immune response (Yu et al., 2019, Front. Immunol. ht tps://doi.org/10.3389/fimmu.2019.00617).

Death receptor 3 (DR3) is also referred to as TRAMP, LARD, WSL-1, and TNFRSF member 25 (TNRFSF25). DR3 is a death-domain-containing tumor necrosis factor family receptor expressed on T cells. Its ligand, TL1A (also referred to as TNFSF15 or VEGI), costimulates T cells to produce a wide variety of cytokines and can promote expansion of activated and regulatory T cells. DR3 costimulates T cell activation and is unique because it signals through an intracytoplasmic death domain and the adapter protein TRADD (Meylan, et al., 2011. Immunol Rev. 244(1): 10.1111).

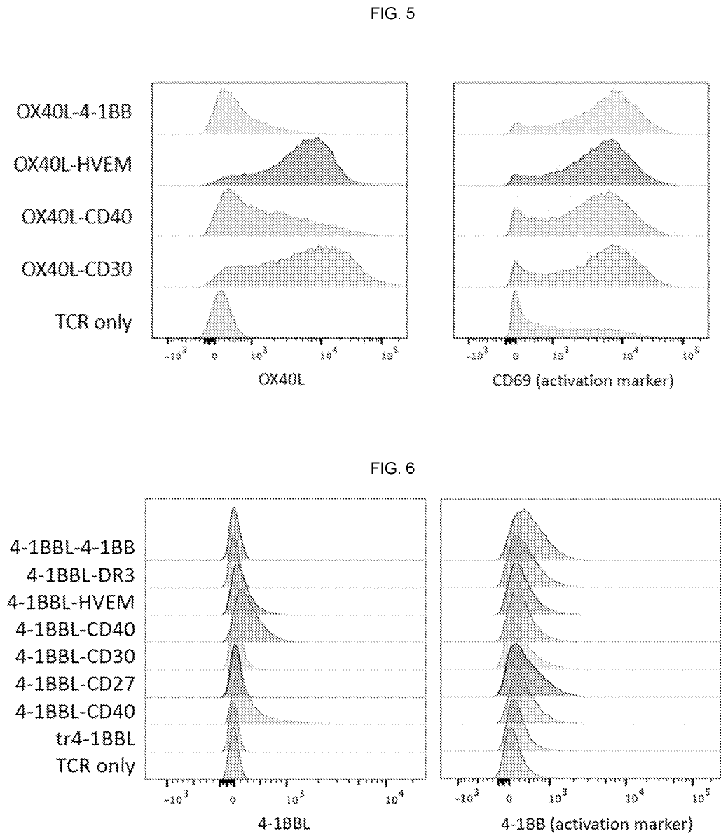

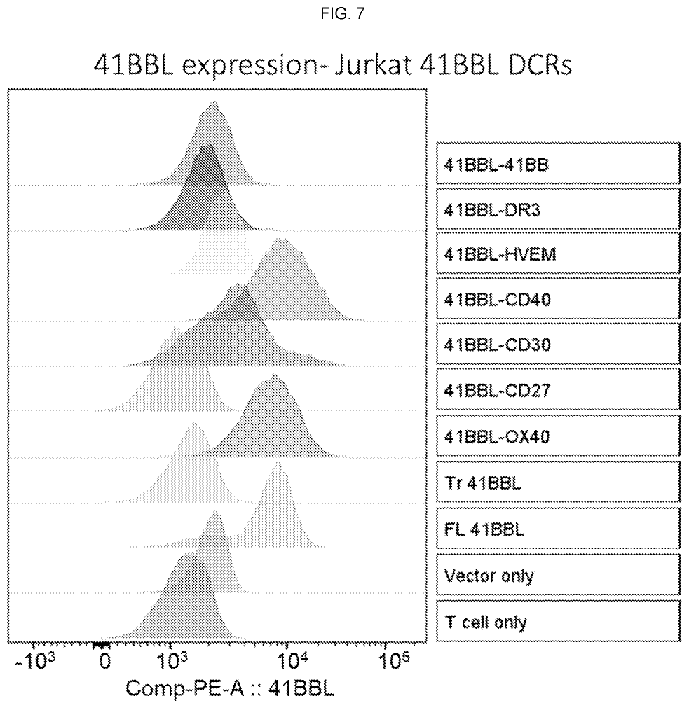

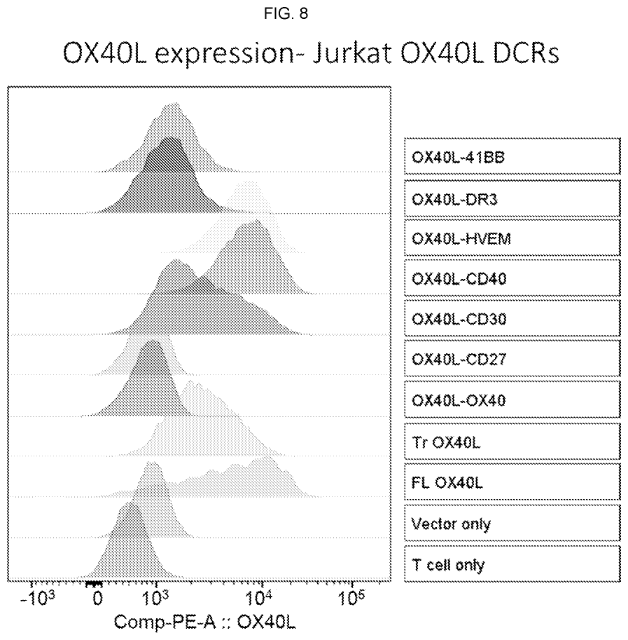

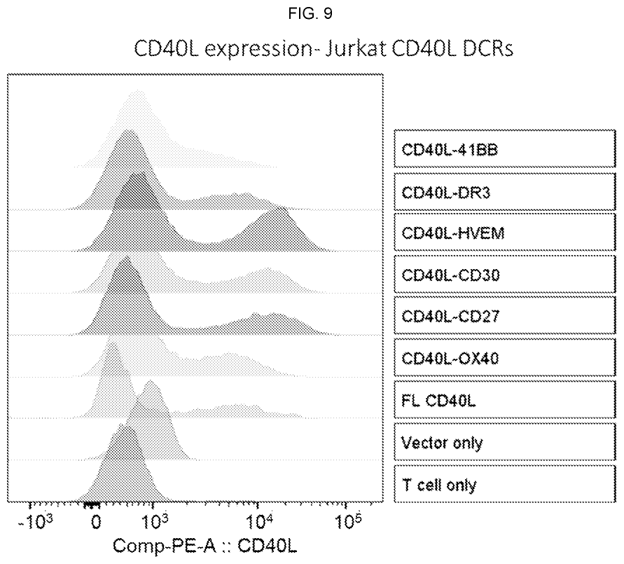

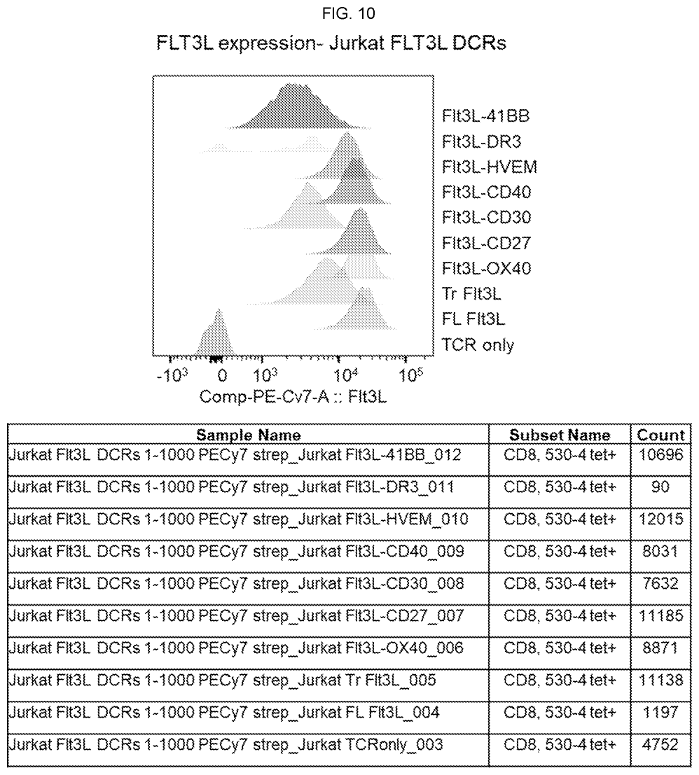

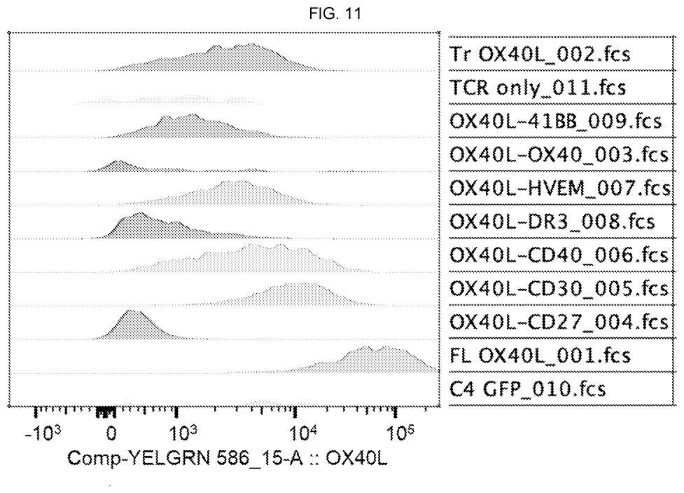

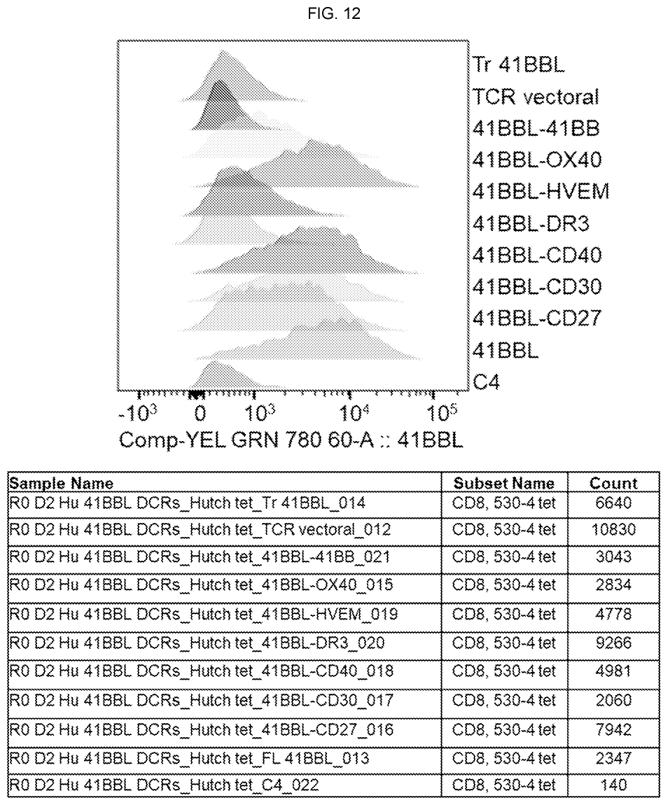

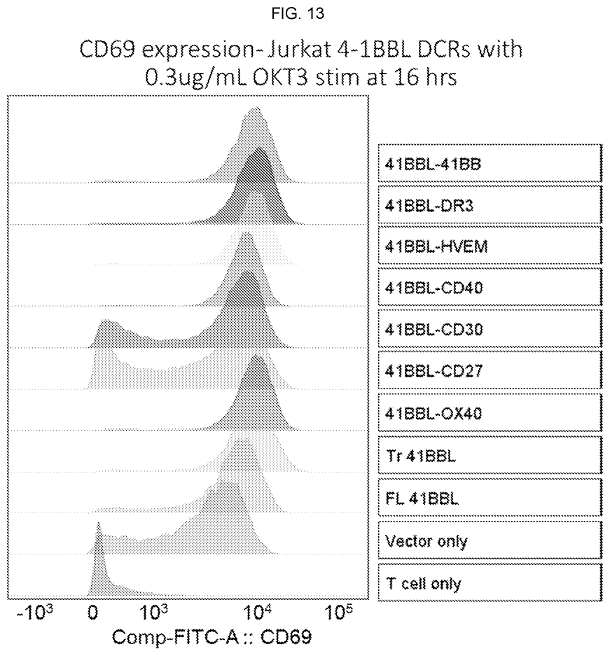

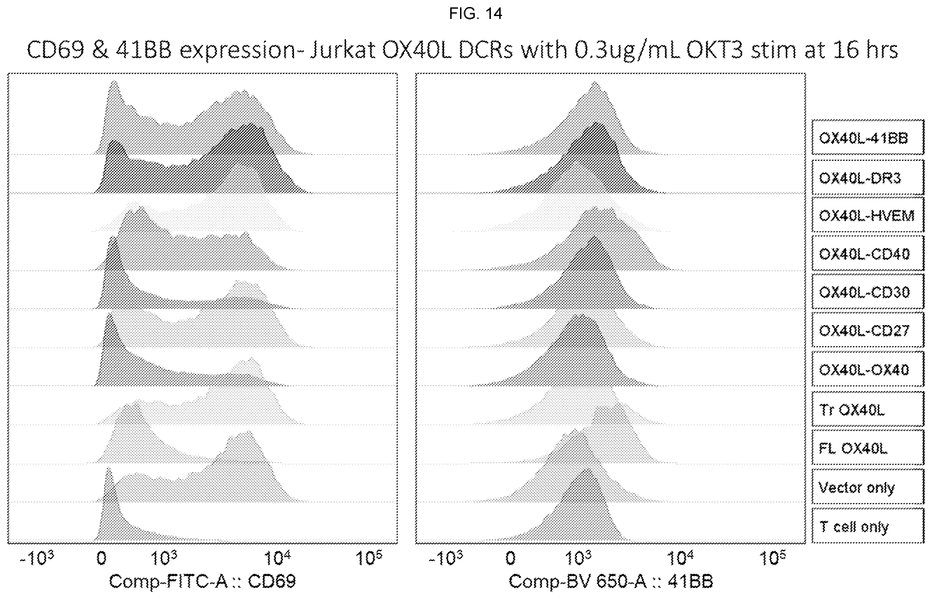

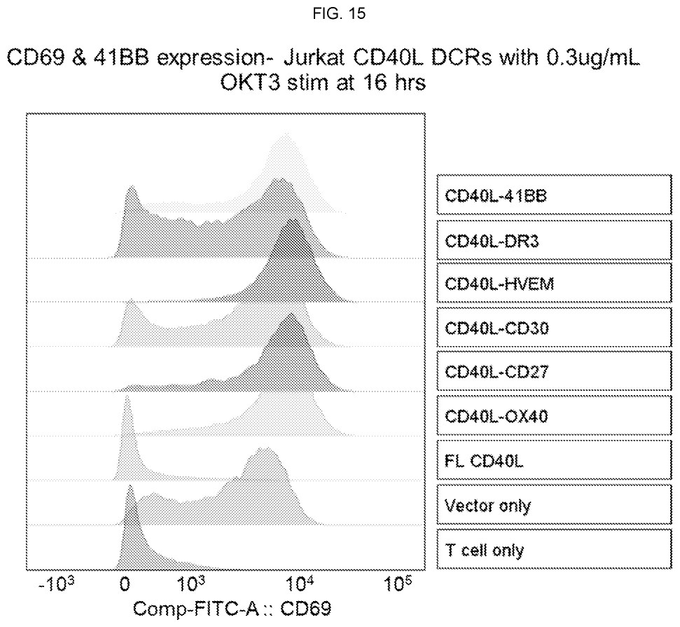

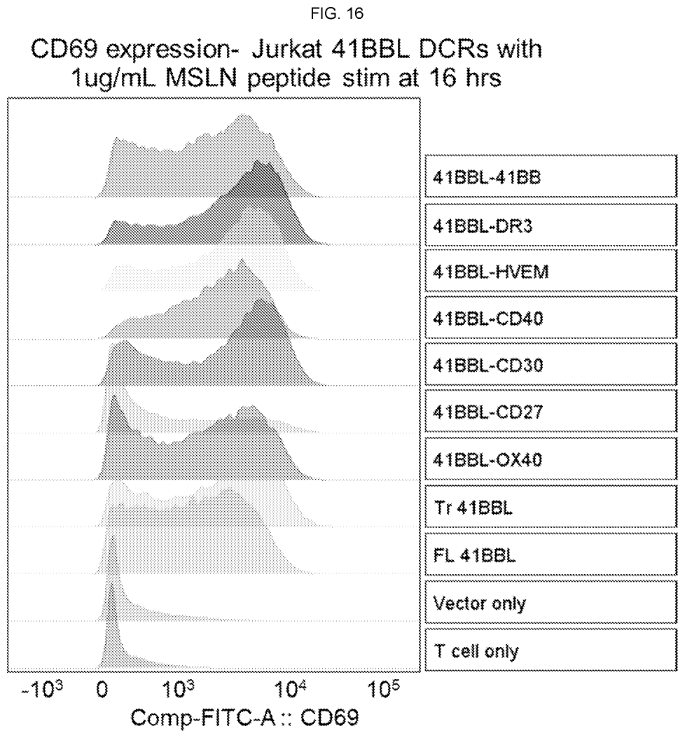

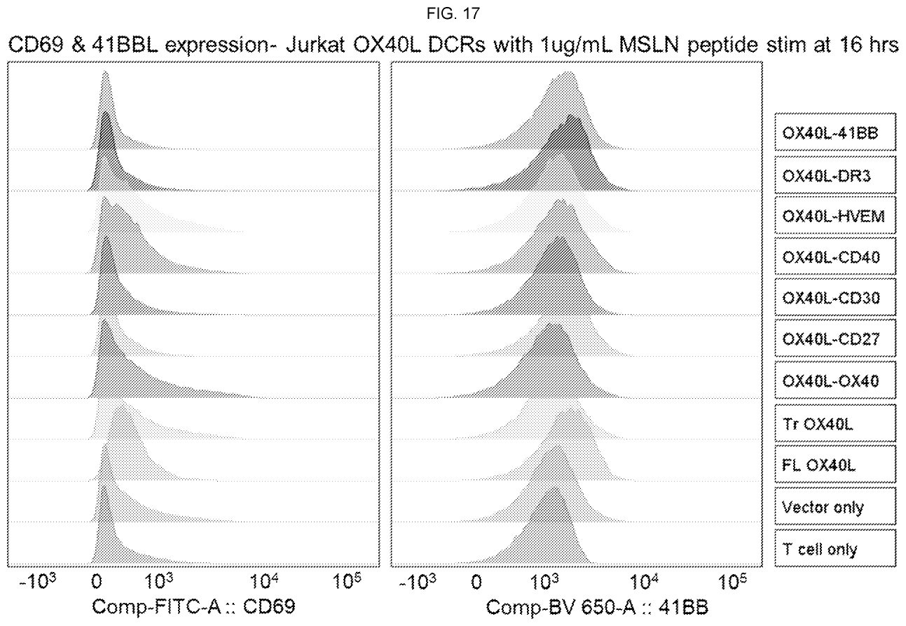

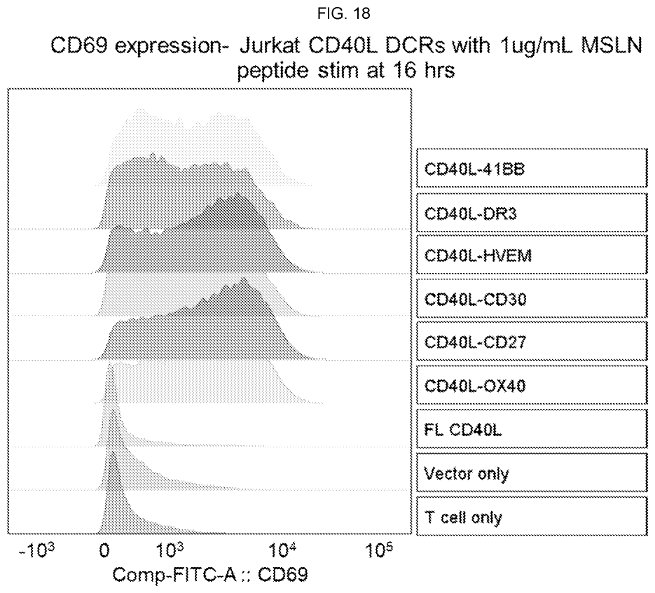

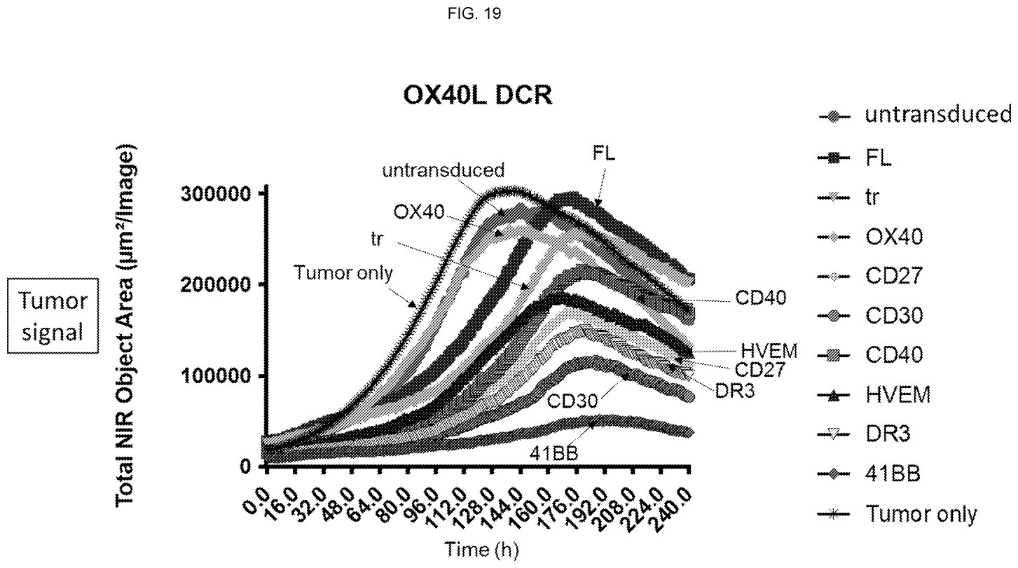

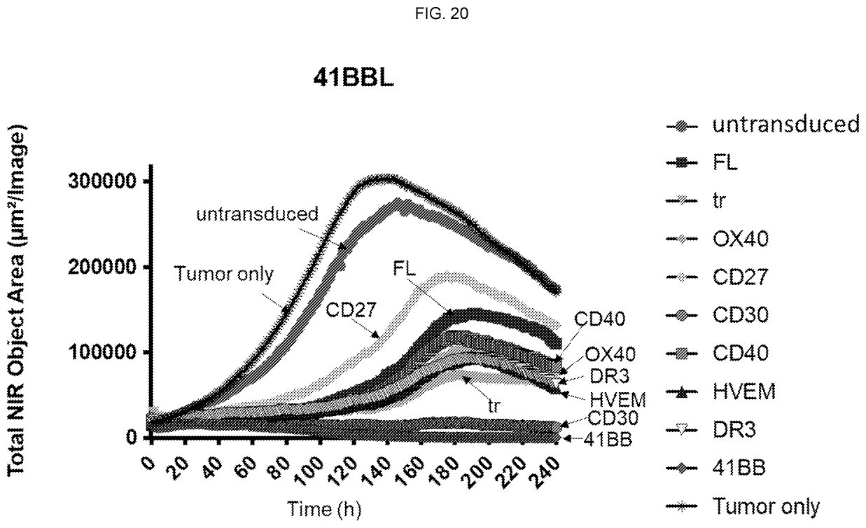

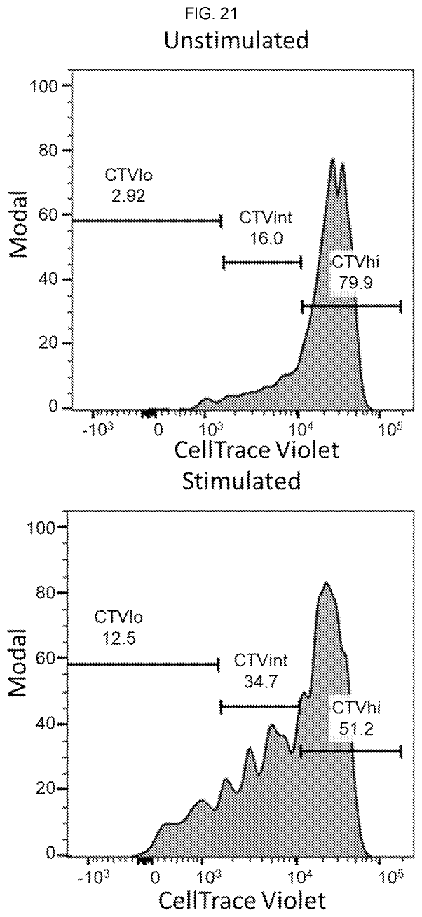

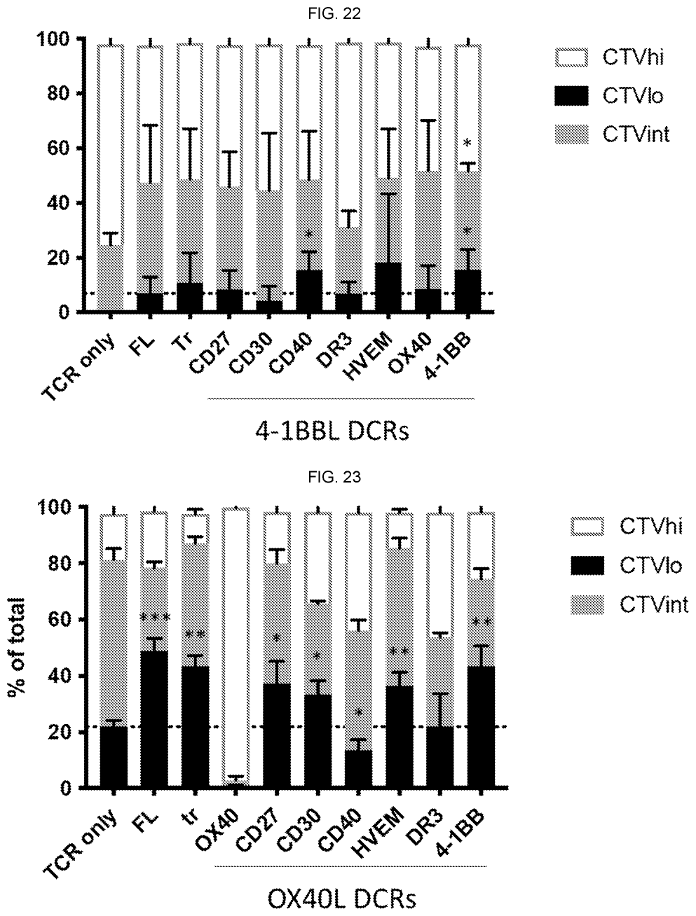

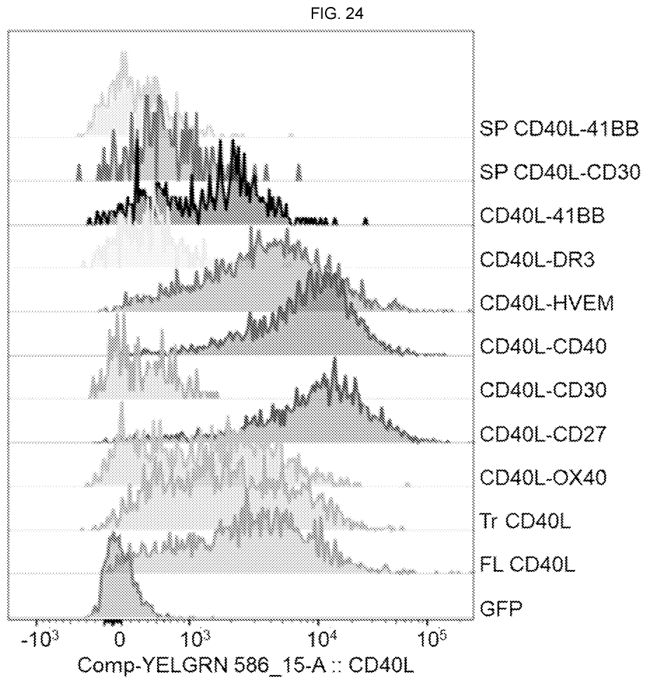

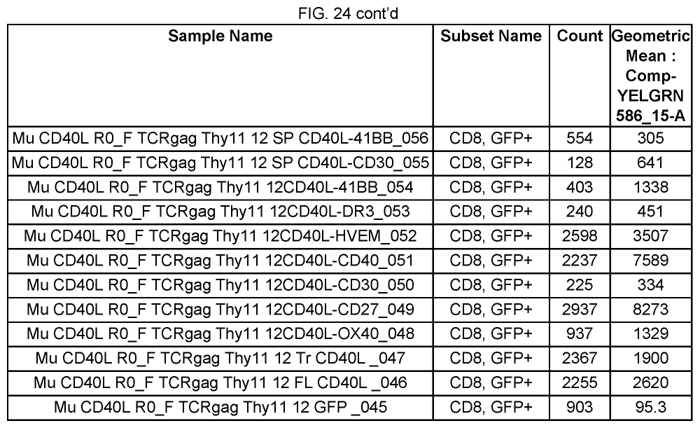

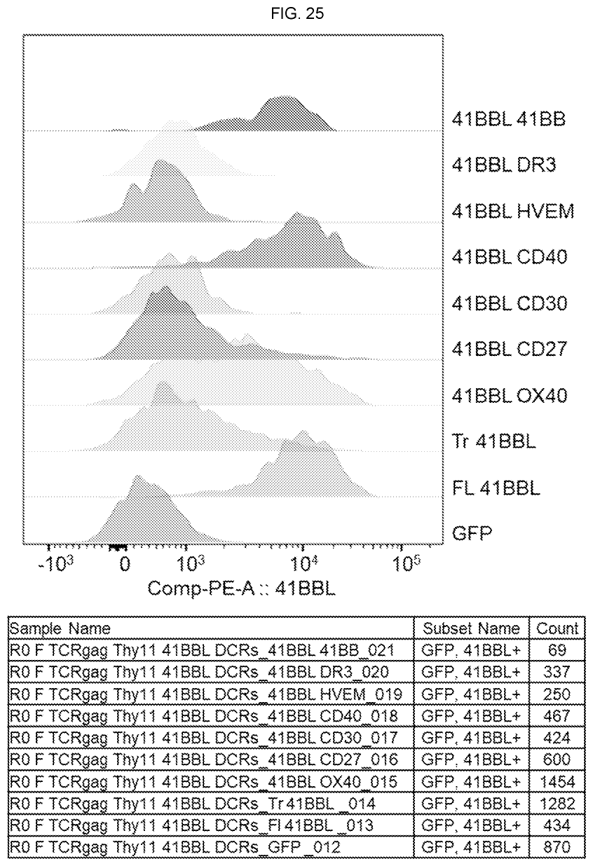

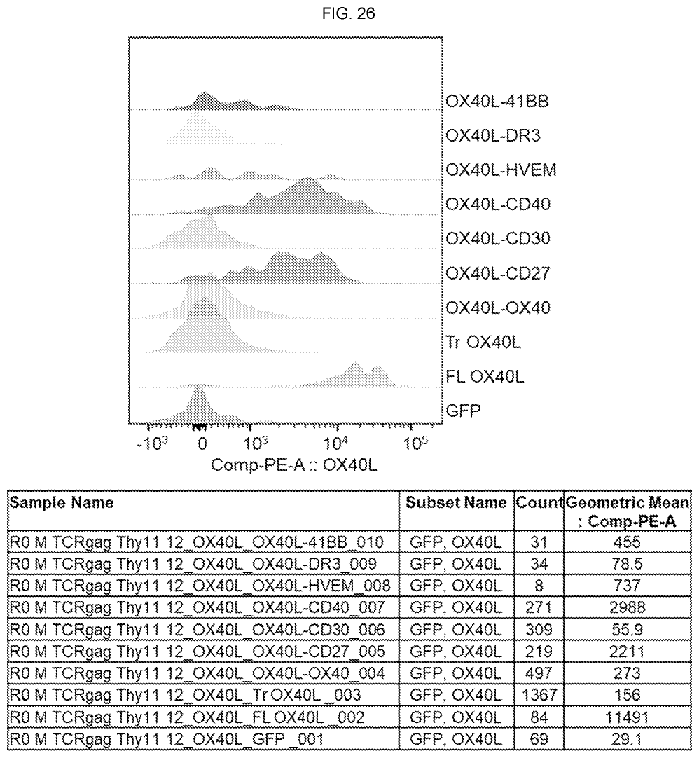

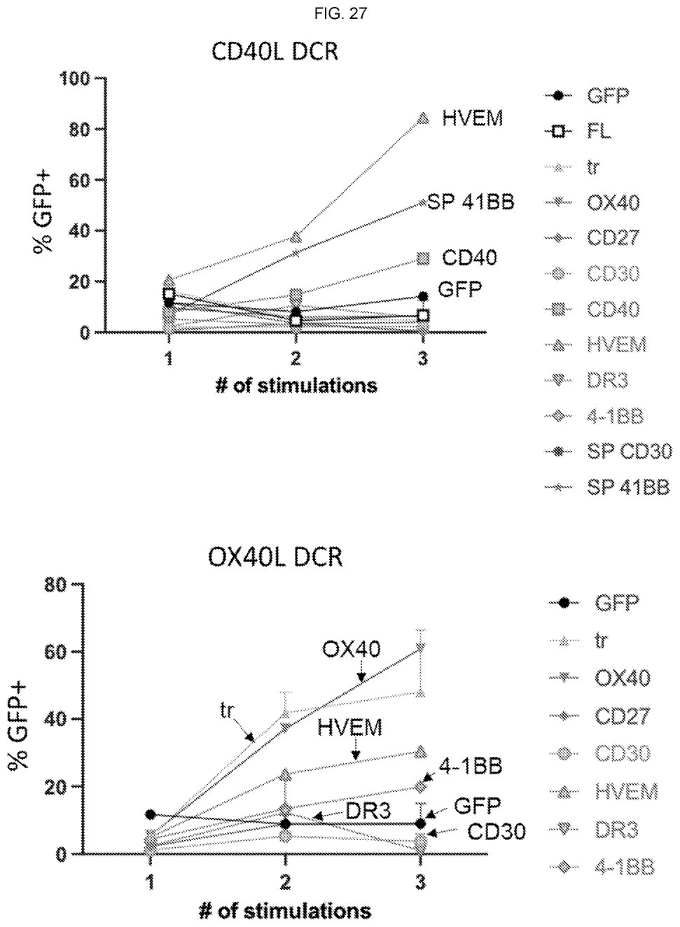

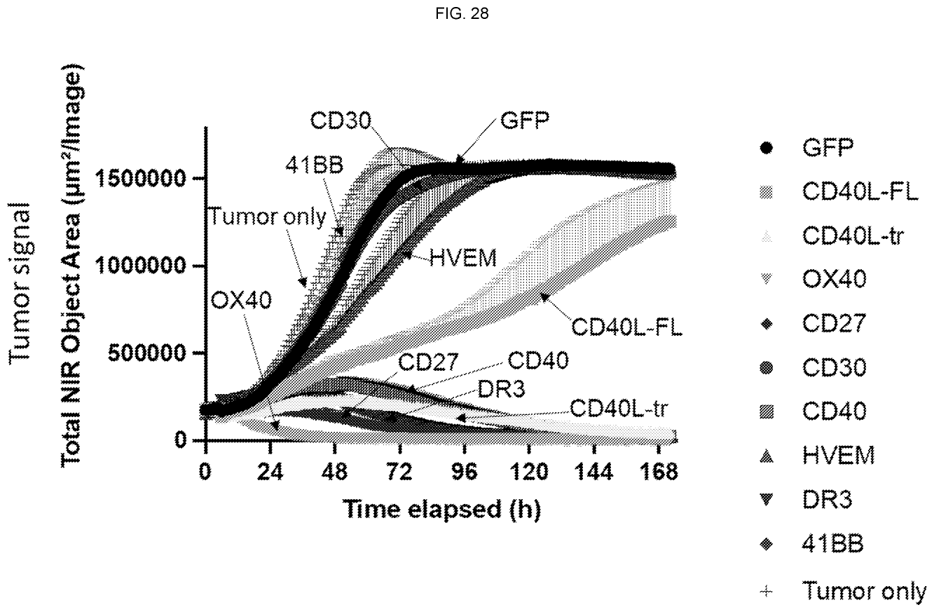

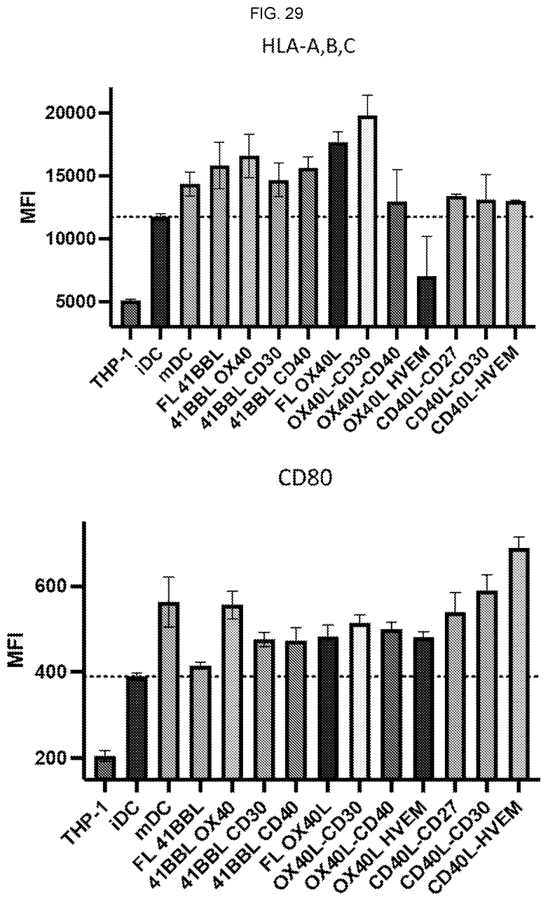

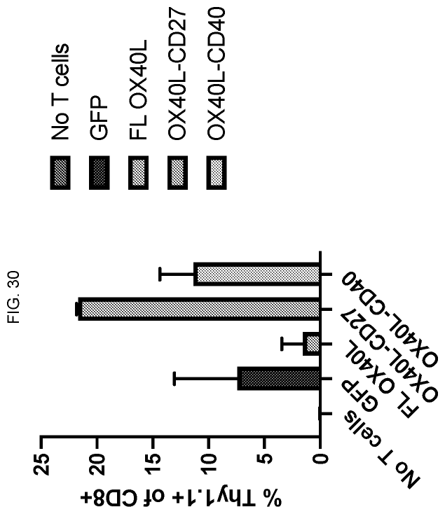

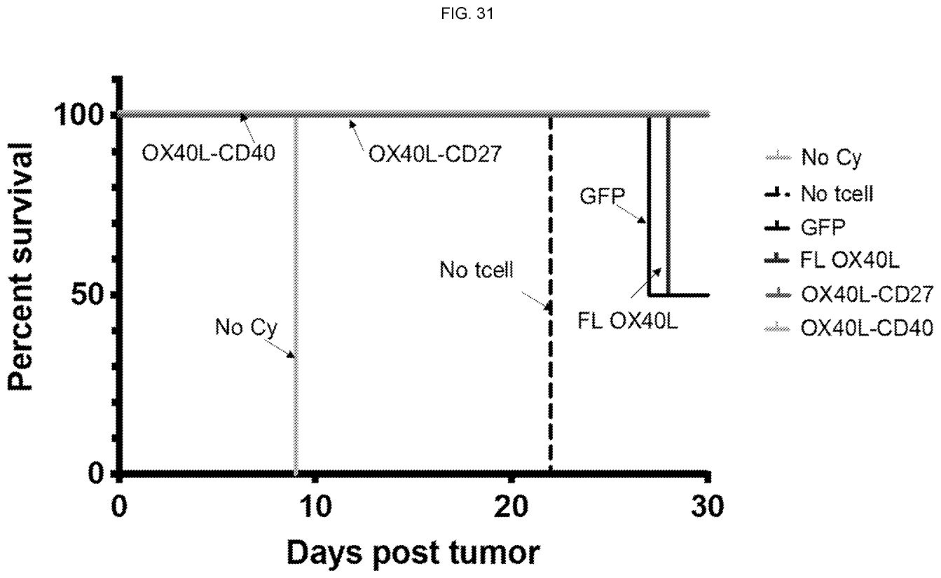

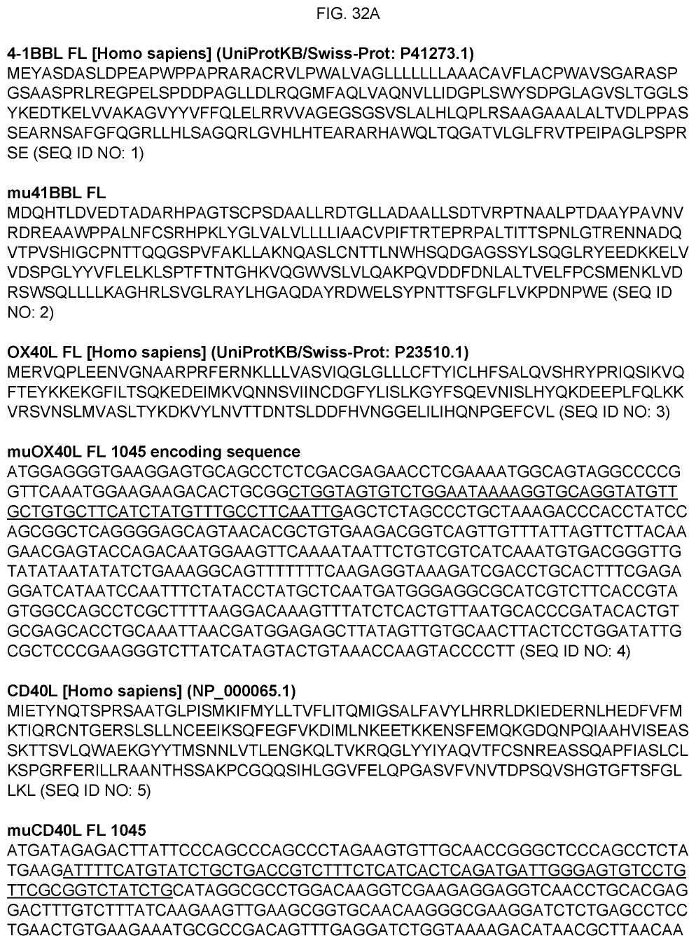

While FIGS. 32B, 32C, 32E, and 32F provide exemplary extracellular and intracellular domains of stimulatory and co-stimulatory proteins, one of ordinary skill in the art will recognize that the disclosure also encompasses functional variants and functional fragments of these protein segments. Functional variants include one or more residue additions or substitutions that do not substantially impact the physiological effects of the protein. Functional fragments include one or more deletions or truncations that do not substantially impact the physiological effects of the protein. In hybrid proteins disclosed herein the extracellular domain retains ability to bind it's natural binding partner and the intracellular domain includes at least that portion of an intracellular domain that is sufficient to transduce an activation signal, for example by associating with or activating a cytoplasmic signaling protein. A lack of substantial impact can be confirmed by observing experimentally comparable results in a binding assay and/or in a measure of immune cell activation. Measures of immune cell activation include, for example, cytokine release, proliferation and expansion, tumor cell lysis, and changed marker profile expression (e.g., CD69, 4-1BB upregulation in activated T cells, for example as shown in FIG. 5).

Immune cell proteins that can be used to create truncated decoy proteins described herein include 4-1BBL (CD137L), OX40L (CD134L), CD40L, CD30L, CD27L, DR3L, LIGHT, SLAMF1L, ICOSL, GITRL, CD80, CD86, CD79AL, CD79BL, CARD11 L, DAP10L, DAP12L, FcRαL, FcRβL, FcRγL, FLT3L, FynL, LckL, LATL, LIGHT, LRPL, NKG2DL, NOTCH1L, NOTCH2L, NOTCH3L, NOTCH4L, ROR2L, RykL, Slp76L, pTαL, TIM1L, TRIML, Zap70L, and PTCH2L.

In these truncations, a sufficient number of intracellular residues are removed so that intracellular signals are no longer transmitted following binding. The truncations can include, for example, 5 amino acid residue truncations, 6 amino acid residue truncations, 7 amino acid residue truncations, 8 amino acid residue truncations, 9 amino acid residue truncations, 10 amino acid residue truncations, 11 amino acid residue truncations, 12 amino acid residue truncations, 13 amino acid residue truncations, 14 amino acid residue truncations, 15 amino acid residue truncations, 16 amino acid residue truncations, 17 amino acid residue truncations, 18 amino acid residue truncations, 19 amino acid residue truncations, 20 amino acid residue truncations, 21 amino acid residue truncations, 22 amino acid residue truncations, 23 amino acid residue truncations, 24 amino acid residue truncations, or 25 amino acid residue truncations. The truncations can be consecutive amino acid truncations or intermittent amino acid truncations. In particular embodiments, entire intracellular domains are removed. Other truncations can leave 1, 2, 3, 4, 5, 6, 7, 8, 9, or 10 membrane-proximal intracellular residues of an intracellular signaling domain, so long as the remaining residues do not transmit intracellular signals upon ligand binding.

Within hybrid and truncated proteins disclosed herein, transmembrane domains can be any transmembrane domain that has a three-dimensional structure that is thermodynamically stable in a cell membrane. Transmembrane domains generally ranges in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. When the source is natural, the domain may be derived from any membrane-bound or transmembrane protein. In particular embodiments, a transmembrane domain may include at least the transmembrane region(s) of: the α, β, or ζ chain of the T-cell receptor; CD28; CD27; CD3ε; CD45; CD4; CDS; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; OX40L; CD2; LFA-1; ICOS; ICOSL; 4-1BB; 4-1BBL; GITR; GITRL; CD40; CD40L; CD30; CD30L; FLT3; FLT3L; Fyn; FynL; Lck; LckL; LAT; LATL; LRP; LRPL; LIGHT; DR3; DR3L; CD27; CD27L; CD25; CD28; CD80; CD86,; CD79a; CD79aL; CD79b; CD79bL; CD84 (SLAMFS); DAP10; DAP10L; DAP12; DAP12L; BAFFR; HVEM; SLAMF7; NKp80; NKp44; NKp30; NKp46; NOTCH1; NOTCH1L; NOTCH2; NOTCH2L; NOTCH3; NOTCH3L; NOTCH4; NOTCH4L; ROR2; ROR2L; Ryk; RykL; Slp76; Slp76L; pTα; pTαL; TCRα; TCR3β; HAVCR1 (TIM1); TIM1L; TRIM; TRIML; Zap70; Zap70L; PTCH2; PTCH2L; CD122; CD132; CD226; CD160; CD19; CARD11; CARD11L; CRTAM (CD355); IL2Rβ; IL2Rβ; IL7Ra; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DNAM1; SLAMF1 (CD150 or SLAM); SLAMF1L; SLAMF4; CD84; CD96; CEACAM1; CRT AM; Ly9 (SLAMF3); PSGL1; CD100; SLAMF6 (NTB-A, Lyl08); BLAME (SLAMF8); SELPLG; LTBR; PAG/Cbp; NKG2D; NKG2DL; NKG2C; FcRα; FcRαL; FcRβ; FcRβL; FcRγ; FcRγL; CD3ε; CD3δ; or CD3ζ.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. Particular exemplary transmembrane domains are provided in FIG. 32D.

In preferred embodiments, the hybrid and truncated proteins disclosed herein utilize only segments of naturally occurring proteins to minimize any potential immune response when expressed in vivo. While these embodiments are preferred, in some instances junction amino acids may be present between segments of the hybrid and truncated proteins. Junction amino acids refer to short amino acid sequences, for example, 20 amino acids or less. Exemplary glycine-serine junction amino acids include GGGGSGGGGS (SEQ ID NO: 215), GGSGGSGGS (SEQ ID NO: 216), and GGGGS (SEQ ID NO: 217). In particular embodiments, a glycine-serine (GS) doublet can be used as a suitable junction amino acid linker. In particular embodiments, a single amino acid, e.g., an alanine, a glycine, can be used as a suitable junction amino acid.

In certain embodiments, the use of spacer regions and/or any cancer or viral antigen binding domain are expressly excluded from the hybrid and truncated proteins disclosed herein. As used herein, spacer regions and cancer or viral antigen binding domains refer to these segments of proteins typically found in CAR or recombinant TCR. Examples of spacer regions that are excluded in certain embodiments include hinge regions of immunoglobulins and type II C-lectin interdomains.

In certain instances, hybrid stimulatory proteins described herein include components of a Type I protein and a Type II protein. Type I proteins include their N-terminus extracellularly when expressed while Type II proteins include their N-terminus intracellularly when expressed. Generally, costimulatory immune cell proteins (e.g., 4-1BB, OX40) are Type I proteins while costimulatory protein ligands (e.g., 4-1BBL, OX40L) are Type II proteins.

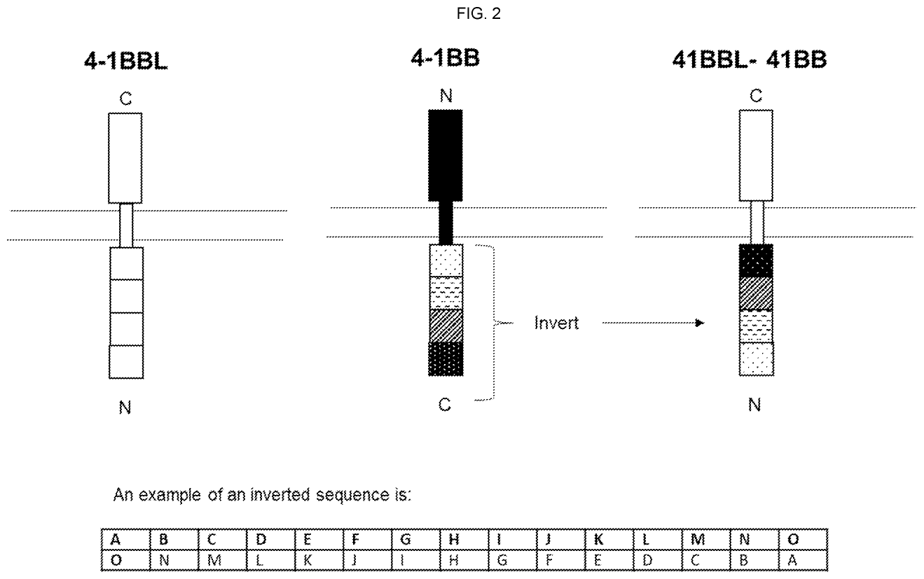

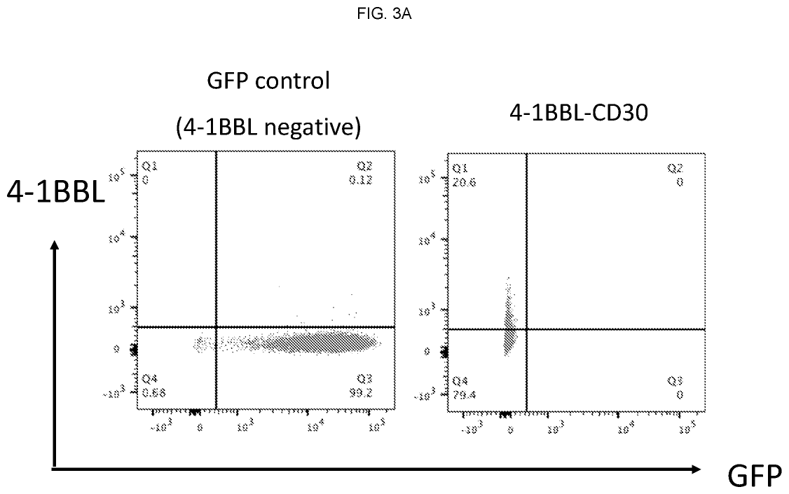

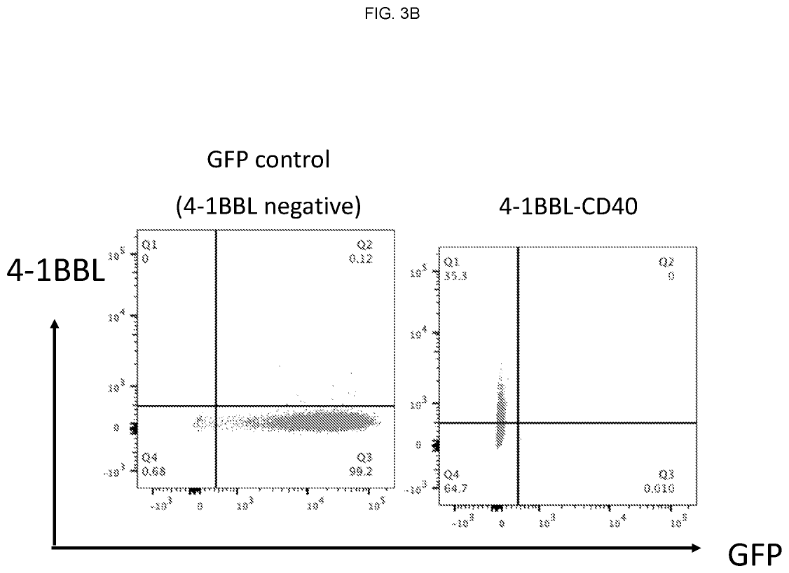

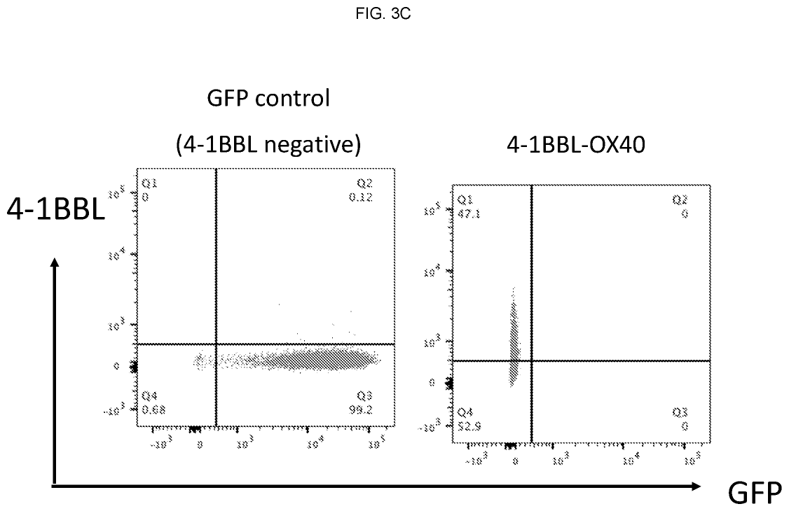

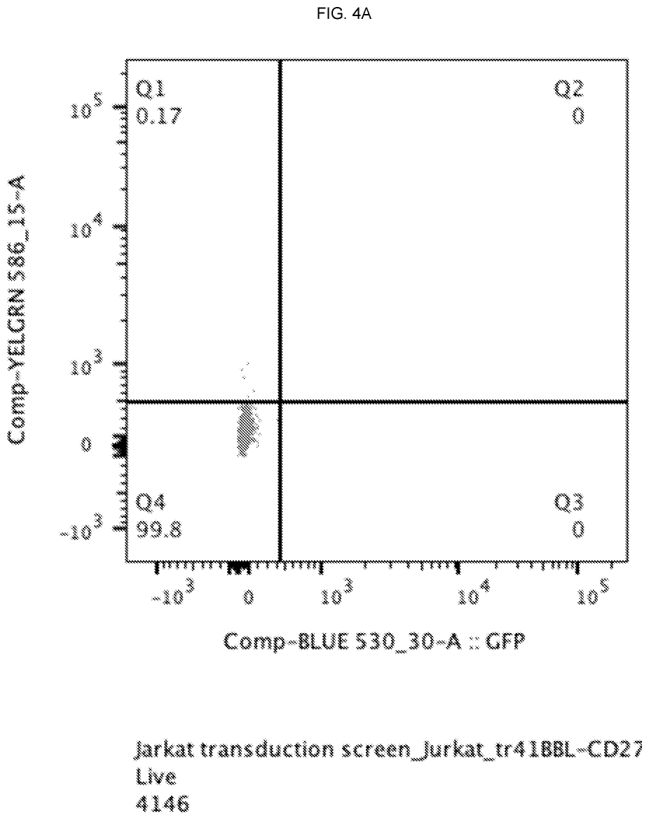

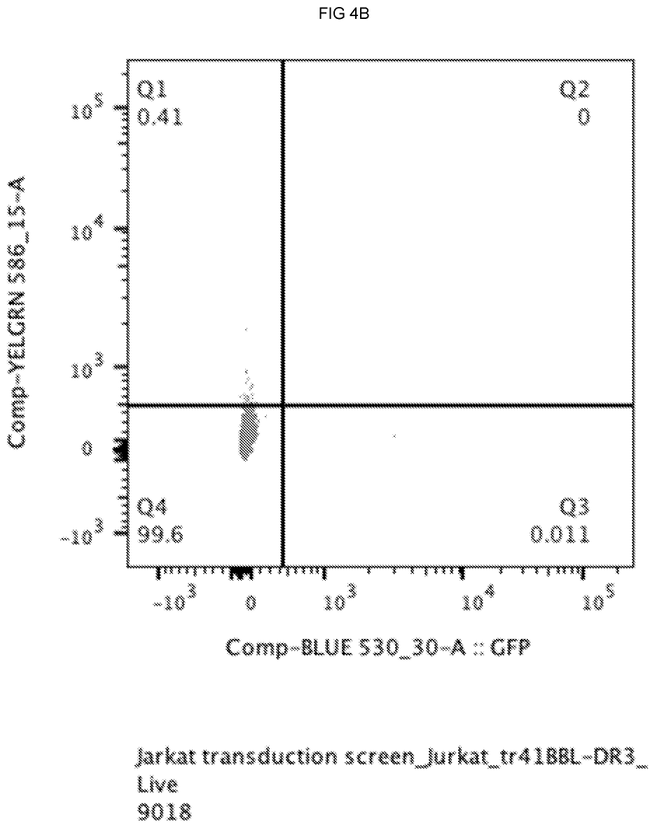

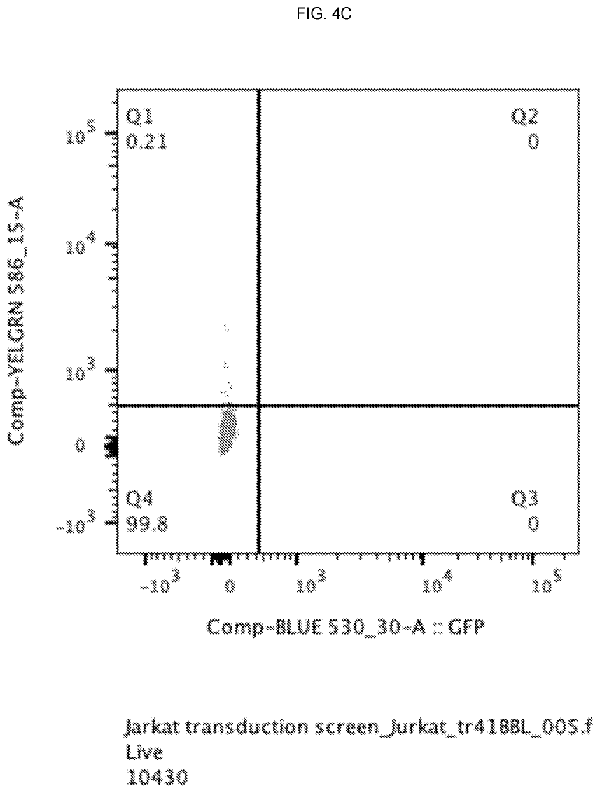

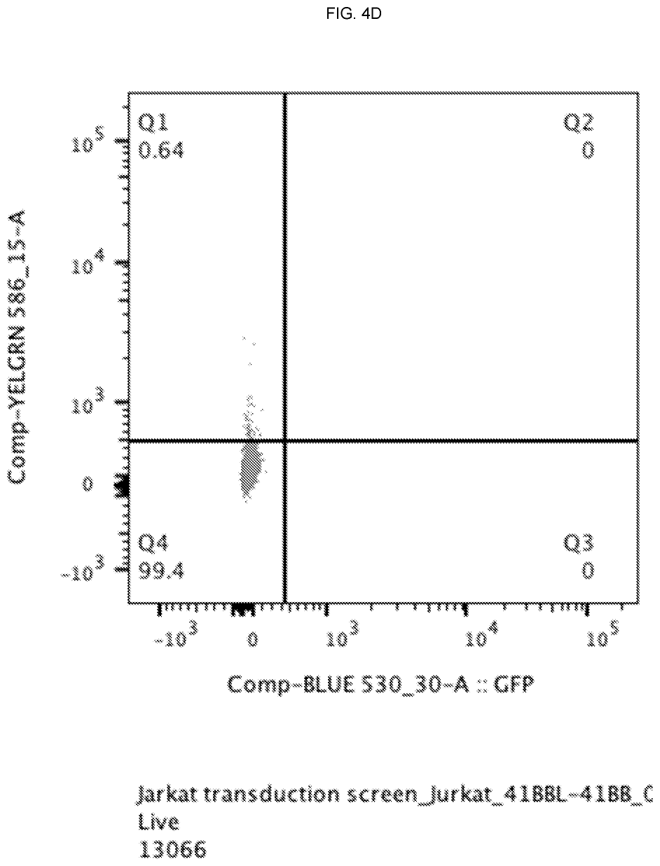

To fuse a Type I protein with a Type II protein, intracellular and/or transmembrane regions may be inverted from their native configuration (e.g., so that the intracellular tail terminus becomes the membrane proximal portion of the intracellular region; see FIG. 2). In additional embodiments, the N-terminal to C-terminal position of a stimulatory protein intracellular signaling domain is brought towards the N-terminal position. For example, from N-terminal to C-terminal expression, costimulatory immune cell proteins generally proceed from extracellular signaling domain to transmembrane domain to intracellular signaling domain. N-terminal to C-terminal expression of costimulatory immune cell protein ligands generally proceed from intracellular signaling domain to transmembrane domain to extracellular signaling domain. Certain hybrid proteins described herein include from N-terminal to C-terminal expression, an intracellular signaling domain of a costimulatory immune cell signaling domain to a transmembrane domain to an extracellular signaling domain of a costimulatory immune cell ligand. Certain additional hybrid proteins described herein include from N-terminal to C-terminal expression, an inverted intracellular signaling domain of a costimulatory immune cell signaling domain to a transmembrane domain to an extracellular signaling domain of a costimulatory immune cell ligand.

In additional embodiments, expression of hybrid and truncated proteins disclosed herein can be modulated to further refine immune system activation and diversification. Such modulation can be achieved by including a chemically-induced multimerization sequence segment in the proteins and/or by controlling expression of the proteins using an inducible expression system.

Cells genetically modified to express a hybrid and/or truncated protein disclosed herein can also be genetically modified to express another protein with a therapeutic purpose such as a CAR or a recombinant TCR.

Additional aspects of the disclosure are now described as follows: (i) Chemically-Induced Multimerization Systems (CIDS); (ii) Genes & Gene Delivery Techniques; (iii) Ex Vivo Cell Manufacturing; (iv) Nanoparticles for In Vivo Nucleotide Delivery to Immune Cells; (v) Methods of Use; (vi) Exemplary Embodiments; and (vii) Closing Paragraphs. These headings are provided for organization purposes only and do not limit the scope or interpretation of the disclosure.

(i) Chemically-Induced Multimerization Systems (CIMS). In particular embodiments, a chemical inducer of multimerization (CIM) (e.g., dimerization, trimerization) may be used within hybrid or truncated proteins described herein. The CIM and CIM binding domains may be any combination of molecules, peptides, or domains which enable the selective co-localization and multimerization of hybrid and/or truncated proteins in the presence of the CIM. For example, in particular embodiments, one hybrid protein includes a CIM binding domain 1 (CBM1) and the second hybrid protein contains the second CIM binding domain (CBM2). CBM1 and CBM2 are capable of simultaneously binding to the CIM. The CIM may interact with CBMs in which CBM1 and CBM2 are identical or the CIM may interact with two different CBMs so that CBM1 and CBM2 are not identical.

Small molecule CIMS for facilitating the co-localization of proteins are known in the art (Corson et al.; 2008; ACS Chemical Biology; 3(11); 667). The CIM and CBMs may be the FK506 binding protein (FKBP) ligand dimerization system described by Clackson et al. (PNAS; 1998; 95; 10437-10442). This dimerization system includes two FKBP-like binding domains with a F36V mutation in the FKBP binding domain and a CID dimerization agent (AP1903) with complementary amino acid substitutions. Exposing cells engineered to express FKBP-like binding domain fusion proteins to AP103 results in the dimerization of the proteins including the FKBP-like binding domains but no interactions involving endogenous FKBP.

The CIM/CIM binding domain may also be the rapamycin and FKBP12/FKBP12-Rapamycin Binding (FRB) domain of the mTOR system described by Rivera et al. (Nature Med; 1996; 2; 1028-1032) or the non-immunosuppressive rapamycin analogs (rapalogs) and FKBP12/FRB system described by Bayle et al. (Chem Bio; 2006; 13; 99-107). For example the CIM may be C-20-methyllyrlrapamycin (MaRap) or C16(S)-Butylsulfonamidorapamycin (C16-BS-Rap). The CIM may be C16-(S)-3-methylindolerapamycin (C16-iRap) or C16-(S)-7-methylindolerapamycin (AP21976/C16-AiRap) in combination with the respective complementary binding domains for each.

The CIM and CBMs may include the dimerization system described by Belshaw et al. (Nature; 1996; 93; 4604-4607), which utilizes a FK506 (Tacrolimus)/cyclosporin fusion molecule as the CIM agent with FK-binding protein 12 (FKBP12) and cylcophilin A as the CBMs.

Bacterial DNA gyrase B (GyrB) binding domains can be used as CBMs within a dimerization system with the antibiotic coumermycin as the CIM (Farrar et al., Methods Enzymol; 2000; 327; 421-419 and Nature; 1996; 383; 178-181).

Other dimerization systems include an estrone/biotin CIM in combination with an oestrogen-binding domain (EBD) and a streptavidin binding domain (Muddana & Peterson; Org. Lett; 2004; 6; 1409-1412; Hussey et al.; J. Am. Chem. Soc.; 125; 3692-3693) and a dexamethasone/methotrexate CIM in combination with a glucocorticoid-binding domain (GBD) and a dihydrofolate reductase (DHFR) binding domain (Lin et al.; J. Am. Chem. Soc.; 2000; 122; 4247-4248). RSL1 or a derivative thereof can be used as a CIM in the heterodimerization of molecules with CBMs made up of EcR and RXR domains. Other examples of dimerization systems include a CIM in which the methotrexate portion of the CIM is replaced with the bacterial specific DHFR inhibitor trimethoprim (Gallagher et al.; Anal. Biochem; 2007; 363; 160-162) and an O⁶ -benzylguanine derivative/methotrexate CIM in combination with an O6-alkylguanine-DNA alkyltransferase (AGT) binding domain and a DHFR binding domain (Gendreizig et al.; J. Am. Chem. Soc.; 125; 14970-14971).

Trimerization systems can be engineered similarly to dimerization systems. For example, a chemically inducible trimerization domain can be engineered by splitting FRB and/or FKBP. Efficient trimerization of split pairs of FRB or FKBP with full-length FKBP or FRB, respectively by rapamycin is described in Wu, et al., Nature Methods 17, 928-936, 2020.

(ii) Genes & Gene Delivery Techniques. Immune cells are modified to express a hybrid and/or truncated protein of the disclosure by delivering a nucleotide including a gene that encodes the hybrid and/or truncated protein. A gene is a distinct sequence of nucleotides, the order of which determines the order of monomers in a polypeptide or nucleic acid molecule which a cell (or virus) may synthesize. The term “gene” may include not only coding sequences but also regulatory regions such as promoters, enhancers, and termination regions.

A promoter is a region of DNA, generally upstream (5′) of a coding region, which controls at least in part the initiation and level of gene transcription. Promoters generally extend upstream from a transcription initiation site and are involved in the binding of RNA polymerase. Promoters may contain several short («10 base pair) sequence elements that bind transcription factors, generally dispersed over »200 base pairs. The term promoter includes inducible and constitutive promoters. Particular embodiments disclosed herein utilize inducible promoters.

An inducible promoter refers to a promoter whose activity can be increased or decreased upon an external stimulus. Stimuli can be chemical or physical in nature, such as by administration of a chemical or by adjustment of temperature or light.

Chemically inducible promoters include reproductive hormone induced promoters and antibiotic inducible promoters such as the tetracycline inducible promoter and the zinc-inducible metallothionine promoter.

An example of a chemically inducible system includes the Tet-Off™ or Tet-On™ system (Clontech, Palo Alto, Calif.). This system allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli.

Other inducible promoter systems include the Lac operator repressor system inducible by IPTG (isopropyl beta-D-thiogalactoside) (Cronin, A. et al. 2001. Genes and Development, v. 15); ecdysone-based inducible systems (Hoppe, U. C. et al. 2000. Mol. Ther. 1:159-164); estrogen-based inducible systems (Braselmann, S. et al. 1993. Proc. Natl. Acad. Sci. 90:1657-1661); progesterone-based inducible systems; and CID-based inducible systems. A progesterone-based inducible system, for example, uses a chimeric regulator, GLVP, which is a hybrid protein including the GAL4 binding domain and the herpes simplex virus transcriptional activation domain, VP16, and a truncated form of the human progesterone receptor that retains the ability to bind ligand and can be turned on by RU486 (Wang, et al. 1994. Proc. Natl. Acad. Sci. 91:8180-8184). As indicated previously, a CID-based inducible system, for example, uses CIDs to regulate gene expression, such as a system wherein rapamycin induces dimerization of the cellular proteins FKBP12 and FRAP (Belshaw, P. J. et al. 1996. J. Chem. Biol. 3:731-738; Fan, L. et al. 1999. Hum. Gene Ther. 10:2273-2285; Shariat, S. F. et al. 2001. Cancer Res. 61:2562-2571; Spencer, D. M. 1996. Curr. Biol. 6:839-847). Chemical substances that activate the chemically inducible promoters can be administered to a cell or subject containing the gene of interest via any method known to those of skill in the art.

Temperature inducible are induced to prompt expression with exposure to either heat or cold. Temperature inducible promoters include heat shock-inducible Hsp70 or Hsp90-derived promoters which prompt expression due to a brief heat shock.

Light inducible promoters use light to regulate transcription. For example, red flame plasmid pDawn contains the blue-light sensing protein YFI. When light is present, YFI is inactive. Without light, YFI phosphorylates FixJ, which binds to the FixK2 promoter to induce transcription of the phage repressor cl, inhibiting transcription from phage promoter pR to prevent expression of a reporter gene.

Constitutive promoters are unregulated promoters that allow for continual transcription of genes. Constitutive promoters include immediate early cytomegalovirus (CMV) promoter, herpes simplex virus 1 (HSV1) immediate early promoter, SV40 promoter, lysozyme promoter, early and late CMV promoters, early and late HSV promoters, β-actin promoter, tubulin promoter, Rous-Sarcoma virus (RSV) promoter, and heat-shock protein (HSP) promoter.

An enhancer is a cis-acting sequence that increases the level of transcription associated with a promoter, and can function in either orientation relative to the promoter and the coding sequence that is to be transcribed, and can be located upstream or downstream relative to the promoter or the coding sequence to be transcribed.

Other regulatory elements include: silencers, insulators, and locus control regions. Silencers are opposite to enhancers in that they are capable of binding transcription regulation factors call repressors and thus reduce the level of transcription of a target gene. Insulators limit inappropriate interactions between adjacent domains. Insulators prevent regulatory elements from having an effect on domains outside the domain of the regulatory element. Locus control regions (LCRs) are long-range cis-regulatory elements that control expression of an entire set of linked genes on a locus.

Nucleotides with genes encoding hybrid proteins can be delivered to immune cells using any technique known to those of ordinary skill in the art, such as through electroporation, viral vectors, and nanoparticles.

In particular embodiments, a vector can be used to deliver nucleotides to cells. A vector is any nucleic acid vehicle (DNA or RNA) capable of facilitating the transfer of a nucleotide of interest into cells. In general, vectors include plasmids, phagemids, viral vectors, and other vehicles derived from viral or bacterial sources that have been manipulated by the insertion or incorporation of a target nucleotide sequence.

In particular embodiments, nucleotides can be delivered by electroporation in which an electrical field is applied to cells in order to increase their permeability. Instruments that can be used for electroporation include the Neon transfection system (Thermo Fisher Scientific, Waltham, MA), Gemini instrument and AgilePulse/CytoPulse instrument (BTX-Harvard apparatus, Holliston, MA), 4D-Nucleofector system, Amaxa Nucleofector II, Nucleofector 2b instrument (Lonza, Switzerland), CTX-1500A instrument (Celetrix, Manassas, VA), MaxCyte GT or VLX instrument (MaxCyte, Gathersbur, MD), and Gene Pulser Xcell (Biorad, Hercules, CA).

In particular embodiments, viral vectors can be used to deliver nucleotides to immune cells. Viral vectors can include any non-cytopathic eukaryotic virus in which nonessential genes have been replaced with the target nucleotide sequence to be delivered. Non-cytopathic viruses include lentivirus; adenovirus; adeno-associated virus (AAV); SV40-type viruses; polyoma viruses; Epstein-Barr viruses; papilloma viruses; herpes virus; vaccinia virus; and polio virus. One can readily employ other vectors not named but known to the art.

Nanoparticles can be used to selectively deliver nucleotides to immune cells ex vivo or in vivo, as described more fully below. Targeted nanoparticles capable of in vivo delivery can take many forms and will generally include a cell-specific targeting ligand (e.g., derived from an antibody binding domain that binds a marker of an immune-targeted cell). For example, all T cells express CD3 whereas helper T cells express CD4 and cytotoxic T cells express CD8+. Numerous additional immune cell surface markers are known to those of ordinary skill and the art and can be used for targeted nanoparticle delivery.

Nanoparticles can be formed in a variety of different shapes, including spheroidal, cuboidal, pyramidal, oblong, cylindrical, toroidal, and the like. Nucleotides can be encapsulated within nanoparticles and/or covalently and/or non-covalently bound to the surface or close underlying vicinity of the surface of the nanoparticle.

Liposomes are microscopic vesicles including at least one concentric lipid bilayer that surrounds an aqueous core. In certain examples, the structure of a liposome can be used to encapsulate a nanoparticle within its core (i.e., a liposomal nanoparticle). Lipid nanoparticles (LNPs) are liposome-like structures that lack the continuous lipid bilayer characteristic of liposomes. Solid lipid nanoparticles (SLNs) are LNPs that are solid at room and body temperatures. Liposomes and similar structures can be neutral (cholesterol) or bipolar and include phospholipids.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986).

Nanoparticles can also be formulated from configurations of positively-charged and neutral or negatively-charged polymers. Examples of positively charged polymers include polyamines; polyorganic amines (e.g., polyethyleneimine (PEI), polyethyleneimine celluloses); poly(amidoamines) (PAMAM); and polyamino acids (e.g., polylysine (PLL), polyarginine).Examples of neutrally charged polymers include polyethylene glycol (PEG); poly(propylene glycol); and polyalkylene oxide copolymers, (PLURONIC®, BASF Corp., Mount Olive, NJ).

Blends of polymers in any concentration and in any ratio can also be used. Blending different polymer types in different ratios using various grades can result in characteristics that borrow from each of the contributing polymers. Various terminal group chemistries can also be adopted.

The size of nanoparticles can vary and can be measured in different ways. In certain examples, nanoparticles have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm.

Particular embodiments may also use targeted genetic engineering systems to insert delivered nucleotides into a targeted region of the genome. Such systems include the CRISPR (Clustered Regularly Interspaced Short Palindromic Repeats)/Cas (CRISPR-associated protein) nuclease system, zinc finger nucleases (ZFNs), transcription activator-like effector nulceases (TALENs), or MegaTALs having a single chain rare-cleaving nuclease structure in which a TALE is fused with the DNA cleavage domain of a meganuclease.

(iii) Ex Vivo Cell Manufacturing. Immune cells can be obtained from a number of sources, including peripheral blood, mobilized peripheral blood, bone marrow, lymph node tissue, spleen tissue, and tumors. In certain embodiments, Immune cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis or leukapheresis product typically contains lymphocytes, including T cells, monocytes, granulocytes, B cells, other nucleated white blood cells, red blood cells, and platelets. In one embodiment, the cells collected by apheresis may be washed to remove the plasma fraction and the cells may be placed in an appropriate buffer or media for subsequent processing. In one embodiment, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

A specific subpopulation of immune cells can be isolated by positive or negative selection techniques. For example, immune cells can be isolated by incubation with antibody-conjugated beads (e.g., specific for any marker described herein), such as DYNABEADS® (Life Technologies AS, Oslo, Norway) for a time period sufficient for positive selection of the desired immune cells. In particular embodiments, the time period ranges from 30 minutes to 36 hours. In particular embodiments, the time period is 30 minutes, 1 hour, 2 hours, 3 hours, 4 hours, 5 hours, or 6 hours. Longer incubation times may be used to isolate immune cells in any situation where there are few immune cells as compared to other cell types, such in isolating tumor infiltrating lymphocytes (TIL) from tumor tissue or from immunocompromised individuals. Further, use of longer incubation times can increase the efficiency of capture of CD8+ T cells. Immune cells can be selected based on a biomarker on the cell surface including CD3, CD8, TIM-3, LAG-3, 4-1BB, or PD-1.

Enrichment of an immune cell population by positive or negative selection can be accomplished with a combination of antibodies directed to surface markers unique to the positively or negatively selected cells. One method is to use cell sorting and/or selection via negative magnetic immunoadherence or flow cytometry that uses a cocktail of monoclonal antibodies directed to cell surface markers present on the cells negatively selected. Sorting of immune cells, or generally any cells, can be carried out using any of a variety of commercially available cell sorters, including MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

The efficiency of the purification can be analyzed by flow cytometry (Coulter, EPICS Elite), using, for example, anti-CD3, anti-CD4, anti-CD8, anti-CD14 mAbs or additional antibodies that recognize specific subsets of T cells, followed by fluorescein isothiocyanate conjugated goat anti mouse immunoglobulin (Fisher, Pittsburgh, PA) or other secondary antibody.

The isolated cells can be expanded in a culture media under specific conditions. In particular embodiments, the isolated cells are cultured in the presence of IL-2. In particular embodiments, the isolated cells can be expanded using methods described in U.S. Pat. No. 8,637,307. The numbers of immune cells may be increased at least 3-fold (or 4-, 5-, 6-, 7-, 8-, or 9-fold), more preferably at least 10-fold (or 20-, 30-, 40-, 50-, 60-, 70-, 80-, or 90-fold), more preferably at least 100-fold, more preferably at least 1,000 fold, or most preferably at least 100,000-fold. The numbers of immune cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133.

Expanded cells may be activated in culture utilizing appropriate stimulating ligands (e.g., with CD3/CD28 beads useful for stimulating the CD3 primary signal and the CD28 accessory or co-stimulatory signal). Activating ligands may be soluble or immobilized on a surface.

Exemplary carriers for cell formulations for administration to a subject include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof.

Therapeutically effective amounts of cells within cell-based formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cell formulations are administered to subjects as soon as reasonably possible following their initial formulation. In particular embodiments, immune cells can be frozen or cryopreserved. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. Prior to administration to a subject, the engineered cells can be thawed.

(iv) Nanoparticles for In Vivo Nucleotide Delivery to Immune Cells. As described previously, nanoparticles can be used to selectively deliver nucleotides to immune cells in vivo. In this scenario, nanoparticles can be formulated into compositions for delivery with a pharmaceutically acceptable carrier that is suitable for administration to a subject. Pharmaceutically acceptable carriers include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

Therapeutically effective amounts of nanoparticles within a composition can include at least 0.1% w/v or w/w particles; at least 1% w/v or w/w particles; at least 10% w/v or w/w particles; at least 20% w/v or w/w particles; at least 30% w/v or w/w particles; at least 40% w/v or w/w particles; at least 50% w/v or w/w particles; at least 60% w/v or w/w particles; at least 70% w/v or w/w particles; at least 80% w/v or w/w particles; at least 90% w/v or w/w particles; at least 95% w/v or w/w particles; or at least 99% w/v or w/w particles.

(v) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with ex vivo manufactured cell formulations or nanoparticle compositions disclosed herein. Treating subjects includes delivering therapeutically effective amounts. Therapeutically effective amounts include those that provide effective amounts, prophylactic treatments and/or therapeutic treatments.

An “effective amount” is the amount of a formulation or composition necessary to result in a desired physiological effect. Effective amounts are often administered for research purposes. Effective amounts disclosed herein can cause an indication of immune cell activation in an in vitro assessment or within an in vivo animal model. Indications of T cell activation include cytokine release, upregulated activation (e.g. expression of CD69, CD25, etc.), tumor cell lysis, proliferation, and accumulation.

A “prophylactic treatment” includes a treatment administered to a subject who does not display signs or symptoms of a condition (e.g., cancer or an infection) or displays only early signs or symptoms of the condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” can be liquid or solid depending on the cell origin. A solid tumor is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease and can be considered a solid tumor or liquid tumor in the art depending on the cell origin. Tumors show partial or complete lack of structural organization and functional coordination with the normal tissue, and usually form a distinct mass of tissue, which may be benign, pre-malignant or malignant. Liquid tumors refer to the total mass of circulating neoplastic cells, for examples in hematopoietic malignancies such as leukemia.

In particular embodiments, therapeutically effective amounts provide anti-infection effects. Infections may be viral, bacterial, fungal, protozoan, parasitic, or prion infections. Examples of viral diseases include measles, rubella, COVID-19, chickenpox/shingles, roseola, smallpox, and influenza. Examples of bacteria that cause infections include Streptococcus, Staphylococcus, Tuberculosis, Salmonella, and Escherichia coli. Examples of fungal infections include Histoplasmosis, Blastomycosis, Coccidioidomycosis, Paracoccidioidomycosis, Aspergillosis, Candidiasis, and Mucormycosis. Examples of parasitic infections include toxoplasmosis, giardiasis, cryptosporidiosis, and trichomoniasis. Examples of prion diseases include Creutzfeldt-Jakob Disease, Variant Creutzfeldt-Jakob Disease, Fatal Familial Insomnia, Kuru, Gerstmann-Straussler-Schneinker Syndrome, Bovine Spongiform, and Chronic Wasting Disease.

In particular embodiments, modified cells described herein may be used for adoptive cell transfer (ACT). Adoptive cell transfer can include: isolating from a biological sample of the subject an immune cell or immune cell population; in vitro expanding and modifying the immune cell or immune cell population to express a gene (e.g., a gene encoding a hybrid and/or truncated protein described herein and optionally one or more additional therapeutic molecules); and administering the in vitro expanded/modified immune cell or immune cell population to the subject. The method may further include enriching the expanded immune cells for one subtype. In certain embodiments, the method may further include formulating the in vitro expanded immune cell or immune cell population into a cell-based formulation. In this manner, ACT refers to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

In certain embodiments, cells modified to express a hybrid and/or truncated protein disclosed herein provide an anti-cancer or anti-infection treatment by providing enhanced immune system activation. In certain embodiments, cells modified to express a protein disclosed herein provide an anti-cancer or anti-infection treatment in combination with a cancer treatment or infection treatment.

In certain embodiments, a cell genetically modified to express a hybrid and/or truncated protein disclosed herein is additionally genetically modified to express a CAR or TCR. The CAR or TCR can bind a cancer antigen or a viral antigen.

Exemplary cancer antigens include bladder cancer antigens: MUC16, PD-L1, EGFR;

breast cancer antigens: HER2, ERBB2, ROR1, PD-L1, EGFR, MUC16, FOLR, CEA, p53; cholangiocarcinoma antigens: mesothelin, PD-L1, EGFR; colorectal cancer antigens: CEA, PD-L1, EGFR, K-ras; glioblastoma antigens: EGFR variant III (EGFRvIII), IL13Ra2; lung cancer antigens: ROR1, PD-L1, EGFR, mesothelin, MUC16, FOLR, CEA, CD56, p53, Kras; Merkel cell carcinoma antigens: CD56, PD-L1, EGFR; mesothelioma antigens: mesothelin, PD-L1, EGFR; neuroblastoma antigens: ROR1, glypican-2, CD56, disialoganglioside, PD-L1, EGFR; ovarian cancer antigens: EpCam, L1-CAM, MUC16, folate receptor (FOLR), Lewis Y, ROR1, mesothelin, WT-1, PD-L1, EGFR, CD56, p53; melanoma antigens: Tyrosinase related protein 1 (TYRP1/gp75); GD2, PD-L1, EGFR; multiple myeloma antigens: B-cell maturation antigen (BCMA), PD-L1, EGFR; pancreatic cancer antigens: mesothelin, CEA, CD24, ROR1, PD-L1, EGFR, MUC16, K-ras; prostate cancer antigens: PSMA, VVT1, Prostate Stem Cell antigen (PSCA), SV40 T, PD-L1, EGFR; renal cell carcinoma antigens: carboxy-anhydrase-IX (CAIX); PD-L1, EGFR; and stem cell cancer antigens: CD133, PD-L1, EGFR.

Exemplary viral antigens include coronaviral antigens: the spike (S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins El and E2; and varicella zoster viral antigens: gpI and gpII. See Fundamental Virology, Second Edition, eds. Fields, B. N. and Knipe, D. M. (Raven Press, New York, 1991) for additional examples of viral antigens.

### (vi) Exemplary Embodiments

1. A protein including
   an extracellular domain of OX40L;
   an intracellular domain of CD30, HVEM, or CD40; and
   a transmembrane domain linking the extracellular domain to the intracellular domain.
2. A protein including an extracellular domain of a stimulatory immune cell protein, an intracellular domain of a different stimulatory immune cell protein, and a transmembrane domain linking the extracellular domain to the intracellular domain.
3. The protein of embodiment 2, wherein expression of the protein by an immune cell increases activation of the immune cell in the presence of a ligand, when compared to an immune cell that does not express the protein in the presence of the same ligand.
4. The protein of embodiments 2 or 3, wherein the extracellular domain is an extracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.
5. The protein of any of embodiments 2-4, wherein the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.
6. The protein of any of embodiments 2-5, wherein
   the extracellular domain is an extracellular domain of 4-1BB and the intracellular domain is an intracellular domain of 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.
   the extracellular domain is an extracellular domain of 4-1BBL and the intracellular domain is an intracellular domain of 4-1BB, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of OX40 and the intracellular domain is an intracellular domain of 4-1BB, 4-1BBL, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of OX40L and the intracellular domain is an intracellular domain of 4-1BB, 4-1BBL, OX40, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD40 and the intracellular domain is an intracellular domain of 4-1BB, 4-1BBL, OX40, OX40L, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD40L and the intracellular domain is an intracellular domain of 4-1BB, 4-1BBL, OX40, OX40L, CD40, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD30 and the intracellular domain is an intracellular domain of 4-1BB, 4-1BBL, OX40, OX40L, CD40, CD40L, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD30L and the intracellular domain is an intracellular domain of 4-1BB, 4-1BBL, OX40, OX40L, CD40, CD40L, CD30, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD27 and the intracellular domain is an intracellular domain of 4-1BB, 4-1BBL, OX40, OX40L, CD40, CD40L, CD30, CD30L, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of CD27L and the intracellular domain is an intracellular domain of 4-1BB, 4-1BBL, OX40, OX40L, CD40, CD40L, CD30, CD30L, CD27, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of DR3 and the intracellular domain is an intracellular domain of 4-1BB, 4-1BBL, OX40, OX40L, CD40, CD40L, CD30, CD30L, CD27, CD27L, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of DR3L and the intracellular domain is an intracellular domain of 4-1BB, 4-1BBL, OX40, OX40L, CD40, CD40L, CD30, CD30L, CD27, CD27L, DR3, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of FLT3 and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
   the extracellular domain is an extracellular domain of HVEM and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L; or
   the extracellular domain is an extracellular domain of LIGHT and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCR8, TIM1, TIM1L, TRIM, TRIML, Zap70, Zap70L, PTCH2, or PTCH2L.
7. The protein of any of embodiments 2-6, further including at least a second copy of the intracellular domain, the second copy also linked to the extracellular domain through the transmembrane domain.
8. The protein of any of embodiments 2-7, further including the intracellular domain of the stimulatory immune cell protein.
9. The protein of embodiment 8, wherein the stimulatory immune cell protein includes 4-1BB, CD40, or OX40.
10. The protein of any of embodiments 2-9, further including an intracellular domain of a third stimulatory immune cell protein.
11. The protein of embodiment 10, wherein the third stimulatory immune cell protein includes 4-1BB, CD40, or OX40.
12. The protein of any of embodiments 2-11, wherein the stimulatory immune cell protein and the different immune stimulatory protein both include dimerizing intracellular domains.
13. The protein of any of embodiments 2-12, wherein
   the extracellular domain includes an extracellular domain of CD2 and the intracellular domain includes an intracellular domain of CD28;
   the extracellular domain includes an extracellular domain of CD2 and the intracellular domain includes an intracellular domain of CD226;
   the extracellular domain includes an extracellular domain of CD2 and the intracellular domain includes an intracellular domain of CRTAM;
   the extracellular domain includes an extracellular domain of CD2 and the intracellular domain includes an intracellular domain of TIM1;
   the extracellular domain includes an extracellular domain of CD2 and the intracellular domain includes an intracellular domain of ICOS;
   the extracellular domain includes an extracellular domain of CD2 and the intracellular domain includes an intracellular domain of SLAMF1;
   the extracellular domain includes an extracellular domain of CD2 and the intracellular domain includes an intracellular domain of Ly9;
   the extracellular domain includes an extracellular domain of CD2 and the intracellular domain includes an intracellular domain of CD84;
   the extracellular domain includes an extracellular domain of CD2 and the intracellular domain includes an intracellular domain of SLAMF7;
   the extracellular domain includes an extracellular domain of CD2 and the intracellular domain includes an intracellular domain of BLAME;
   the extracellular domain includes an extracellular domain of CD28 and the intracellular domain includes an intracellular domain of CD2;
   the extracellular domain includes an extracellular domain of CD28 and the intracellular domain includes an intracellular domain of CD226;
   the extracellular domain includes an extracellular domain of CD28 and the intracellular domain includes an intracellular domain of CRTAM;
   the extracellular domain includes an extracellular domain of CD28 and the intracellular domain includes an intracellular domain of TIM1;
   the extracellular domain includes an extracellular domain of CD28 and the intracellular domain includes an intracellular domain of ICOS;
   the extracellular domain includes an extracellular domain of CD28 and the intracellular domain includes an intracellular domain of SLAMF1;
   the extracellular domain includes an extracellular domain of CD28 and the intracellular domain includes an intracellular domain of Ly9;
   the extracellular domain includes an extracellular domain of CD28 and the intracellular domain includes an intracellular domain of CD84;
   the extracellular domain includes an extracellular domain of CD28 and the intracellular domain includes an intracellular domain of SLAMF7;
   the extracellular domain includes an extracellular domain of CD28 and the intracellular domain includes an intracellular domain of BLAME;
   the extracellular domain includes an extracellular domain of CD226 and the intracellular domain includes an intracellular domain of CD2;
   the extracellular domain includes an extracellular domain of CD226 and the intracellular domain includes an intracellular domain of CD28;
   the extracellular domain includes an extracellular domain of CD226 and the intracellular domain includes an intracellular domain of CRTAM;
   the extracellular domain includes an extracellular domain of CD226 and the intracellular domain includes an intracellular domain of TIM1;
   the extracellular domain includes an extracellular domain of CD226 and the intracellular domain includes an intracellular domain of ICOS;
   the extracellular domain includes an extracellular domain of CD226 and the intracellular domain includes an intracellular domain of SLAMF1;
   the extracellular domain includes an extracellular domain of CD226 and the intracellular domain includes an intracellular domain of Ly9;
   the extracellular domain includes an extracellular domain of CD226 and the intracellular domain includes an intracellular domain of CD84;
   the extracellular domain includes an extracellular domain of CD226 and the intracellular domain includes an intracellular domain of SLAMF7;
   the extracellular domain includes an extracellular domain of CD226 and the intracellular domain includes an intracellular domain of BLAME;
   the extracellular domain includes an extracellular domain of CRTAM and the intracellular domain includes an intracellular domain of CD2;
   the extracellular domain includes an extracellular domain of CRTAM and the intracellular domain includes an intracellular domain of CD28;
   the extracellular domain includes an extracellular domain of CRTAM and the intracellular domain includes an intracellular domain of CD226;
   the extracellular domain includes an extracellular domain of CRTAM and the intracellular domain includes an intracellular domain of TIM1;
   the extracellular domain includes an extracellular domain of CRTAM and the intracellular domain includes an intracellular domain of ICOS;
   the extracellular domain includes an extracellular domain of CRTAM and the intracellular domain includes an intracellular domain of SLAMF1;
   the extracellular domain includes an extracellular domain of CRTAM and the intracellular domain includes an intracellular domain of Ly9;
   the extracellular domain includes an extracellular domain of CRTAM and the intracellular domain includes an intracellular domain of CD84;
   the extracellular domain includes an extracellular domain of CRTAM and the intracellular domain includes an intracellular domain of SLAMF7;
   the extracellular domain includes an extracellular domain of CRTAM and the intracellular domain includes an intracellular domain of BLAME;
   the extracellular domain includes an extracellular domain of TIM1 and the intracellular domain includes an intracellular domain of CD2;
   the extracellular domain includes an extracellular domain of TIM1 and the intracellular domain includes an intracellular domain of CD28;
   the extracellular domain includes an extracellular domain of TIM1 and the intracellular domain includes an intracellular domain of CD226;
   the extracellular domain includes an extracellular domain of TIM1 and the intracellular domain includes an intracellular domain of CRTAM;
   the extracellular domain includes an extracellular domain of TIM1 and the intracellular domain includes an intracellular domain of ICOS;
   the extracellular domain includes an extracellular domain of TIM1 and the intracellular domain includes an intracellular domain of SLAMF1;
   the extracellular domain includes an extracellular domain of TIM1 and the intracellular domain includes an intracellular domain of Ly9;
   the extracellular domain includes an extracellular domain of TIM1 and the intracellular domain includes an intracellular domain of CD84;
   the extracellular domain includes an extracellular domain of TIM1 and the intracellular domain includes an intracellular domain of SLAMF7;
   the extracellular domain includes an extracellular domain of TIM1 and the intracellular domain includes an intracellular domain of BLAME;
   the extracellular domain includes an extracellular domain of ICOS and the intracellular domain includes an intracellular domain of CD2;
   the extracellular domain includes an extracellular domain of ICOS and the intracellular domain includes an intracellular domain of CD28;
   the extracellular domain includes an extracellular domain of ICOS and the intracellular domain includes an intracellular domain of CD226;
   the extracellular domain includes an extracellular domain of ICOS and the intracellular domain includes an intracellular domain of CRTAM;
   the extracellular domain includes an extracellular domain of ICOS and the intracellular domain includes an intracellular domain of TIM1;
   the extracellular domain includes an extracellular domain of ICOS and the intracellular domain includes an intracellular domain of SLAMF1;
   the extracellular domain includes an extracellular domain of ICOS and the intracellular domain includes an intracellular domain of Ly9;
   the extracellular domain includes an extracellular domain of ICOS and the intracellular domain includes an intracellular domain of CD84;
   the extracellular domain includes an extracellular domain of ICOS and the intracellular domain includes an intracellular domain of SLAMF7;
   the extracellular domain includes an extracellular domain of ICOS and the intracellular domain includes an intracellular domain of BLAME;
   the extracellular domain includes an extracellular domain of SLAMF1 and the intracellular domain includes an intracellular domain of CD2;
   the extracellular domain includes an extracellular domain of SLAMF1 and the intracellular domain includes an intracellular domain of CD28;
   the extracellular domain includes an extracellular domain of SLAMF1 and the intracellular domain includes an intracellular domain of CD226;
   the extracellular domain includes an extracellular domain of SLAMF1 and the intracellular domain includes an intracellular domain of CRTAM;
   the extracellular domain includes an extracellular domain of SLAMF1 and the intracellular domain includes an intracellular domain of TIM1;
   the extracellular domain includes an extracellular domain of SLAMF1 and the intracellular domain includes an intracellular domain of ICOS;
   the extracellular domain includes an extracellular domain of SLAMF1 and the intracellular domain includes an intracellular domain of Ly9;
   the extracellular domain includes an extracellular domain of SLAMF1 and the intracellular domain includes an intracellular domain of CD84;
   the extracellular domain includes an extracellular domain of SLAMF1 and the intracellular domain includes an intracellular domain of SLAMF7;
   the extracellular domain includes an extracellular domain of SLAMF1 and the intracellular domain includes an intracellular domain of BLAME;
   the extracellular domain includes an extracellular domain of Ly9 and the intracellular domain includes an intracellular domain of CD2;
   the extracellular domain includes an extracellular domain of Ly9 and the intracellular domain includes an intracellular domain of CD28;
   the extracellular domain includes an extracellular domain of Ly9 and the intracellular domain includes an intracellular domain of CD226;
   the extracellular domain includes an extracellular domain of Ly9 and the intracellular domain includes an intracellular domain of CRTAM;
   the extracellular domain includes an extracellular domain of Ly9 and the intracellular domain includes an intracellular domain of TIM1;
   the extracellular domain includes an extracellular domain of Ly9 and the intracellular domain includes an intracellular domain of ICOS;
   the extracellular domain includes an extracellular domain of Ly9 and the intracellular domain includes an intracellular domain of SLAMF1;
   the extracellular domain includes an extracellular domain of Ly9 and the intracellular domain includes an intracellular domain of CD84;
   the extracellular domain includes an extracellular domain of Ly9 and the intracellular domain includes an intracellular domain of SLAMF7;
   the extracellular domain includes an extracellular domain of Ly9 and the intracellular domain includes an intracellular domain of BLAME;
   the extracellular domain includes an extracellular domain of CD84 and the intracellular domain includes an intracellular domain of CD2;
   the extracellular domain includes an extracellular domain of CD84 and the intracellular domain includes an intracellular domain of CD28;
   the extracellular domain includes an extracellular domain of CD84 and the intracellular domain includes an intracellular domain of CD226;
   the extracellular domain includes an extracellular domain of CD84 and the intracellular domain includes an intracellular domain of CRTAM;
   the extracellular domain includes an extracellular domain of CD84 and the intracellular domain includes an intracellular domain of TIM1;
   the extracellular domain includes an extracellular domain of CD84 and the intracellular domain includes an intracellular domain of ICOS;
   the extracellular domain includes an extracellular domain of CD84 and the intracellular domain includes an intracellular domain of SLAMF1;
   the extracellular domain includes an extracellular domain of CD84 and the intracellular domain includes an intracellular domain of Ly9;
   the extracellular domain includes an extracellular domain of CD84 and the intracellular domain includes an intracellular domain of SLAMF7;
   the extracellular domain includes an extracellular domain of CD84 and the intracellular domain includes an intracellular domain of BLAME;
   the extracellular domain includes an extracellular domain of SLAMF7 and the intracellular domain includes an intracellular domain of CD2;
   the extracellular domain includes an extracellular domain of SLAMF7 and the intracellular domain includes an intracellular domain of CD28;
   the extracellular domain includes an extracellular domain of SLAMF7 and the intracellular domain includes an intracellular domain of CD226;
   the extracellular domain includes an extracellular domain of SLAMF7 and the intracellular domain includes an intracellular domain of CRTAM;
   the extracellular domain includes an extracellular domain of SLAMF7 and the intracellular domain includes an intracellular domain of TIM1;
   the extracellular domain includes an extracellular domain of SLAMF7 and the intracellular domain includes an intracellular domain of ICOS;
   the extracellular domain includes an extracellular domain of SLAMF7 and the intracellular domain includes an intracellular domain of SLAMF1;
   the extracellular domain includes an extracellular domain of SLAMF7 and the intracellular domain includes an intracellular domain of Ly9;
   the extracellular domain includes an extracellular domain of SLAMF7 and the intracellular domain includes an intracellular domain of CD84;
   the extracellular domain includes an extracellular domain of SLAMF7 and the intracellular domain includes an intracellular domain of BLAME;
   the extracellular domain includes an extracellular domain of BLAME and the intracellular domain includes an intracellular domain of CD2;
   the extracellular domain includes an extracellular domain of BLAME and the intracellular domain includes an intracellular domain of CD28;
   the extracellular domain includes an extracellular domain of BLAME and the intracellular domain includes an intracellular domain of CD226;
   the extracellular domain includes an extracellular domain of BLAME and the intracellular domain includes an intracellular domain of CRTAM;
   the extracellular domain includes an extracellular domain of BLAME and the intracellular domain includes an intracellular domain of TIM1;
   the extracellular domain includes an extracellular domain of BLAME and the intracellular domain includes an intracellular domain of ICOS;
   the extracellular domain includes an extracellular domain of BLAME and the intracellular domain includes an intracellular domain of SLAMF1;
   the extracellular domain includes an extracellular domain of BLAME and the intracellular domain includes an intracellular domain of Ly9;
   the extracellular domain includes an extracellular domain of BLAME and the intracellular domain includes an intracellular domain of CD84; or
   the extracellular domain includes an extracellular domain of BLAME and the intracellular domain includes an intracellular domain of SLAMF7.
14. The protein of any of embodiments 2-12, wherein the stimulatory immune cell protein and the different immune stimulatory protein both include trimerizing intracellular domains.
15. The protein of embodiment 14, wherein the extracellular domain includes an extracellular domain of OX40 and the intracellular domain includes an intracellular domain of CD27;
   the extracellular domain includes an extracellular domain of OX40 and the intracellular domain includes an intracellular domain of CD30;
   the extracellular domain includes an extracellular domain of OX40 and the intracellular domain includes an intracellular domain of CD40;
   the extracellular domain includes an extracellular domain of OX40 and the intracellular domain includes an intracellular domain of HVEM;
   the extracellular domain includes an extracellular domain of OX40 and the intracellular domain includes an intracellular domain of DR3;
   the extracellular domain includes an extracellular domain of OX40 and the intracellular domain includes an intracellular domain of 41BB;
   the extracellular domain includes an extracellular domain of OX40 and the intracellular domain includes an intracellular domain of GITR;
   the extracellular domain includes an extracellular domain of OX40 and the intracellular domain includes an intracellular domain of LIGHT;
   the extracellular domain includes an extracellular domain of CD27 and the intracellular domain includes an intracellular domain of OX40;
   the extracellular domain includes an extracellular domain of CD27 and the intracellular domain includes an intracellular domain of CD30;
   the extracellular domain includes an extracellular domain of CD27 and the intracellular domain includes an intracellular domain of CD40;
   the extracellular domain includes an extracellular domain of CD27 and the intracellular domain includes an intracellular domain of HVEM;
   the extracellular domain includes an extracellular domain of CD27 and the intracellular domain includes an intracellular domain of DR3;
   the extracellular domain includes an extracellular domain of CD27 and the intracellular domain includes an intracellular domain of 41BB;
   the extracellular domain includes an extracellular domain of CD27 and the intracellular domain includes an intracellular domain of GITR;
   the extracellular domain includes an extracellular domain of CD27 and the intracellular domain includes an intracellular domain of LIGHT;
   the extracellular domain includes an extracellular domain of CD30 and the intracellular domain includes an intracellular domain of OX40;
   the extracellular domain includes an extracellular domain of CD30 and the intracellular domain includes an intracellular domain of CD27;
   the extracellular domain includes an extracellular domain of CD30 and the intracellular domain includes an intracellular domain of CD40;
   the extracellular domain includes an extracellular domain of CD30 and the intracellular domain includes an intracellular domain of HVEM;
   the extracellular domain includes an extracellular domain of CD30 and the intracellular domain includes an intracellular domain of DR3;
   the extracellular domain includes an extracellular domain of CD30 and the intracellular domain includes an intracellular domain of 41BB;
   the extracellular domain includes an extracellular domain of CD30 and the intracellular domain includes an intracellular domain of GITR;
   the extracellular domain includes an extracellular domain of CD30 and the intracellular domain includes an intracellular domain of LIGHT;
   the extracellular domain includes an extracellular domain of CD40 and the intracellular domain includes an intracellular domain of OX40;
   the extracellular domain includes an extracellular domain of CD40 and the intracellular domain includes an intracellular domain of CD27;
   the extracellular domain includes an extracellular domain of CD40 and the intracellular domain includes an intracellular domain of CD30;
   the extracellular domain includes an extracellular domain of CD40 and the intracellular domain includes an intracellular domain of HVEM;
   the extracellular domain includes an extracellular domain of CD40 and the intracellular domain includes an intracellular domain of DR3;
   the extracellular domain includes an extracellular domain of CD40 and the intracellular domain includes an intracellular domain of 41BB;
   the extracellular domain includes an extracellular domain of CD40 and the intracellular domain includes an intracellular domain of GITR;
   the extracellular domain includes an extracellular domain of CD40 and the intracellular domain includes an intracellular domain of LIGHT;
   the extracellular domain includes an extracellular domain of HVEM and the intracellular domain includes an intracellular domain of OX40;
   the extracellular domain includes an extracellular domain of HVEM and the intracellular domain includes an intracellular domain of CD27;
   the extracellular domain includes an extracellular domain of HVEM and the intracellular domain includes an intracellular domain of CD30;
   the extracellular domain includes an extracellular domain of HVEM and the intracellular domain includes an intracellular domain of CD40;
   the extracellular domain includes an extracellular domain of HVEM and the intracellular domain includes an intracellular domain of DR3;
   the extracellular domain includes an extracellular domain of HVEM and the intracellular domain includes an intracellular domain of 41BB;
   the extracellular domain includes an extracellular domain of HVEM and the intracellular domain includes an intracellular domain of GITR;
   the extracellular domain includes an extracellular domain of HVEM and the intracellular domain includes an intracellular domain of LIGHT;
   the extracellular domain includes an extracellular domain of DR3 and the intracellular domain includes an intracellular domain of OX40;
   the extracellular domain includes an extracellular domain of DR3 and the intracellular domain includes an intracellular domain of CD27;
   the extracellular domain includes an extracellular domain of DR3 and the intracellular domain includes an intracellular domain of CD30;
   the extracellular domain includes an extracellular domain of DR3 and the intracellular domain includes an intracellular domain of CD40;
   the extracellular domain includes an extracellular domain of DR3 and the intracellular domain includes an intracellular domain of HVEM;
   the extracellular domain includes an extracellular domain of DR3 and the intracellular domain includes an intracellular domain of 41BB;
   the extracellular domain includes an extracellular domain of DR3 and the intracellular domain includes an intracellular domain of GITR;
   the extracellular domain includes an extracellular domain of DR3 and the intracellular domain includes an intracellular domain of LIGHT;
   the extracellular domain includes an extracellular domain of 41BB and the intracellular domain includes an intracellular domain of OX40;
   the extracellular domain includes an extracellular domain of 41BB and the intracellular domain includes an intracellular domain of CD27;
   the extracellular domain includes an extracellular domain of 41BB and the intracellular domain includes an intracellular domain of CD30;
   the extracellular domain includes an extracellular domain of 41BB and the intracellular domain includes an intracellular domain of CD40;
   the extracellular domain includes an extracellular domain of 41BB and the intracellular domain includes an intracellular domain of HVEM;
   the extracellular domain includes an extracellular domain of 41BB and the intracellular domain includes an intracellular domain of DR3;
   the extracellular domain includes an extracellular domain of 41BB and the intracellular domain includes an intracellular domain of GITR;
   the extracellular domain includes an extracellular domain of 41BB and the intracellular domain includes an intracellular domain of LIGHT;
   the extracellular domain includes an extracellular domain of GITR and the intracellular domain includes an intracellular domain of OX40;
   the extracellular domain includes an extracellular domain of GITR and the intracellular domain includes an intracellular domain of CD27;
   the extracellular domain includes an extracellular domain of GITR and the intracellular domain includes an intracellular domain of CD30;
   the extracellular domain includes an extracellular domain of GITR and the intracellular domain includes an intracellular domain of CD40;
   the extracellular domain includes an extracellular domain of GITR and the intracellular domain includes an intracellular domain of HVEM;
   the extracellular domain includes an extracellular domain of GITR and the intracellular domain includes an intracellular domain of DR3;
   the extracellular domain includes an extracellular domain of GITR and the intracellular domain includes an intracellular domain of 41BB;
   the extracellular domain includes an extracellular domain of GITR and the intracellular domain includes an intracellular domain of LIGHT;
   the extracellular domain includes an extracellular domain of LIGHT and the intracellular domain includes an intracellular domain of OX40;
   the extracellular domain includes an extracellular domain of LIGHT and the intracellular domain includes an intracellular domain of CD27;
   the extracellular domain includes an extracellular domain of LIGHT and the intracellular domain includes an intracellular domain of CD30;
   the extracellular domain includes an extracellular domain of LIGHT and the intracellular domain includes an intracellular domain of CD40;
   the extracellular domain includes an extracellular domain of LIGHT and the intracellular domain includes an intracellular domain of HVEM;
   the extracellular domain includes an extracellular domain of LIGHT and the intracellular domain includes an intracellular domain of DR3;
   the extracellular domain includes an extracellular domain of LIGHT and the intracellular domain includes an intracellular domain of 41BB; or
   the extracellular domain includes an extracellular domain of LIGHT and the intracellular domain includes an intracellular domain of GITR.
16. The protein of any of embodiments 2-15, wherein:
   the extracellular domain includes an extracellular domain of 4-1BBL and the intracellular domain includes an intracellular domain of OX40;
   the extracellular domain includes an extracellular domain of 4-1BBL and the intracellular domain includes an intracellular domain of CD27;
   the extracellular domain includes an extracellular domain of 4-1BBL and the intracellular domain includes an intracellular domain of CD30;
   the extracellular domain includes an extracellular domain of 4-1BBL and the intracellular domain includes an intracellular domain of CD40;
   the extracellular domain includes an extracellular domain of 4-1BBL and the intracellular domain includes an intracellular domain of HVEM;
   the extracellular domain includes an extracellular domain of 4-1BBL and the intracellular domain includes an intracellular domain of DR3;
   the extracellular domain includes an extracellular domain of 4-1BBL and the intracellular domain includes an intracellular domain of 4-1BB;
   the extracellular domain includes an extracellular domain of OX40L and the intracellular domain includes an intracellular domain of OX40;
   the extracellular domain includes an extracellular domain of OX40L and the intracellular domain includes an intracellular domain of CD27;
   the extracellular domain includes an extracellular domain of OX40L and the intracellular domain includes an intracellular domain of CD30;
   the extracellular domain includes an extracellular domain of OX40L and the intracellular domain includes an intracellular domain of CD40;
   the extracellular domain includes an extracellular domain of OX40L and the intracellular domain includes an intracellular domain of HVEM;
   the extracellular domain includes an extracellular domain of OX40L and the intracellular domain includes an intracellular domain of DR3;
   the extracellular domain includes an extracellular domain of OX40L and the intracellular domain includes an intracellular domain of 4-1BB;
   the extracellular domain includes an extracellular domain of CD40L and the intracellular domain includes an intracellular domain of OX40;
   the extracellular domain includes an extracellular domain of CD40L and the intracellular domain includes an intracellular domain of CD27;
   the extracellular domain includes an extracellular domain of CD40L and the intracellular domain includes an intracellular domain of CD30;
   the extracellular domain includes an extracellular domain of CD40L and the intracellular domain includes an intracellular domain of CD40;
   the extracellular domain includes an extracellular domain of CD40L and the intracellular domain includes an intracellular domain of HVEM;
   the extracellular domain includes an extracellular domain of CD40L and the intracellular domain includes an intracellular domain of DR3;
   the extracellular domain includes an extracellular domain of CD40L and the intracellular domain includes an intracellular domain of 4-1BB;
   the extracellular domain includes an extracellular domain of FLT3L and the intracellular domain includes an intracellular domain of OX40;
   the extracellular domain includes an extracellular domain of FLT3L and the intracellular domain includes an intracellular domain of CD27;
   the extracellular domain includes an extracellular domain of FLT3L and the intracellular domain includes an intracellular domain of CD30;
   the extracellular domain includes an extracellular domain of FLT3L and the intracellular domain includes an intracellular domain of CD40;
   the extracellular domain includes an extracellular domain of FLT3L and the intracellular domain includes an intracellular domain of HVEM;
   the extracellular domain includes an extracellular domain of FLT3L and the intracellular domain includes an intracellular domain of DR3;
   the extracellular domain includes an extracellular domain of FLT3L and the intracellular domain includes an intracellular domain of 41BB;
   the extracellular domain includes an extracellular domain of LIGHT and the intracellular domain includes an intracellular domain of OX40;
   the extracellular domain includes an extracellular domain of LIGHT and the intracellular domain includes an intracellular domain of CD27;
   the extracellular domain includes an extracellular domain of LIGHT and the intracellular domain includes an intracellular domain of CD30;
   the extracellular domain includes an extracellular domain of LIGHT and the intracellular domain includes an intracellular domain of CD40;
   the extracellular domain includes an extracellular domain of LIGHT and the intracellular domain includes an intracellular domain of HVEM;
   the extracellular domain includes an extracellular domain of LIGHT and the intracellular domain includes an intracellular domain of DR3; or
   the extracellular domain includes an extracellular domain of LIGHT and the intracellular domain includes an intracellular domain of 41BB.
17. The protein of any of embodiments 2-16, wherein the intracellular domain of the different stimulatory immune cell protein is inverted.
18. The protein of embodiment 17, wherein the different stimulatory immune cell protein is 4-1BB, OX40, CD40, CD30, CD30L, CD27, DR3, HVEM, LIGHT, SLAMF1, ICOS, GITR, CD25, CD28, CD79A, CD79B, CARD11, DAP10, DAP12, FcRα, FcRβ, FcRγ, FLT3, Fyn, Lck, LAT, LRP, NKG2D, NOTCH1, NOTCH2, NOTCH3, NOTCH4, ROR2, Ryk, Slp76, pTα, TCRα, TCRβ, TIM1, TRIM, Zap70, or PTCH2.
19. The protein of embodiment 17, wherein the inverted intracellular domain is N-terminal to the extracellular domain.
20. The protein of any of embodiments 2-19, wherein the transmembrane domain includes the transmembrane domain of 4-1BB, 4-1BBL, OX40, OX40L, CD40, CD40L, CD30, CD30L, CD27, CD27L, DR3, DR3L, FLT3, FLT3L, HVEM, LIGHT, CD3ε, CD3δ, CD3ζ, CD25, CD28, CD80, CD86, CD79a, CD79aL, CD79b, CD79bL, CD84, SLAMF1, SLAMF1L, ICOS, ICOSL, GITR, GITRL, DAP10, DAP10L, DAP12, DAP12L, KIRDS2, CD2, LFA-1, Fyn, FynL, Lck, LckL, LAT, LATL, LRP, LRPL, BAFFR, SLAMF7, NKp80, NKp44, NKp30, NKp46, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, HAVCR1 (TIM1), TIM1L, TRIM, TRIML, Zap70, Zap70L, PTCH2, PTCH2L, CD122, CD132, CD226, CD160, CD19, CARD11, CARD11L, CRTAM (CD355), IL2Rβ, IL2Rγ, IL7Ra, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9 (SLAMF3), CD160, PSGL1, CD100, SLAMF6 (NTB-A, Lyl08), SLAM, BLAME (SLAMF8), SELPLG, LTBR, PAG/Cbp, NKG2D, NKG2DL, or NKG2C, FcRα, FcRαL, FcRβ, FcRβL, FcRγ, FcRγL, and CD47.
21. The protein of any of embodiments 2-20, having the sequence as set forth in SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, or SEQ ID NO: 206.
22. The protein of any of embodiments 2-21, encoded by the sequence as set forth in SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, or SEQ ID NO: 192.
23. The protein of any of embodiments 2-22, wherein the protein further includes a multimerization domain.
24. The protein of embodiment 23, wherein the multimerization domain is part of a chemically-induced dimerization system or a chemically-induced trimerization system.
25. The protein of any of embodiments 2-24, wherein the protein does not include a spacer region, a cancer antigen binding domain, and a viral antigen binding domain.
26. The protein of any of embodiments 2-25, including the extracellular domain of the stimulatory immune cell protein, the intracellular domain of the different stimulatory immune cell protein, and the transmembrane domain linking the extracellular domain to the intracellular domain.
27. A protein including an extracellular domain of an immune cell protein and a transmembrane domain, wherein the protein lacks a functional intracellular domain.
28. The protein of embodiment 27, wherein the protein is a truncated form of the immune cell protein.
29. The protein of embodiment 28, wherein the immune cell protein is 4-1BBL, OX40L, CD40L, FLT3L, or LIGHT; or CD30L, CD27L, DR3L, SLAMF1L, ICOSL, GITRL, CD80, CD86, CD79AL, CD79BL, CARD11L, DAP10L, DAP12L, FcRαL, FcRβL, FcRγL, FynL, LckL, LATL, LRPL, NKG2DL, NOTCH1L, NOTCH2L, NOTCH3L, NOTCH4L, ROR2L, RykL, Slp76L, pTαL, TIM1L, TRIML, Zap70L, or PTCH2L.

30. The protein of any of embodiments 27-29, wherein the transmembrane domain includes the transmembrane domain of 4-1BB, 4-1BBL, OX40, OX40L, CD40, CD40L, CD30, CD30L, CD27, CD27L, DR3, DR3L, FLT3, FLT3L, HVEM, LIGHT, CD3ε, CD3δ, CD3ζ, CD25, CD28, CD80, CD86, CD79a, CD79aL, CD79b, CD79bL, SLAMF1, SLAMF1L, ICOS, ICOSL, GITR, GITRL, DAP10, DAP10L, DAP12, DAP12L, KIRDS2, CD2, LFA-1, Fyn, FynL, Lck, LckL, LAT, LATL, LRP, LRPL, BAFFR, SLAMF7, NKp80, NKp44, NKp30, NKp46, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, HAVCR1 (TIM1), TIM1L, TRIM, TRIML, Zap70, Zap70L, PTCH2, PTCH2L, CD122, CD132, CD226, CD160, CD19, CARD11, CARD11L, CRTAM (CD355), IL2Rβ, IL2Rγ, IL7Ra, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9 (SLAMF3), CD160, PSGL1, CD100, SLAMF6 (NTB-A, Lyl08), SLAM, BLAME (SLAMF8), SELPLG, LTBR, PAG/Cbp, NKG2D, NKG2DL, or NKG2C, FcRα, FcRαL, FcRβ, FcRβL, FcRγ, FcRγL, or CD47.
31. The protein of any of embodiments 27-30 having the sequence as set forth in SEQ ID NO: 143, SEQ ID NO: 144, SEQ ID NO: 145, SEQ ID NO: 147, SEQ ID NO: 149, or SEQ ID NO: 150.
32. The protein of any of embodiments 27-31, encoded by the sequence as set forth in SEQ ID NO: 146 or SEQ ID NO: 148.
33. A protein including an inverted intracellular signaling domain of a Type I protein and an extracellular domain of a Type II protein, wherein the inverted intracellular signaling domain is N-terminal to the extracellular domain and wherein the inverted intracellular signaling domain and the extracellular domain are linked through a transmembrane domain.
34. The protein of embodiment 33, wherein the extracellular domain is not inverted.
35. The protein of embodiments 33 or 34, wherein the Type I protein is a co-stimulatory protein.
36. The protein of any of embodiments 33-35, wherein the Type II protein is a co-stimulatory protein ligand.
37. The protein of embodiment 35, wherein the co-stimulatory protein is 4-1BB, OX40, CD40, CD30, CD30L, CD27, DR3, HVEM, SLAMF1, ICOS, GITR, CD25, CD28, CD79A, CD79B, CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TIM1, TRIM, Zap70, or PTCH2.
38. The protein of embodiments 35 or 37, wherein the co-stimulatory protein is 4-1BB, OX40, CD40, CD30, CD27, DR3, or HVEM.
39. The protein of embodiment 36, wherein the co-stimulatory protein ligand is 4-1BBL, OX40L, CD40L, CD30L, CD27L, DR3L, LIGHT, SLAMF1L, ICOSL, GITRL, CD80, CD86, CD79AL, CD79BL, CARD11L, DAP10L, DAP12L, FcRαL, FcRβL, FcRγL, FLT3L, FynL, LckL, LATL, LRPL, NKG2DL, NOTCH1L, NOTCH2L, NOTCH3L, NOTCH4L, ROR2L, RykL, Slp76L, pTαL, TIM1L, TRIML, Zap70L, or PTCH2L
40. The protein of embodiments 36 or 39, wherein the co-stimulatory protein ligand is 4-1BBL, OX40L, CD40L, CD30L, CD27L, DR3L, FLT3L, or LIGHT.
41. The protein of any of embodiments 2-26, 27-32, or 33-41 further including a signal peptide.
42. A nucleotide including a gene encoding a protein of any of embodiments 2-26, 27-32, or 33-41.
43. The nucleotide of embodiment 42, wherein the nucleotide includes regulatory elements including an inducible promoter.
44. A cell genetically modified to express a protein of any of embodiments 2-26, 27-32, or 33-41.
45. The cell of embodiment 44, wherein the cell is an immune cell.
46. The cell of embodiment 45, wherein the immune cell is a T cell or a natural killer (NK) cell.
47. The cell of embodiment 46, wherein the T cell is a CD4+ or a CD8+ T cell.
48. The cell of embodiment 45, wherein the immune cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MAIT) cell, a B cell, a dendritic cell, a monocyte or a macrophage.
49. A method of genetically-modifying an immune cell to express a protein of any of embodiments 2-26, 27-32, or 33-41 including introducing a nucleotide of embodiments 42 or 43 into the immune cell.
50. A formulation including an immune cell of embodiment 45 and a pharmaceutically acceptable carrier.
51. A nanoparticle including a nucleotide of embodiments 42 or 43.
52. The nanoparticle of embodiment 51, further including a cell targeting ligand.
53. The nanoparticle of embodiment 52, wherein the cell targeting ligand binds a T cell surface marker.
54. The nanoparticle of embodiment 53, wherein the T cell surface marker is CD3, CD4, or CD8.
55. The nanoparticle of embodiment 52, wherein the cell targeting ligand binds an iPSC marker, a TIL marker, a MIL marker, an NKT marker, a MATT cell marker, a B cell marker, a dendritic cell marker, a monocyte marker, or a macrophage marker.
56. A composition including a nanoparticle of any of embodiments 51-55 and a pharmaceutically acceptable carrier.
57. A method of modulating an immune response in a subject in need thereof including administering a therapeutically effective amount of a nucleotide of embodiment 42 or 43, a formulation of embodiment 50, or a nanoparticle of any of embodiments 51-55 to the subject, thereby modulating the immune response in the subject thereof.
58. The method of embodiment 57, wherein the modulating is stimulating.
59. The method of embodiments 57 or 58, wherein the modulating increases stimulatory intracellular signaling in immune cells within the subject.
60. The method of embodiment 59, wherein the increased stimulatory intracellular signaling in immune cells within the subject results in increased immune cell proliferation and/or increased immune cell activation in the subject, as compared to a comparable subject who was not administered the therapeutically effective amount.
61. The method of any of embodiments 57-60, wherein the modulating promotes an anti-cancer effect or an anti-infection effect in the subject.

(vii) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gin), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleotide, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleotide, or gene sequences disclosed herein.

“% sequence identity” refers to a relationship between two or more sequences, as determined by comparing the sequences. In the art, “identity” also means the degree of sequence relatedness between protein, nucleotide, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A protein consisting of
an extracellular domain of OX40L;
an intracellular domain of CD30, HVEM, or CD40; and
a transmembrane domain linking the extracellular domain to the intracellular domain.

2. A protein comprising an extracellular domain of a stimulatory immune cell protein, an intracellular domain of a different stimulatory immune cell protein, and a transmembrane domain linking the extracellular domain to the intracellular domain.

3. The protein of claim 2, wherein expression of the protein by an immune cell increases activation of the immune cell in the presence of a ligand, when compared to an immune cell that does not express the protein in the presence of the same ligand.

4. The protein of claim 2, wherein the extracellular domain is an extracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.

5. The protein of claim 2, wherein the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.

6. The protein of claim 2, wherein
the extracellular domain is an extracellular domain of 4-1BB and the intracellular domain is an intracellular domain of 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.
the extracellular domain is an extracellular domain of 4-1BBL and the intracellular domain is an intracellular domain of 4-1BB, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of OX40 and the intracellular domain is an intracellular domain of 4-1BB, 4-1BBL, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of OX40L and the intracellular domain is an intracellular domain of 4-1BB, 4-1BBL, OX40, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD40 and the intracellular domain is an intracellular domain of 4-1BB, 4-1BBL, OX40, OX40L, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD40L and the intracellular domain is an intracellular domain of 4-1BB, 4-1BBL, OX40, OX40L, CD40, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD30 and the intracellular domain is an intracellular domain of 4-1BB, 4-1BBL, OX40, OX40L, CD40, CD40L, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD30L and the intracellular domain is an intracellular domain of 4-1BB, 4-1BBL, OX40, OX40L, CD40, CD40L, CD30, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD27 and the intracellular domain is an intracellular domain of 4-1BB, 4-1BBL, OX40, OX40L, CD40, CD40L, CD30, CD30L, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of CD27L and the intracellular domain is an intracellular domain of 4-1BB, 4-1BBL, OX40, OX40L, CD40, CD40L, CD30, CD30L, CD27, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of DR3 and the intracellular domain is an intracellular domain of 4-1BB, 4-1BBL, OX40, OX40L, CD40, CD40L, CD30, CD30L, CD27, CD27L, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of DR3L and the intracellular domain is an intracellular domain of 4-1BB, 4-1BBL, OX40, OX40L, CD40, CD40L, CD30, CD30L, CD27, CD27L, DR3, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of FLT3L and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L;
the extracellular domain is an extracellular domain of HVEM and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L; or
the extracellular domain is an extracellular domain of LIGHT and the intracellular domain is an intracellular domain of 4-1BB, 4-1BBL, OX40, OX40L, CD40, CD40L, CD30, CD30L, CD27, CD27L, DR3, DR3L, HVEM, LIGHT, SLAMF1, SLAMF1L, ICOS, ICOSL, GITR, GITRL, CD25, CD28, CD80, CD86, CD79A, CD79AL, CD79B, CD79BL, CARD11, CARD11 L, DAP10, DAP10L, DAP12, DAP12L, FcRα, FcRαL, FcRβ, FcRβL, FcRγ, FcRγL, FLT3, FLT3L, Fyn, FynL, Lck, LckL, LAT, LATL, LRP, LRPL, BLAME, CD122, CD132, CD2, CD226, CD3ε, CD3δ, CD3ζ, CD84, CRTAM (CD355), FLT3L, TIM1, Ly9, SLAMF7, NKG2D, NKG2DL, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, TIM1, TIM1L, TRIM, TRIML, Zap70, Zap70L, PTCH2, or PTCH2L.

7. The protein of claim 2, further comprising at least a second copy of the intracellular domain, the second copy also linked to the extracellular domain through the transmembrane domain.

8. The protein of claim 2, further comprising the intracellular domain of the stimulatory immune cell protein.

9. The protein of claim 8, wherein the stimulatory immune cell protein comprises 4-1BB, CD40, or OX40.

10. The protein of claim 2, further comprising an intracellular domain of a third stimulatory immune cell protein.

11. The protein of claim 10, wherein the third stimulatory immune cell protein comprises 4-1BB, CD40, or OX40.

12. The protein of claim 2, wherein the stimulatory immune cell protein and the different immune stimulatory protein both comprise dimerizing intracellular domains.

13. The protein of claim 2, wherein
the extracellular domain comprises an extracellular domain of CD2 and the intracellular domain comprises an intracellular domain of CD28;
the extracellular domain comprises an extracellular domain of CD2 and the intracellular domain comprises an intracellular domain of CD226;
the extracellular domain comprises an extracellular domain of CD2 and the intracellular domain comprises an intracellular domain of CRTAM;
the extracellular domain comprises an extracellular domain of CD2 and the intracellular domain comprises an intracellular domain of TIM1;
the extracellular domain comprises an extracellular domain of CD2 and the intracellular domain comprises an intracellular domain of ICOS;
the extracellular domain comprises an extracellular domain of CD2 and the intracellular domain comprises an intracellular domain of SLAMF1;
the extracellular domain comprises an extracellular domain of CD2 and the intracellular domain comprises an intracellular domain of Ly9;
the extracellular domain comprises an extracellular domain of CD2 and the intracellular domain comprises an intracellular domain of CD84;
the extracellular domain comprises an extracellular domain of CD2 and the intracellular domain comprises an intracellular domain of SLAMF7;
the extracellular domain comprises an extracellular domain of CD2 and the intracellular domain comprises an intracellular domain of BLAME;
the extracellular domain comprises an extracellular domain of CD28 and the intracellular domain comprises an intracellular domain of CD2;
the extracellular domain comprises an extracellular domain of CD28 and the intracellular domain comprises an intracellular domain of CD226;
the extracellular domain comprises an extracellular domain of CD28 and the intracellular domain comprises an intracellular domain of CRTAM;
the extracellular domain comprises an extracellular domain of CD28 and the intracellular domain comprises an intracellular domain of TIM1;
the extracellular domain comprises an extracellular domain of CD28 and the intracellular domain comprises an intracellular domain of ICOS;
the extracellular domain comprises an extracellular domain of CD28 and the intracellular domain comprises an intracellular domain of SLAMF1;
the extracellular domain comprises an extracellular domain of CD28 and the intracellular domain comprises an intracellular domain of Ly9;
the extracellular domain comprises an extracellular domain of CD28 and the intracellular domain comprises an intracellular domain of CD84;
the extracellular domain comprises an extracellular domain of CD28 and the intracellular domain comprises an intracellular domain of SLAMF7;
the extracellular domain comprises an extracellular domain of CD28 and the intracellular domain comprises an intracellular domain of BLAME;
the extracellular domain comprises an extracellular domain of CD226 and the intracellular domain comprises an intracellular domain of CD2;
the extracellular domain comprises an extracellular domain of CD226 and the intracellular domain comprises an intracellular domain of CD28;
the extracellular domain comprises an extracellular domain of CD226 and the intracellular domain comprises an intracellular domain of CRTAM;
the extracellular domain comprises an extracellular domain of CD226 and the intracellular domain comprises an intracellular domain of TIM1;
the extracellular domain comprises an extracellular domain of CD226 and the intracellular domain comprises an intracellular domain of ICOS;
the extracellular domain comprises an extracellular domain of CD226 and the intracellular domain comprises an intracellular domain of SLAMF1;
the extracellular domain comprises an extracellular domain of CD226 and the intracellular domain comprises an intracellular domain of Ly9;
the extracellular domain comprises an extracellular domain of CD226 and the intracellular domain comprises an intracellular domain of CD84;
the extracellular domain comprises an extracellular domain of CD226 and the intracellular domain comprises an intracellular domain of SLAMF7;
the extracellular domain comprises an extracellular domain of CD226 and the intracellular domain comprises an intracellular domain of BLAME;
the extracellular domain comprises an extracellular domain of CRTAM and the intracellular domain comprises an intracellular domain of CD2;
the extracellular domain comprises an extracellular domain of CRTAM and the intracellular domain comprises an intracellular domain of CD28;
the extracellular domain comprises an extracellular domain of CRTAM and the intracellular domain comprises an intracellular domain of CD226;
the extracellular domain comprises an extracellular domain of CRTAM and the intracellular domain comprises an intracellular domain of TIM1;
the extracellular domain comprises an extracellular domain of CRTAM and the intracellular domain comprises an intracellular domain of ICOS;
the extracellular domain comprises an extracellular domain of CRTAM and the intracellular domain comprises an intracellular domain of SLAMF1;
the extracellular domain comprises an extracellular domain of CRTAM and the intracellular domain comprises an intracellular domain of Ly9;
the extracellular domain comprises an extracellular domain of CRTAM and the intracellular domain comprises an intracellular domain of CD84;
the extracellular domain comprises an extracellular domain of CRTAM and the intracellular domain comprises an intracellular domain of SLAMF7;
the extracellular domain comprises an extracellular domain of CRTAM and the intracellular domain comprises an intracellular domain of BLAME;
the extracellular domain comprises an extracellular domain of TIM1 and the intracellular domain comprises an intracellular domain of CD2;
the extracellular domain comprises an extracellular domain of TIM1 and the intracellular domain comprises an intracellular domain of CD28;
the extracellular domain comprises an extracellular domain of TIM1 and the intracellular domain comprises an intracellular domain of CD226;
the extracellular domain comprises an extracellular domain of TIM1 and the intracellular domain comprises an intracellular domain of CRTAM;
the extracellular domain comprises an extracellular domain of TIM1 and the intracellular domain comprises an intracellular domain of ICOS;
the extracellular domain comprises an extracellular domain of TIM1 and the intracellular domain comprises an intracellular domain of SLAMF1;
the extracellular domain comprises an extracellular domain of TIM1 and the intracellular domain comprises an intracellular domain of Ly9;
the extracellular domain comprises an extracellular domain of TIM1 and the intracellular domain comprises an intracellular domain of CD84;
the extracellular domain comprises an extracellular domain of TIM1 and the intracellular domain comprises an intracellular domain of SLAMF7;
the extracellular domain comprises an extracellular domain of TIM1 and the intracellular domain comprises an intracellular domain of BLAME;
the extracellular domain comprises an extracellular domain of ICOS and the intracellular domain comprises an intracellular domain of CD2;
the extracellular domain comprises an extracellular domain of ICOS and the intracellular domain comprises an intracellular domain of CD28;
the extracellular domain comprises an extracellular domain of ICOS and the intracellular domain comprises an intracellular domain of CD226;
the extracellular domain comprises an extracellular domain of ICOS and the intracellular domain comprises an intracellular domain of CRTAM;
the extracellular domain comprises an extracellular domain of ICOS and the intracellular domain comprises an intracellular domain of TIM1;
the extracellular domain comprises an extracellular domain of ICOS and the intracellular domain comprises an intracellular domain of SLAMF1;
the extracellular domain comprises an extracellular domain of ICOS and the intracellular domain comprises an intracellular domain of Ly9;
the extracellular domain comprises an extracellular domain of ICOS and the intracellular domain comprises an intracellular domain of CD84;
the extracellular domain comprises an extracellular domain of ICOS and the intracellular domain comprises an intracellular domain of SLAMF7;
the extracellular domain comprises an extracellular domain of ICOS and the intracellular domain comprises an intracellular domain of BLAME;
the extracellular domain comprises an extracellular domain of SLAMF1 and the intracellular domain comprises an intracellular domain of CD2;
the extracellular domain comprises an extracellular domain of SLAMF1 and the intracellular domain comprises an intracellular domain of CD28;
the extracellular domain comprises an extracellular domain of SLAMF1 and the intracellular domain comprises an intracellular domain of CD226;
the extracellular domain comprises an extracellular domain of SLAMF1 and the intracellular domain comprises an intracellular domain of CRTAM;
the extracellular domain comprises an extracellular domain of SLAMF1 and the intracellular domain comprises an intracellular domain of TIM1;
the extracellular domain comprises an extracellular domain of SLAMF1 and the intracellular domain comprises an intracellular domain of ICOS;
the extracellular domain comprises an extracellular domain of SLAMF1 and the intracellular domain comprises an intracellular domain of Ly9;
the extracellular domain comprises an extracellular domain of SLAMF1 and the intracellular domain comprises an intracellular domain of CD84;
the extracellular domain comprises an extracellular domain of SLAMF1 and the intracellular domain comprises an intracellular domain of SLAMF7;
the extracellular domain comprises an extracellular domain of SLAMF1 and the intracellular domain comprises an intracellular domain of BLAME;
the extracellular domain comprises an extracellular domain of Ly9 and the intracellular domain comprises an intracellular domain of CD2;
the extracellular domain comprises an extracellular domain of Ly9 and the intracellular domain comprises an intracellular domain of CD28;
the extracellular domain comprises an extracellular domain of Ly9 and the intracellular domain comprises an intracellular domain of CD226;
the extracellular domain comprises an extracellular domain of Ly9 and the intracellular domain comprises an intracellular domain of CRTAM;
the extracellular domain comprises an extracellular domain of Ly9 and the intracellular domain comprises an intracellular domain of TIM1;
the extracellular domain comprises an extracellular domain of Ly9 and the intracellular domain comprises an intracellular domain of ICOS;
the extracellular domain comprises an extracellular domain of Ly9 and the intracellular domain comprises an intracellular domain of SLAMF1;
the extracellular domain comprises an extracellular domain of Ly9 and the intracellular domain comprises an intracellular domain of CD84;
the extracellular domain comprises an extracellular domain of Ly9 and the intracellular domain comprises an intracellular domain of SLAMF7;
the extracellular domain comprises an extracellular domain of Ly9 and the intracellular domain comprises an intracellular domain of BLAME;
the extracellular domain comprises an extracellular domain of CD84 and the intracellular domain comprises an intracellular domain of CD2;
the extracellular domain comprises an extracellular domain of CD84 and the intracellular domain comprises an intracellular domain of CD28;
the extracellular domain comprises an extracellular domain of CD84 and the intracellular domain comprises an intracellular domain of CD226;
the extracellular domain comprises an extracellular domain of CD84 and the intracellular domain comprises an intracellular domain of CRTAM;
the extracellular domain comprises an extracellular domain of CD84 and the intracellular domain comprises an intracellular domain of TIM1;
the extracellular domain comprises an extracellular domain of CD84 and the intracellular domain comprises an intracellular domain of ICOS;
the extracellular domain comprises an extracellular domain of CD84 and the intracellular domain comprises an intracellular domain of SLAMF1;
the extracellular domain comprises an extracellular domain of CD84 and the intracellular domain comprises an intracellular domain of Ly9;
the extracellular domain comprises an extracellular domain of CD84 and the intracellular domain comprises an intracellular domain of SLAMF7;
the extracellular domain comprises an extracellular domain of CD84 and the intracellular domain comprises an intracellular domain of BLAME;
the extracellular domain comprises an extracellular domain of SLAMF7 and the intracellular domain comprises an intracellular domain of CD2;
the extracellular domain comprises an extracellular domain of SLAMF7 and the intracellular domain comprises an intracellular domain of CD28;
the extracellular domain comprises an extracellular domain of SLAMF7 and the intracellular domain comprises an intracellular domain of CD226;
the extracellular domain comprises an extracellular domain of SLAMF7 and the intracellular domain comprises an intracellular domain of CRTAM;
the extracellular domain comprises an extracellular domain of SLAMF7 and the intracellular domain comprises an intracellular domain of TIM1;
the extracellular domain comprises an extracellular domain of SLAMF7 and the intracellular domain comprises an intracellular domain of ICOS;
the extracellular domain comprises an extracellular domain of SLAMF7 and the intracellular domain comprises an intracellular domain of SLAMF1;
the extracellular domain comprises an extracellular domain of SLAMF7 and the intracellular domain comprises an intracellular domain of Ly9;
the extracellular domain comprises an extracellular domain of SLAMF7 and the intracellular domain comprises an intracellular domain of CD84;
the extracellular domain comprises an extracellular domain of SLAMF7 and the intracellular domain comprises an intracellular domain of BLAME;
the extracellular domain comprises an extracellular domain of BLAME and the intracellular domain comprises an intracellular domain of CD2;
the extracellular domain comprises an extracellular domain of BLAME and the intracellular domain comprises an intracellular domain of CD28;
the extracellular domain comprises an extracellular domain of BLAME and the intracellular domain comprises an intracellular domain of CD226;
the extracellular domain comprises an extracellular domain of BLAME and the intracellular domain comprises an intracellular domain of CRTAM;
the extracellular domain comprises an extracellular domain of BLAME and the intracellular domain comprises an intracellular domain of TIM1;
the extracellular domain comprises an extracellular domain of BLAME and the intracellular domain comprises an intracellular domain of ICOS;
the extracellular domain comprises an extracellular domain of BLAME and the intracellular domain comprises an intracellular domain of SLAMF1;
the extracellular domain comprises an extracellular domain of BLAME and the intracellular domain comprises an intracellular domain of Ly9;
the extracellular domain comprises an extracellular domain of BLAME and the intracellular domain comprises an intracellular domain of CD84; or
the extracellular domain comprises an extracellular domain of BLAME and the intracellular domain comprises an intracellular domain of SLAMF7.

14. The protein of claim 2, wherein the stimulatory immune cell protein and the different immune stimulatory protein both comprise trimerizing intracellular domains.

15. The protein of claim 2, wherein
the extracellular domain comprises an extracellular domain of OX40 and the intracellular domain comprises an intracellular domain of CD27;
the extracellular domain comprises an extracellular domain of OX40 and the intracellular domain comprises an intracellular domain of CD30;
the extracellular domain comprises an extracellular domain of OX40 and the intracellular domain comprises an intracellular domain of CD40;
the extracellular domain comprises an extracellular domain of OX40 and the intracellular domain comprises an intracellular domain of HVEM;
the extracellular domain comprises an extracellular domain of OX40 and the intracellular domain comprises an intracellular domain of DR3;
the extracellular domain comprises an extracellular domain of OX40 and the intracellular domain comprises an intracellular domain of 41BB;
the extracellular domain comprises an extracellular domain of OX40 and the intracellular domain comprises an intracellular domain of GITR;
the extracellular domain comprises an extracellular domain of OX40 and the intracellular domain comprises an intracellular domain of LIGHT;
the extracellular domain comprises an extracellular domain of CD27 and the intracellular domain comprises an intracellular domain of OX40;
the extracellular domain comprises an extracellular domain of CD27 and the intracellular domain comprises an intracellular domain of CD30;
the extracellular domain comprises an extracellular domain of CD27 and the intracellular domain comprises an intracellular domain of CD40;
the extracellular domain comprises an extracellular domain of CD27 and the intracellular domain comprises an intracellular domain of HVEM;
the extracellular domain comprises an extracellular domain of CD27 and the intracellular domain comprises an intracellular domain of DR3;
the extracellular domain comprises an extracellular domain of CD27 and the intracellular domain comprises an intracellular domain of 41BB;
the extracellular domain comprises an extracellular domain of CD27 and the intracellular domain comprises an intracellular domain of GITR;
the extracellular domain comprises an extracellular domain of CD27 and the intracellular domain comprises an intracellular domain of LIGHT;
the extracellular domain comprises an extracellular domain of CD30 and the intracellular domain comprises an intracellular domain of OX40;
the extracellular domain comprises an extracellular domain of CD30 and the intracellular domain comprises an intracellular domain of CD27;
the extracellular domain comprises an extracellular domain of CD30 and the intracellular domain comprises an intracellular domain of CD40;
the extracellular domain comprises an extracellular domain of CD30 and the intracellular domain comprises an intracellular domain of HVEM;
the extracellular domain comprises an extracellular domain of CD30 and the intracellular domain comprises an intracellular domain of DR3;
the extracellular domain comprises an extracellular domain of CD30 and the intracellular domain comprises an intracellular domain of 41BB;
the extracellular domain comprises an extracellular domain of CD30 and the intracellular domain comprises an intracellular domain of GITR;
the extracellular domain comprises an extracellular domain of CD30 and the intracellular domain comprises an intracellular domain of LIGHT;
the extracellular domain comprises an extracellular domain of CD40 and the intracellular domain comprises an intracellular domain of OX40;
the extracellular domain comprises an extracellular domain of CD40 and the intracellular domain comprises an intracellular domain of CD27;
the extracellular domain comprises an extracellular domain of CD40 and the intracellular domain comprises an intracellular domain of CD30;
the extracellular domain comprises an extracellular domain of CD40 and the intracellular domain comprises an intracellular domain of HVEM;
the extracellular domain comprises an extracellular domain of CD40 and the intracellular domain comprises an intracellular domain of DR3;
the extracellular domain comprises an extracellular domain of CD40 and the intracellular domain comprises an intracellular domain of 41BB;
the extracellular domain comprises an extracellular domain of CD40 and the intracellular domain comprises an intracellular domain of GITR;
the extracellular domain comprises an extracellular domain of CD40 and the intracellular domain comprises an intracellular domain of LIGHT;
the extracellular domain comprises an extracellular domain of HVEM and the intracellular domain comprises an intracellular domain of OX40;
the extracellular domain comprises an extracellular domain of HVEM and the intracellular domain comprises an intracellular domain of CD27;
the extracellular domain comprises an extracellular domain of HVEM and the intracellular domain comprises an intracellular domain of CD30;
the extracellular domain comprises an extracellular domain of HVEM and the intracellular domain comprises an intracellular domain of CD40;
the extracellular domain comprises an extracellular domain of HVEM and the intracellular domain comprises an intracellular domain of DR3;
the extracellular domain comprises an extracellular domain of HVEM and the intracellular domain comprises an intracellular domain of 41BB;
the extracellular domain comprises an extracellular domain of HVEM and the intracellular domain comprises an intracellular domain of GITR;
the extracellular domain comprises an extracellular domain of HVEM and the intracellular domain comprises an intracellular domain of LIGHT;
the extracellular domain comprises an extracellular domain of DR3 and the intracellular domain comprises an intracellular domain of OX40;
the extracellular domain comprises an extracellular domain of DR3 and the intracellular domain comprises an intracellular domain of CD27;
the extracellular domain comprises an extracellular domain of DR3 and the intracellular domain comprises an intracellular domain of CD30;
the extracellular domain comprises an extracellular domain of DR3 and the intracellular domain comprises an intracellular domain of CD40;
the extracellular domain comprises an extracellular domain of DR3 and the intracellular domain comprises an intracellular domain of HVEM;
the extracellular domain comprises an extracellular domain of DR3 and the intracellular domain comprises an intracellular domain of 41BB;
the extracellular domain comprises an extracellular domain of DR3 and the intracellular domain comprises an intracellular domain of GITR;
the extracellular domain comprises an extracellular domain of DR3 and the intracellular domain comprises an intracellular domain of LIGHT;
the extracellular domain comprises an extracellular domain of 41BB and the intracellular domain comprises an intracellular domain of OX40;
the extracellular domain comprises an extracellular domain of 41BB and the intracellular domain comprises an intracellular domain of CD27;
the extracellular domain comprises an extracellular domain of 41BB and the intracellular domain comprises an intracellular domain of CD30;
the extracellular domain comprises an extracellular domain of 41BB and the intracellular domain comprises an intracellular domain of CD40;
the extracellular domain comprises an extracellular domain of 41BB and the intracellular domain comprises an intracellular domain of HVEM;
the extracellular domain comprises an extracellular domain of 41BB and the intracellular domain comprises an intracellular domain of DR3;
the extracellular domain comprises an extracellular domain of 41BB and the intracellular domain comprises an intracellular domain of GITR;
the extracellular domain comprises an extracellular domain of 41BB and the intracellular domain comprises an intracellular domain of LIGHT;
the extracellular domain comprises an extracellular domain of GITR and the intracellular domain comprises an intracellular domain of OX40;
the extracellular domain comprises an extracellular domain of GITR and the intracellular domain comprises an intracellular domain of CD27;
the extracellular domain comprises an extracellular domain of GITR and the intracellular domain comprises an intracellular domain of CD30;
the extracellular domain comprises an extracellular domain of GITR and the intracellular domain comprises an intracellular domain of CD40;
the extracellular domain comprises an extracellular domain of GITR and the intracellular domain comprises an intracellular domain of HVEM;
the extracellular domain comprises an extracellular domain of GITR and the intracellular domain comprises an intracellular domain of DR3;
the extracellular domain comprises an extracellular domain of GITR and the intracellular domain comprises an intracellular domain of 41BB;
the extracellular domain comprises an extracellular domain of GITR and the intracellular domain comprises an intracellular domain of LIGHT;
the extracellular domain comprises an extracellular domain of LIGHT and the intracellular domain comprises an intracellular domain of OX40;
the extracellular domain comprises an extracellular domain of LIGHT and the intracellular domain comprises an intracellular domain of CD27;
the extracellular domain comprises an extracellular domain of LIGHT and the intracellular domain comprises an intracellular domain of CD30;
the extracellular domain comprises an extracellular domain of LIGHT and the intracellular domain comprises an intracellular domain of CD40;
the extracellular domain comprises an extracellular domain of LIGHT and the intracellular domain comprises an intracellular domain of HVEM;
the extracellular domain comprises an extracellular domain of LIGHT and the intracellular domain comprises an intracellular domain of DR3;
the extracellular domain comprises an extracellular domain of LIGHT and the intracellular domain comprises an intracellular domain of 41BB; or
the extracellular domain comprises an extracellular domain of LIGHT and the intracellular domain comprises an intracellular domain of GITR.

16. The protein of claim 2, wherein:
the extracellular domain comprises an extracellular domain of 4-1BBL and the intracellular domain comprises an intracellular domain of OX40;
the extracellular domain comprises an extracellular domain of 4-1BBL and the intracellular domain comprises an intracellular domain of CD27;
the extracellular domain comprises an extracellular domain of 4-1BBL and the intracellular domain comprises an intracellular domain of CD30;
the extracellular domain comprises an extracellular domain of 4-1BBL and the intracellular domain comprises an intracellular domain of CD40;
the extracellular domain comprises an extracellular domain of 4-1BBL and the intracellular domain comprises an intracellular domain of HVEM;
the extracellular domain comprises an extracellular domain of 4-1BBL and the intracellular domain comprises an intracellular domain of DR3;
the extracellular domain comprises an extracellular domain of 4-1BBL and the intracellular domain comprises an intracellular domain of 4-1BB;
the extracellular domain comprises an extracellular domain of OX40L and the intracellular domain comprises an intracellular domain of OX40;
the extracellular domain comprises an extracellular domain of OX40L and the intracellular domain comprises an intracellular domain of CD27;
the extracellular domain comprises an extracellular domain of OX40L and the intracellular domain comprises an intracellular domain of CD30;
the extracellular domain comprises an extracellular domain of OX40L and the intracellular domain comprises an intracellular domain of CD40;
the extracellular domain comprises an extracellular domain of OX40L and the intracellular domain comprises an intracellular domain of HVEM;
the extracellular domain comprises an extracellular domain of OX40L and the intracellular domain comprises an intracellular domain of DR3;
the extracellular domain comprises an extracellular domain of OX40L and the intracellular domain comprises an intracellular domain of 4-1BB;
the extracellular domain comprises an extracellular domain of CD40L and the intracellular domain comprises an intracellular domain of OX40;
the extracellular domain comprises an extracellular domain of CD40L and the intracellular domain comprises an intracellular domain of CD27;
the extracellular domain comprises an extracellular domain of CD40L and the intracellular domain comprises an intracellular domain of CD30;
the extracellular domain comprises an extracellular domain of CD40L and the intracellular domain comprises an intracellular domain of CD40;
the extracellular domain comprises an extracellular domain of CD40L and the intracellular domain comprises an intracellular domain of HVEM;
the extracellular domain comprises an extracellular domain of CD40L and the intracellular domain comprises an intracellular domain of DR3;
the extracellular domain comprises an extracellular domain of CD40L and the intracellular domain comprises an intracellular domain of 4-1BB;
the extracellular domain comprises an extracellular domain of FLT3L and the intracellular domain comprises an intracellular domain of OX40;
the extracellular domain comprises an extracellular domain of FLT3L and the intracellular domain comprises an intracellular domain of CD27;
the extracellular domain comprises an extracellular domain of FLT3L and the intracellular domain comprises an intracellular domain of CD30;
the extracellular domain comprises an extracellular domain of FLT3L and the intracellular domain comprises an intracellular domain of CD40;
the extracellular domain comprises an extracellular domain of FLT3L and the intracellular domain comprises an intracellular domain of HVEM;
the extracellular domain comprises an extracellular domain of FLT3L and the intracellular domain comprises an intracellular domain of DR3;
the extracellular domain comprises an extracellular domain of FLT3L and the intracellular domain comprises an intracellular domain of 41BB;
the extracellular domain comprises an extracellular domain of LIGHT and the intracellular domain comprises an intracellular domain of OX40;
the extracellular domain comprises an extracellular domain of LIGHT and the intracellular domain comprises an intracellular domain of CD27;
the extracellular domain comprises an extracellular domain of LIGHT and the intracellular domain comprises an intracellular domain of CD30;
the extracellular domain comprises an extracellular domain of LIGHT and the intracellular domain comprises an intracellular domain of CD40;
the extracellular domain comprises an extracellular domain of LIGHT and the intracellular domain comprises an intracellular domain of HVEM;
the extracellular domain comprises an extracellular domain of LIGHT and the intracellular domain comprises an intracellular domain of DR3; or
the extracellular domain comprises an extracellular domain of LIGHT and the intracellular domain comprises an intracellular domain of 41BB.

17. The protein of any of claims 2, wherein the intracellular domain of the different stimulatory immune cell protein is inverted.

18. The protein of claim 17, wherein the different stimulatory immune cell protein is 4-1BB, OX40, CD40, CD30, CD30L, CD27, DR3, HVEM, LIGHT, SLAMF1, ICOS, GITR, CD25, CD28, CD79A, CD79B, CARD11, DAP10, DAP12, FcRα, FcRβ, FcRγ, FLT3, Fyn, Lck, LAT, LRP, NKG2D, NOTCH1, NOTCH2, NOTCH3, NOTCH4, ROR2, Ryk, Slp76, pTα, TCRα, TCRβ, TIM1, TRIM, Zap70, or PTCH2.

19. The protein of claim 17, wherein the inverted intracellular domain is N-terminal to the extracellular domain.

20. The protein of claim 2, wherein the transmembrane domain comprises the transmembrane domain of 4-1BB, 4-1BBL, OX40, OX40L, CD40, CD40L, CD30, CD30L, CD27, CD27L, DR3, DR3L, FLT3, FLT3L, HVEM, LIGHT, CD3ε, CD3δ, CD3ζ, CD25, CD28, CD80, CD86, CD79a, CD79aL, CD79b, CD79bL, CD84, SLAMF1, SLAMF1L, ICOS, ICOSL, GITR, GITRL, DAP10, DAP10L, DAP12, DAP12L, KIRDS2, CD2, LFA-1, Fyn, FynL, Lck, LckL, LAT, LATL, LRP, LRPL, BAFFR, SLAMF7, NKp80, NKp44, NKp30, NKp46, NOTCH1, NOTCH1L, NOTCH2, NOTCH2L, NOTCH3, NOTCH3L, NOTCH4, NOTCH4L, ROR2, ROR2L, Ryk, RykL, SIp76, SIp76L, pTα, pTαL, TCRα, TCRβ, HAVCR1 (TIM1), TIM1L, TRIM, TRIML, Zap70, Zap70L, PTCH2, PTCH2L, CD122, CD132, CD226, CD160, CD19, CARD11, CARD11L, CRTAM (CD355), IL2Rβ, IL2Rγ, IL7Ra, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9 (SLAMF3), CD160, PSGL1, CD100, SLAMF6 (NTB-A, Lyl08), SLAM, BLAME (SLAMF8), SELPLG, LTBR, PAG/Cbp, NKG2D, NKG2DL, or NKG2C, FcRα, FcRαL, FcRβ, FcRβL, FcRγ, FcRγL, and CD47.

21. The protein of claim 2, having the sequence as set forth in SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, or SEQ ID NO: 206.

22. The protein of claim 2, encoded by the sequence as set forth in SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, or SEQ ID NO: 192.

23. The protein of claim 2, wherein the protein further comprises a multimerization domain.

24. The protein of claim 23, wherein the multimerization domain is part of a chemically-induced dimerization system or a chemically-induced trimerization system.

25. The protein of claim 2, wherein the protein does not comprise a spacer region, a cancer antigen binding domain, and a viral antigen binding domain.

26. The protein of claim 2, consisting of or consisting essentially of the extracellular domain of the stimulatory immune cell protein, the intracellular domain of the different stimulatory immune cell protein, and the transmembrane domain linking the extracellular domain to the intracellular domain.

27. A protein comprising an extracellular domain of an immune cell protein and a transmembrane domain, wherein the protein lacks a functional intracellular domain.

28. The protein of claim 27, wherein the protein is a truncated form of the immune cell protein.

29. The protein of claim 28, wherein the immune cell protein is 4-1BBL, OX40L, CD40L, FLT3, or LIGHT.

30. The protein of claim 27, wherein the transmembrane domain comprises the transmembrane domain of 4-1BB, 4-1BBL, OX40, OX40L, CD40, CD40L, CD30, CD30L, CD27, CD27L, DR3, DR3L, FLT3, FLT3L, HVEM, LIGHT, CD3ε, CD3δ, CD3ζ, CD25, CD28, CD80, CD86, CD79a, CD79aL, CD79b, CD79bL, SLAMF1, SLAMF1L, ICOS, ICOSL, GITR, GITRL, DAP10, DAP10L, DAP12, DAP12L, KIRDS2, CD2, LFA-1, Fyn, FynL, Lck, LckL, LAT, LATL, LRP, LRPL, BAFFR, SLAMF7, NKp80, NKp44, NKp30, NKp46, NOTCH1, NOTCH1L, NOTCH2, NOTCH2L, NOTCH3, NOTCH3L, NOTCH4, NOTCH4L, ROR2, ROR2L, Ryk, RykL, Slp76, Slp76L, pTα, pTαL, TCRα, TCRβ, HAVCR1 (TIM1), TIM1L, TRIM, TRIML, Zap70, Zap70L, PTCH2, PTCH2L, CD122, CD132, CD226, CD160, CD19, CARD11, CARD11L, CRTAM (CD355), IL2Rβ, IL2Rγ, IL7Ra, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1, SLAMF4, CD84, CD96, CEACAM1, CRT AM, Ly9 (SLAMF3), CD160, PSGL1, CD100, SLAMF6 (NTB-A, Lyl08), SLAM, BLAME (SLAMF8), SELPLG, LTBR, PAG/Cbp, NKG2D, NKG2DL, or NKG2C, FcRα, FcRαL, FcRβ, FcRβL, FcRγ, FcRγL, or CD47.

31. The protein of claim 27 having the sequence as set forth in SEQ ID NO: 143, SEQ ID NO: 144, SEQ ID NO: 145, SEQ ID NO: 147, SEQ ID NO: 149, or SEQ ID NO: 150.

32. The protein of claim 27, encoded by the sequence as set forth in SEQ ID NO: 146 or SEQ ID NO: 148.

33. A protein comprising an inverted intracellular signaling domain of a Type I protein and an extracellular domain of a Type II protein, wherein the inverted intracellular signaling domain is N-terminal to the extracellular domain and wherein the inverted intracellular signaling domain and the extracellular domain are linked through a transmembrane domain.

34. The protein of claim 33, wherein the extracellular domain is not inverted.

35. The protein of claim 33, wherein the Type I protein is a co-stimulatory protein.

36. The protein of claim 33, wherein the Type II protein is a co-stimulatory protein ligand.

37. The protein of claim 35, wherein the co-stimulatory protein is 4-1BB, OX40, CD40, CD30, CD30L, CD27, DR3, HVEM, SLAMF1, ICOS, GITR, CD25, CD28, CD79A, CD79B, CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TIM1, TRIM, Zap70, or PTCH2.

38. The protein of claim 35, wherein the co-stimulatory protein is 4-1BB, OX40, CD40, CD30, CD27, DR3, or HVEM.

39. The protein of claim 36, wherein the co-stimulatory protein ligand is 4-1BBL, OX40L, CD40L, CD30L, CD27L, DR3L, LIGHT, SLAMF1L, ICOSL, GITRL, CD80, CD86, CD79AL, CD79BL, CARD11 L, DAP10L, DAP12L, FcRαL, FcRβL, FcRγL, FLT3L, FynL, LckL, LATL, LRPL, NKG2DL, NOTCH1L, NOTCH2L, NOTCH3L, NOTCH4L, ROR2L, RykL, Slp76L, pTαL, TIM1L, TRIML, Zap70L, or PTCH2L

40. The protein of claim 36, wherein the co-stimulatory protein ligand is 4-1BBL, OX40L, CD40L, CD30L, CD27L, DR3L, FLT3L, or LIGHT.

41. The protein of claim 2, 27, or 33 further comprising a signal peptide.

42. A nucleotide comprising a gene encoding a protein of claim 2, 27, or 33.

43. The nucleotide of claim 42, wherein the nucleotide comprises regulatory elements comprising an inducible promoter.

44. A cell genetically modified to express a protein of claim 2, 27, or 33.

45. The cell of claim 44, wherein the cell is an immune cell.

46. The cell of claim 45, wherein the cell is a T cell or a natural killer (NK) cell.

47. The cell of claim 46, wherein the T cell is a CD4+ or a CD8+ T cell.

48. The cell of claim 45, wherein the cell is an induced pluripotent stem cell (iPSC), a tumor-infiltrating lymphocyte (TIL), a marrow-infiltrating lymphocyte (MIL), a natural killer T cell (NKT), a mucosal-associated invariant T (MATT) cell, a B cell, a dendritic cell, a monocyte or a macrophage.

49. A method of genetically-modifying an immune cell to express a protein of claim 2, 27, or 33 comprising introducing a nucleotide of claim 42 into the immune cell.

50. A formulation comprising an immune cell of claim 44 and a pharmaceutically acceptable carrier.

51. A nanoparticle comprising a nucleotide of claim 42.

52. The nanoparticle of claim 51, further comprising a cell targeting ligand.

53. The nanoparticle of claim 52, wherein the cell targeting ligand binds a T cell surface marker.

54. The nanoparticle of claim 53, wherein the T cell surface marker is CD3, CD4, or CD8.

55. The nanoparticle of claim 52, wherein the cell targeting ligand binds an iPSC marker, a TIL marker, a MIL marker, an NKT marker, a MATT cell marker, a B cell marker, a dendritic cell marker, a monocyte marker, or a macrophage marker.

56. A composition comprising a nanoparticle of claim 51 and a pharmaceutically acceptable carrier.

57. A method of modulating an immune response in a subject in need thereof comprising administering a therapeutically effective amount of a nucleotide of claim 42, a formulation of claim 50 or a nanoparticle of claim 51 to the subject, thereby modulating the immune response in the subject thereof.

58. The method of claim 57, wherein the modulating is stimulating.

59. The method of claim 57, wherein the modulating increases stimulatory intracellular signaling in immune cells within the subject.

60. The method of claim 59, wherein the increased stimulatory intracellular signaling in immune cells within the subject results in increased immune cell proliferation and/or increased immune cell activation in the subject, as compared to a comparable subject who was not administered the therapeutically effective amount.

61. The method of claim 57, wherein the modulating promotes an anti-cancer effect or an anti-infection effect in the subject.
